(19) United States
(12) Patent Application Publication
(10) Pub. No.: US 2023/0365488 A1
(43) Pub. Date: Nov. 16, 2023

(54) TRUXILLIC ACID MONOESTER-DERIVATIVES AS SELECTIVE FABP5 INHIBITORS AND PHARMACEUTICAL COMPOSITIONS AND USES THEREOF

(71) Applicant: The Research Foundation for The State University of New York, Albany, NY (US)

(72) Inventors: Iwao Ojima, Port Jefferson, NY (US); Martin Kaczocha, Dix Hills, NY (US); Monaf Awwa, Stony Brook, NY (US); Antonella Pepe, Stony Brook, NY (US)

(73) Assignee: The Research Foundation for The State University of New York, Albany, NY (US)

(21) Appl. No.: 18/248,232

(22) PCT Filed: Oct. 8, 2021
(86) PCT No.: PCT/US2021/054174

§ 371 Date: Apr. 6, 2023

Related U.S. Application Data
(60) Provisional application No. 63/089,422, filed on Oct. 8, 2020.

(51) Int. Cl. C07C 69/757; C07C 255/55; C07D 317/54; C07D 209/34; C07D 319/18; C07D 215/227; C07D 239/26; A61P 35/04; A61K 31/337; A61K 31/277; A61K 31/36; A61K 31/216; A61K 31/404; A61K 31/357; A61K 31/4704; A61K 31/505

(52) U.S. Cl. CPC C07C 69/757; C07C 255/55; C07D 317/54; C07D 209/34; C07D 319/18; C07D 215/227; C07D 239/26; A61P 35/04; A61K 31/337; A61K 31/277; A61K 31/36; A61K 31/216; A61K 31/404; A61K 31/357; A61K 31/4704; A61K 31/505; C07C 2601/04; C07C 2603/18; C07C 2602/08

(57) ABSTRACT

The present invention provides a compound, and method of selectively inhibiting the activity of a Fatty Acid Binding Protein (FABP) comprising contacting the FABP with a compound, said compound having the structure:

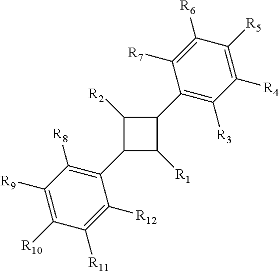

This application claims priority of U.S. Provisional Application No. 63/089,422, filed Oct. 8, 2020, the contents of which are hereby incorporated by reference.

This invention was made with government support under DA035923 and CA237154 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Fatty Acid Binding Proteins (FABPs)

Lipids require a variety of fatty acid binding protein (FABP) chaperones or transporters to carry them throughout cells due to their insolubility in water (Furuhashi, M. & Hotamisligil, G. S. 2008; Kaczocha, M. et al. 2009). As Table 1 shows, there are 10 human FABPs with considerable tissue specificity (Smathers, R. L. & Petersen, D. R. 2011). For instance, FABP3 (heart FABP), FABP5 (epidermal FABP), FABP7 (brain FABP) and FABP8 (myelin FABP) are all expressed in nervous and other tissues, while FABP1 (liver FABP) and FABP4 (adipose FABP) are abundantly expressed in the liver and adipose tissue (Veerkamp, J. H. & Zimmerman, A. W. 2001).

TABLE 1
Human fatty acid binding proteins (FABPs) and their localizations

| FABPs | Localization |
|---|---|
| FABP1 (Liver) | Liver, intestine, pancreas, kidney, lung, stomach |
| FABP2 (Intestinal) | Intestine, liver |
| FABP3 (Heart) | Cardiac and skeletal muscle, brain, kidney, lung, |
| | stomach, testis, adrenal gland, mammary gland, |
| | placenta, ovary, brown adipose tissue |
| FABP4 (Adipocyte) | Adipocytes, macrophages, dendritic cells, skeletal |
| | muscle fibres |
| FABP5 (Epidermal) | Skin, tongue, adipocyte, macrophage, dendritic cells, |
| | mammary gland, brain, stomach, intestine, kidney, |
| | liver, lung, heart, skeletal muscle, testis, retina, lens, |
| | spleen, placenta |
| FABP6 (Ileal) | Ileum, ovary, adrenal gland, stomach |
| FABP7 (Brain) | Brain, central nervous system (CNS), glial cell, |
| | retina, mammary gland |
| FABP8 (Myelin) | Peripheral nervous system, Schwann cells |
| FABP9 (Testis) | Testis, salivary gland, mammary gland |
| FABP12 | Retinoblastoma cell |

FABP5 and FABP7 Inhibitors as the Next-Generation Therapeutics for Chronic Pain Control

Recently, it has been shown that FABPs play a critical role in the inactivation pathway for anandamide (an endocannabinoid) by fatty acid amide hydrolase (FAAH), an enzyme localized on the endoplasmic reticulum (FIG. 1) (Kaczocha, M. et al. 2009; Berger, W. T. et al. 2012; Deutsch, D. G. 2016). The inhibition of FAAH and FABPs decreases the hydrolysis of anandamide and its uptake into cells, respectively, thereby raising levels of extracellular anandamide, which targets cannabinoid (CB) receptors (Howlett, A. C. et al. 2011; Kaczocha, M., et al. 2012; Ahn, K., et al. 2009). Consequently, the elevated levels of endocannabinoids result in beneficial pharmacological effects on stress, pain and inflammation, and also ameliorate the effects of drug withdrawal. FAAH inhibitors had been extensively studied as a potential therapy for anxiety disorder, Parkinson's disease, chronic pain of multiple sclerosis, cancer, hypertension, and obesity until one of the lead clinical candidates was found to be fatal in the Phase I human clinical trials (Mallet, C. Et al. 2016). In contrast to FAAH, which is distributed throughout the body, human FABPs have considerable tissue specificity as shown in Table 1. FABPs, in particular FABP5 and FABP7, have been identified as intracellular transporters for the endocannabinoid, “anandamide” (N-arachidonoylethanol-amine: AEA) (Kaczocha, M. et al. 2009).

FABP Inhibitors as the Next-Generation Cancer Chemotherapeutics for Drug-Resistant Prostate Cancer

It has been shown that fatty acid synthase (FASN) and monoacylglycerol lipase (MAGL) activities promote tumorigenesis in multiple cancer types including cancers of the prostate, skin, and breast (Regula, N. et al. 2016; Rossi, S. et al 2003; Nomura, D. K. et al. 2010; Nomura, D. K. et al. 2011; Ahmad, I. et al. 2016; Baba, Y., et al. 2017; Alwarawrah, Y., 2016). FASN is an enzyme that synthesizes de novo fatty acids and MAGL is an enzyme that cleaves 2-monoacylglycerols to generate free fatty acids (FIG. 2). Fatty acids are essential for the biosynthesis of membrane lipids, as well as energy use via S-oxidation, but fatty acids and their metabolites also function as agonists of the nuclear receptor peroxisome proliferator-activated receptor gamma (PPARγ). PPARγ regulates the expression of proangiogenic genes, which are overexpressed in metastatic prostate cancers and associated with shorter patient survival (Ahmad, I. et al. 2016; Forootan, F. S. et al. 2014; Bao, Z. et al. 2013). Furthermore, fatty acid-derived ligands activate PPAR3/6, which also promotes cancer cell survival and tumor growth (Her, N. G. et al. 2013; Schug, T. T. et al. 2007; Levi, L. et al. 2015). Thus, fatty acid signaling is linked to cancer aggression and metastasis.

Fatty acid-binding protein 5 (FABP5) is a member of a class of intracellular lipid chaperones that transports fatty acids to PPARγ, which leads to increased expression of proangiogenic factors, including vascular endothelial growth factor (VEGF), resulting in a metastatic phenotype (Nomura, D. K. et al. 2011; Baba, Y., et al. 2017; Alwarawrah, Y., 2016; Forootan, F. S. et al. 2014; Bao, Z. et al. 2013; Her, N. G. et al. 2013). Although a normal prostate lacks FABP5 expression, it becomes highly expressed in prostate cancer, and higher levels of its expression are linked to increased Gleason scores (Fujita, K. et al. 2017), hence advanced metastatic prostate tumors express the highest levels of FABP5 (Nomura, D. K. et al. 2011; Baba, Y., et al. 2017; Her, N. G. et al. 2013; Schug, T. T. et al. 2007). In correlation to this expression pattern, prostate cancer cell lines with low metastatic potential do not display FABP5 expression, while prostate cancer cell lines with high metastatic potential exhibit elevated levels of FABP5 expression (Nomura, D. K. et al. 2011; Levi, L. et al. 2015). Moreover, the introduction of FABP5 to prostate cancer cell lines with low metastatic potential enhances cell migration, invasion, and tumor formation, whereas FABP5 inhibition in prostate cancer cell lines with high metastatic potential suppresses metastasis (Ahmad, I. et al. 2016; Alwarawrah, Y., 2016; Levi, L. et al. 2015).

### SUMMARY OF THE INVENTION

The present invention provides a compound having the structure:

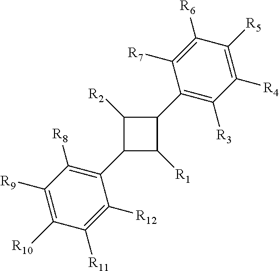

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

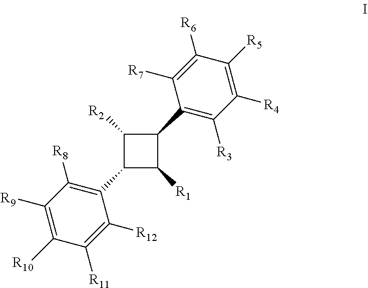

then
one of R₁ or R₂ is —C(═O)OH and the other of R₁ or R₂ is —C(═O)R₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)R₁₃ where R₁₃ is methyl, 2-propyl, pentyl, octyl, —CH₂C(O)CH₃, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl, or —C(═O)O-alkyl-R₁₄ where the alkyl is a branched C₂ alkyl and the R₁₄ is phenyl or the alkyl is a C₁ alkyl and the R₁₄ is phenyl, 4-methoxyphenyl, 4-fluorophenyl, 4-bromophenyl, or 9-fluorene,
wherein when one of R₁ or R₂ is —C(═O) OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene, 2-naphthalene, 2-phenylcyclohexyl, or —C(═O)O-alkyl-R₁₄ where the alkyl is a C₁ alkyl and the R₁₄ is 9-fluorene,
wherein when one of R₁ or R₂ is —C(═O) OH and R₄, R₅, R₆, R₇, R₉, R₁₀, R₁₁ and R₁₂ are each H and R₃ and R₈ are each —Cl or —Br, then the other of R₁ or R₂ is other than —C(═O)OR₁₃ where R₁₃ is 2-phenylcyclohexyl,
wherein when one of R₁ or R₂ is —C(═O) OH and R₄, R₅, R₆, R₉, R₁₀, and Ru are each H and R₃, R₇, R₈ and R₁₂ are each —Cl, then the other of R₁ or R₂ is other than —C(═O) OR₁₃ where R₁₃ is 2-phenylcyclohexyl,
wherein when one of R₁ or R₂ is —C(═O) OH and R₃, R₄, R₆, R₇, R₈, R₉, R₁₁, and R₁₂ are each H and R₅ and R₁₀ are each —OH, then the other of R₁ or R₂ is other than —C(═O) OR₁₃ where R₁₃ is 1-naphthalene,
wherein when one of R₁ or R₂ is —C(═O) OH and R₃, R₆, R₇, R₈, R₁₁, and R₁₂ are each H, R₄ and R₉ are each OCH₃, and R₅ and R₁₀ are each —OH, then the other of R₁ or R₂ is other than —C(═O) OR₁₃ where R₁₃ is 1-naphthalene,
wherein when the compound has the stereochemistry of structure II

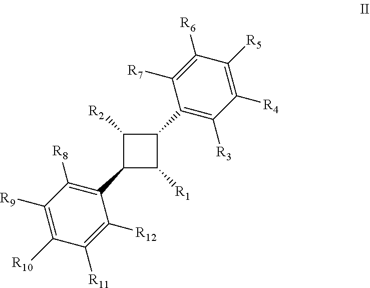

then
one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene or 2-methylphenyl, or —C(═O)O-alkyl-R₁₄ where the alkyl is a branched C₂ alkyl and the R₁₄ is phenyl, wherein when the compound has the stereochemistry of structure III

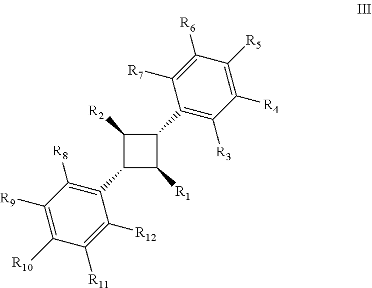

then
one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

The present invention also provides a compound having the structure:

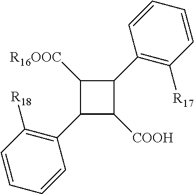

wherein
   R₁₆ is cycloalkyl, alkylcycloalkyl, aryl or alkylaryl, and R₁₇ and R₁₈ are each independently, H or —OCH₃,
wherein when the compound has the stereochemistry of structure IV

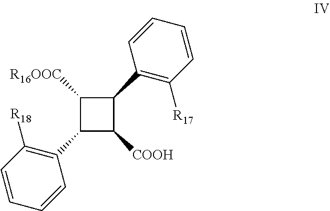

then
R₁₆ is cycloalkyl, alkylcycloalkyl, aryl or alkylaryl, and R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, benzyl, methylbenzyl, 4-methoxybenzyl, 4-fluorobenzyl, 4-bromobenzyl, —CH₂-9-fluorene, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl,
wherein when R₁₇ and R₁₈ are each —OCH₃, then R₁₆ is other than 1-naphthalene, 2-naphthalene, 2-phenylcyclohexyl, or —CH₂-9-fluorene,
wherein when the compound has the stereochemistry of structure V

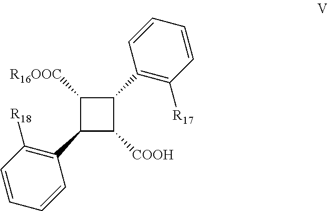

then
   R₁₆ is cycloalkyl, alkylcycloalkyl, aryl or alkylaryl, and
   R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, methylbenzyl, 1-naphthalene, 2-naphthalene or 2-methylphenyl,
wherein when the compound has the stereochemistry of structure VI

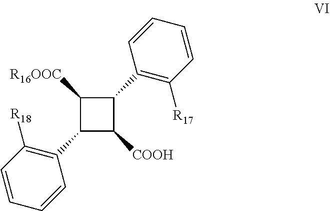

then
   R₁₆ is cycloalkyl, alkylcycloalkyl, aryl or alkylaryl, and
   R₁₇ and R₁₈ are each H or —OCH₃,
or an enantiomer or racemate thereof;
or a pharmaceutically acceptable salt thereof.

The present invention also provides a method of treating cancer in a subject comprising administering to the subject an effective amount of a compound having the structure:

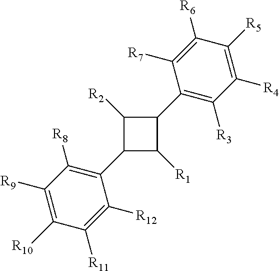

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

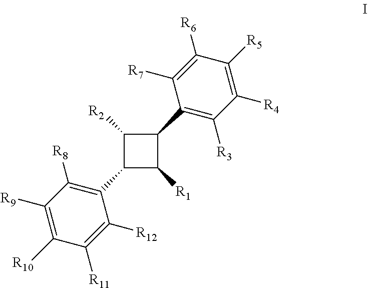

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure II

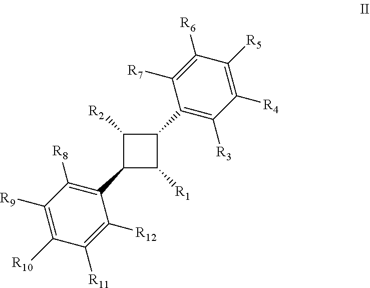

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

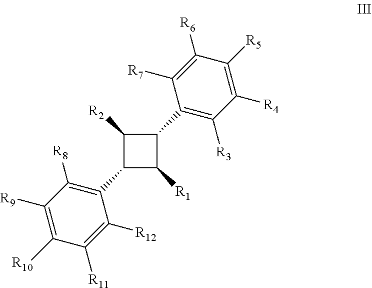

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

The present invention also provides a method of treating cancer in a subject comprising administering to the subject an effective amount of a compound having the structure:

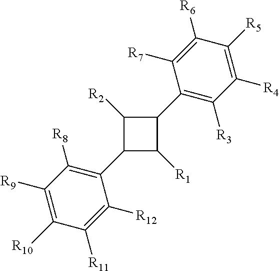

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

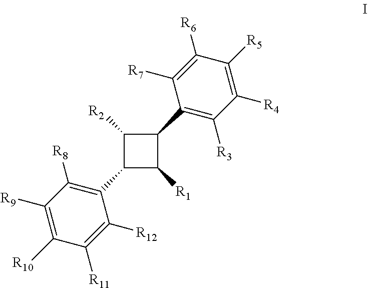

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O) OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene or —C(═O)O-alkyl-R₁₄ where the alkyl is a C₁ alkyl and the R₁₄ is 9-fluorene,
wherein when the compound has the stereochemistry of structure II

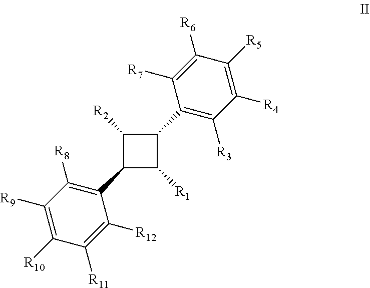

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

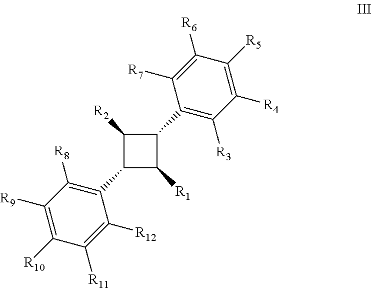

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

The present invention also provides a method of treating pain in a subject without the side-effects of excessive inhibition of FABP3 comprising administering to the subject an effective amount of a compound having the structure:

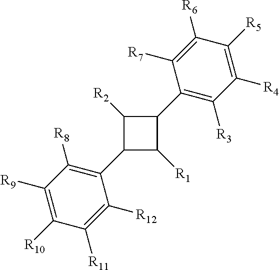

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

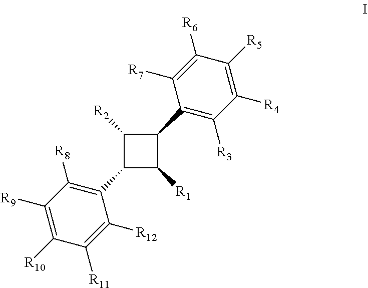

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure II

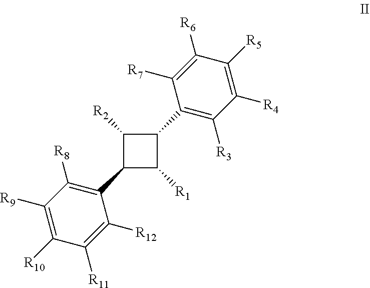

then
one of R₁ or R₂ is —C(═O) OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

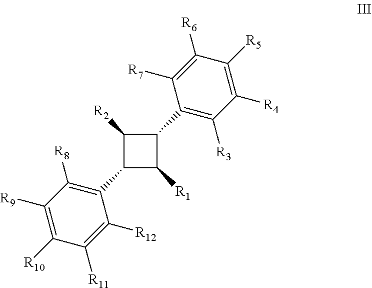

one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

### BRIEF DESCRIPTION OF THE FIGURES

FIG. 1: Scheme demonstrating the key roles of FABP and FAAH in the inactivation of anandamide inactivation and FABP drug target. Anandamide crosses the membrane by diffusion but requires FABPs for transport through the cytoplasm to the endoplasmic reticulum for breakdown by FAAH. FABP inhibitors prevent AEA from being delivered to FAAH for breakdown resulting in increased AEA levels at the receptor.

FIG. 2: Scheme demonstrating how FABP5 inhibitors may serve as the next-generation chemotherapy agents.

FIGS. 3A-3B: Cytotoxicity of 1y (SBFI-102) (FIG. 3A) or 1w (SBFI-103) (FIG. 3B) in PC3, DU-145, 22Rv1, RWPE-1, and WI-38 cells (n≥3).

FIGS. 4A-4F: Cytotoxicity of PC3, DU-145, and 22Rv1 cells following combinatorial treatment with docetaxel and 1y (SBFI-102) or 1w (SBFI-103). Cytotoxicity of PC3 cells incubated with docetaxel in the presence of A) 1y (SBFI-102) or B) 1w (SBFI-103) (n≥3). Cytotoxicity of DU-145 cells incubated with docetaxel in the presence of C) 1y (SBFI-102) or D) 1w (SBFI-103) (n≥3). Cytotoxicity of 22Rv1 cells incubated with docetaxel in the presence of E) 1y (SBFI-102) or F) 1w (SBFI-103) (n≥3).

FIG. 5A-5F: Cytotoxicity of PC3, DU-145, and 22Rv1 cells following combinatorial treatment with cabazitaxel and 1y (SBFI-102) or 1w (SBFI-103). Cytotoxicity of PC3 cells incubated with cabazitaxel in the presence of A) 1y (SBFI-102) or B) 1w (SBFI-103) (n≥3). Cytotoxicity of DU-145 cells incubated with cabazitaxel in the presence of C) 1y (SBFI-102) or D) 1w (SBFI-103) (n≥3). Cytotoxicity of 22Rv1 cells incubated with cabazitaxel in the presence of E) 1y (SBFI-102) or F) 1w (SBFI-103) (n≥3).

FIG. 6A-6D: Inhibition of subcutaneous tumor growth by docetaxel or FABP5 inhibitors. PC3 cells (1×10⁶) were implanted subcutaneously into male BALB/c nude mice. From day 15 onwards, mice were treated with vehicle, SBFI-102 (20 mg/kg, daily), SBFI-103 (20 mg/kg, daily), or docetaxel (5 mg/kg or 10 mg/kg, weekly). A) Tumor growth over the time course of treatments. B-D) Tumor volumes at days 25, 30, and 35, respectively. *P<0.05 versus vehicle treatment; **P<0.01 versus vehicle treatment; ***P<0.001 versus vehicle treatment; #P<0.05 versus 10 mg/kg docetaxel treatment; ##P<0.01 versus 10 mg/kg docetaxel treatment; (n=5).

FIG. 7A-7D: Inhibition of subcutaneous tumor growth by docetaxel and FABP5 inhibitors. PC3 cells (1×10⁶) were implanted subcutaneously into male BALB/c nude mice. From day 15 onwards, mice were treated with vehicle, SBFI-102 (20 mg/kg, daily) in combination with docetaxel (5 mg/kg, weekly), SBFI-103 (20 mg/kg, daily) in combination with docetaxel (5 mg/kg, weekly), or docetaxel (5 mg/kg or 10 mg/kg, weekly). A) Tumor growth over the time course of treatments. B-D) Tumor volumes at days 25, 30, and 35, respectively. **P<0.01 versus vehicle treatment; ***P<0.001 versus vehicle treatment; #P<0.05 versus 10 mg/kg docetaxel treatment; NS versus 10 mg/kg docetaxel treatment; (n=5).

### DETAILED DESCRIPTION OF THE INVENTION

The present invention provides a compound having the structure:

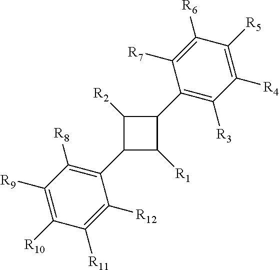

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

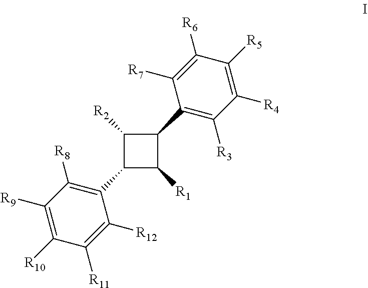

then
one of R₁ or R₂ is —C(═O)OH and the other of R₁ or R₂ is —C(═O)R₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl, or —C(═O)O-alkyl-R₁₄ where the alkyl is a branched C₂ alkyl an_{d} the R₁₄ is phenyl or the alkyl is a C₁ alkyl and the R₁₄ is phenyl, 4-methoxyphenyl, 4-fluorophenyl, 4-bromophenyl, or 9-fluorene,
wherein when one of R₁ or R₂ is —C(═O) OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene, 2-naphthalene, 2-phenylcyclohexyl, or —C(═O)O-alkyl-R₁₄ where the alkyl is a C₁ alkyl and the R₁₄ is 9-fluorene,
wherein when one of R₁ or R₂ is —C(═O) OH and R₄, R₅, R₆, R₇, R₉, R₁₀, R₁₁ and R₁₂ are each H and R₃ and R₈ are each —Cl or —Br, then the other of R₁ or R₂ is other than —C(═O)OR₁₃ where R₁₃ is 2-phenylcyclohexyl,
wherein when one of R₁ or R₂ is —C(═O) OH and R₄, R₅, R₆, R₉, R₁₀, and Ru are each H and R₃, R₇, R₈ and R₁₂ are each —Cl, then the other of R₁ or R₂ is other than —C(═O) OR₁₃ where R₁₃ is 2-phenylcyclohexyl,
wherein when one of R₁ or R₂ is —C(═O) OH and R₃, R₄, R₆, R₇, R₈, R₉, R₁₁, and R₁₂ are each H and R₅ and R₁₀ are each —OH, then the other of R₁ or R₂ is other than —C(═O) OR₁₃ where R₁₃ is 1-naphthalene,
wherein when one of R₁ or R₂ is —C(═O) OH and R₃, R₆, R₇, R₉, R₁₁, and R₁₂ are each H, R₄ and R₉ are each OCH₃, and R₅ and R₁₀ are each —OH, then the other of R₁ or R₂ is other than —C(═O) OR₁₃ where R₁₃ is 1-naphthalene,
wherein when the compound has the stereochemistry of structure II

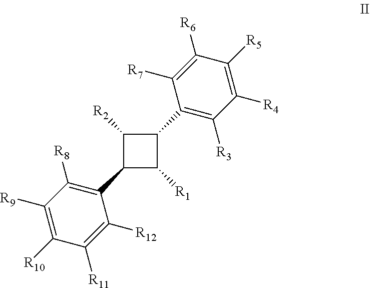

one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene or 2-methylphenyl, or —C(═O)O-alkyl-R₁₄ where the alkyl is a branched C₂ alkyl and the R₁₄ is phenyl,
wherein when the compound has the stereochemistry of structure III

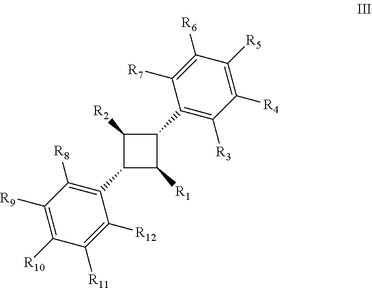

then
one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

In some embodiments of the above compound,
wherein
one of R₁ or R₂ is —C(═O)OR₁₃,
   wherein R₁₃ is cycloalkyl, aryl or heteroaryl; and
the other of R₁ or R₂ is —C(═O)OH.

In some embodiments of the above compound,
wherein
one of R₁ or R₂ is —C(═O)O-alkyl-R₁₄,
   wherein R₁₄ is cycloalkyl, aryl or heteroaryl; and
the other of R₁ or R₂ is —C(═O)OH.

In some embodiments of the above compound,
wherein
one of R₁ or R₂ is —C(═O)O—(C₁₋₆ alkyl)-R₁₄,
   wherein R₁₄ is cycloalkyl, aryl or heteroaryl; and the other of R₁ or R₂ is —C(═O)OH.

In some embodiments of the above compound,
wherein
one of R₁ or R₂ is —C(═O)O—CH₂—R₁₄,
   wherein R₁₄ is cycloalkyl, aryl or heteroaryl; and
the other of R₁ or R₂ is —C(═O)OH.

In some embodiments, the compound wherein R₁₃ or R₁₄ is a cycloalkyl that is substituted with a ring structure or fused to another ring structure.

In some embodiments, the compound wherein R₁₃ or R₁₄ is an aryl or heteroaryl that is substituted with a ring structure or fused to another ring structure.

In some embodiments, the compound wherein the aryl is substituted with an aryl, a substituted aryl, heteroaryl or substituted heteroaryl.

In some embodiments, the compound wherein the aryl is substituted with a halogen, —OH, CN, aryl, heteroaryl, or —O(alkyl).

In some embodiments, the compound wherein the aryl is substituted with a halogen, —OH, aryl, heteroaryl, or —O(alkyl).

In some embodiments, the compound wherein the aryl is substituted with an amide, aryl or hydroxyaryl.

In some embodiments, the compound wherein the aryl is substituted with a F, Cl, Br, —OH, triazolyl, C₂ alkynyl or —OCH₃.

In some embodiments, the compound wherein the aryl is substituted with a F, Cl, Br, —OH, I, —NHC(O)CH₃, triazolyl, C₂ alkynyl, phenyl, o-hydroxyphenyl or —OCH₃.

In some embodiments, the compound wherein the heteroaryl is substituted with an aryl, a substituted aryl, heteroaryl or substituted heteroaryl.

In some embodiments, the compound wherein the heteroaryl is substituted with a halogen, —OH, heteroaryl, C₂-C₆ alkynyl or —O(alkyl).

In some embodiments, the compound wherein the heteroaryl is substituted with an amide, aryl or hydroxyaryl.

In some embodiments, the compound wherein the heteroaryl is substituted with an F, Cl, Br, —OH, triazolyl, C₂ alkynyl or —OCH₃.

In some embodiments, the compound wherein the heteroaryl is substituted with a F, Cl, Br, —OH, I, —NHC(O)CH₃, triazolyl, C₂ alkynyl, phenyl, o-hydroxyphenyl or —OCH₃.

In some embodiments, the compound wherein the cycloalkyl is a substituted cycloalkyl.

In some embodiments, the compound wherein the cycloalkyl is a) substituted with a phenyl group, b) fused with a phenyl group, c) fused with a benzo group.

In some embodiments, the compound wherein the cycloalkyl is:

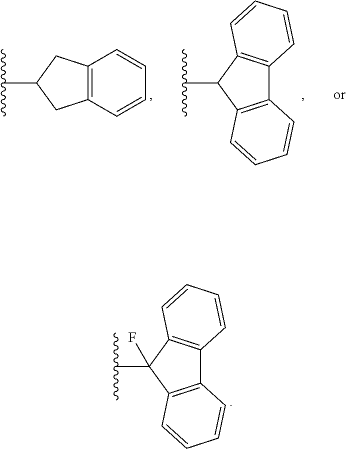

In some embodiments of the above compound,
wherein
one of R₁ or R₂ is

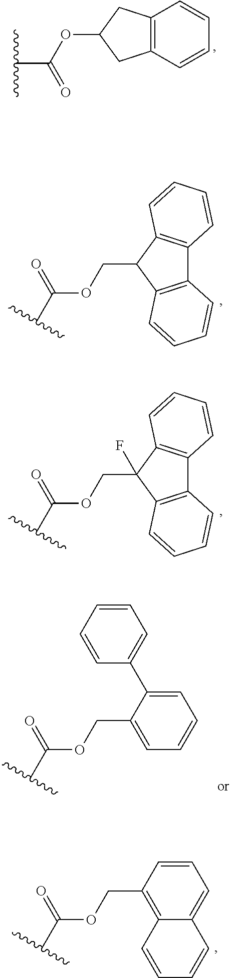

the other of R₁ or R₂ is —C(═O)OH.

In some embodiments of the above compound,
wherein
one of R₁ or R₂ is

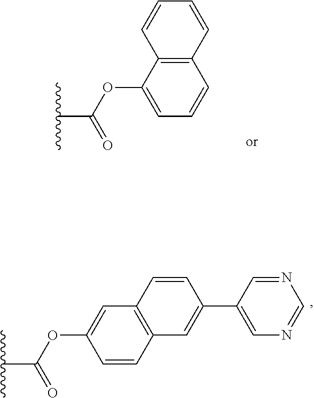 [SMILES: *C(=O)Oc1cccc2ccccc12 ; *C(=O)Oc1ccc2cc(-c3cncnc3)ccc2c1]

the other of R₁ or R₂ is —C(═O)OH.

In some embodiments of the above compound,
wherein
one of R₁ or R₂ is

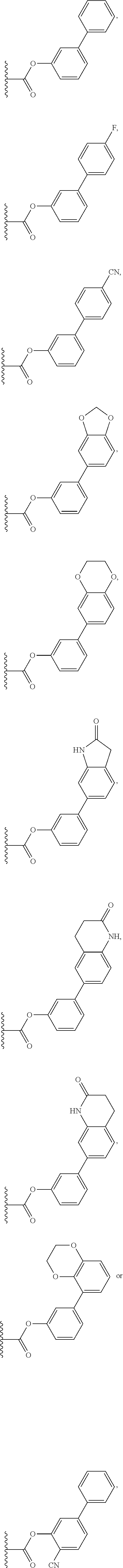

the other of R₁ or R₂ is —C(═O)OH.

In some embodiments of the above compound,
wherein
one of R₁ or R₂ is

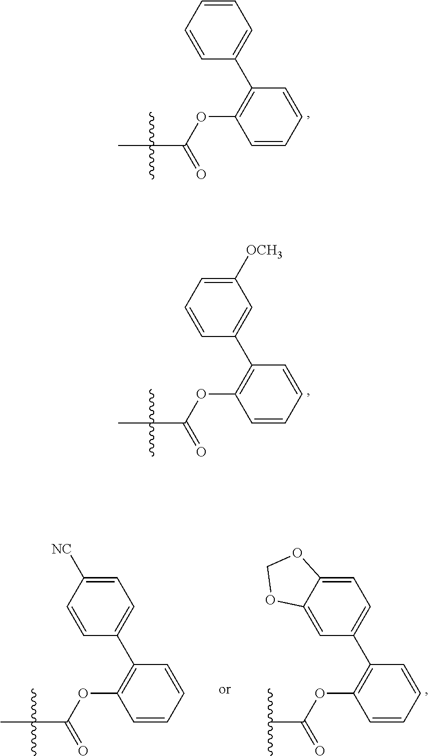 [SMILES: *C(=O)Oc1ccccc1-c1ccccc1 ; *C(=O)Oc1ccccc1-c1cccc(OC)c1 ; *C(=O)Oc1ccccc1-c1ccc(C#N)cc1 ; *C(=O)Oc1ccccc1-c1ccc2c(c1)OCO2]

the other of R₁ or R₂ is —C(═O)OH.

In some embodiments, the compound wherein R₃, R₄, R₅, R₆, R₇, R₈, R₉, R₁₀, R₁₁ and R₁₂ are each independently, —H, or —OR₁₅,
wherein R₁₅ is —H or C₁₋₁₀ alkyl.

In some embodiments, the compound wherein R₃, R₄, R₅, R₆, R₇, R₈, R₉, R₁₀, R₁₁ and R₁₂ are each independently, —H or —OCH₃.

In some embodiments, the compound wherein R₃, R₄, R₅, R₆, R₇, R₈, R₉, R₁₀, R₁₁ and R₁₂ are each —H.

In some embodiments, the compound wherein one of R₃, R₄, R₅, R₆, R₇, R₈, R₉, R₁₀, R₁₁ and R₁₂ is other than —H.

In some embodiments, the compound wherein two of R₃, R₄, R₅, R₆, R₇, R₈, R₉, R₁₀, R₁₁ and R₁₂ are other than —H.

In some embodiments, the compound wherein four of R₃, R₄, R₅, R₆, R₇, R₈, R₉, R₁₀, R₁₁ and R₁₂ are other than —H.

In some embodiments, the compound wherein R₄, R₅, R₆, R₇, R₉, R₁₀, R₁₁ and R₁₂ are each —H and R₃ and R₈ are each —OCH₃.

In some embodiments of the above compound,
wherein
one of R₁ or R₂ is —C(═O)OH and the other of R₁ or R₂ is —C(═O)OR₁₃ or —C(═O)O-alkyl-R₁₄,
   wherein
   R₁₃ is cycloalkyl or aryl, and
   R₁₄ is cycloalkyl or aryl; and
R₃, R₄, R₅, R₆, R₇, R₈, R₉, R₁₀, R₁₁ and R₁₂ are each independently, H or —OR₁₅,
wherein R₁₅ is H or C₁₋₁₀ alkyl.

In some embodiments of the above compound,
wherein
one of R₁ or R₂ is —C(═O)OH and the other of R₁ or R₂ is —C(═O)OR₁₃ or —C(═O)O-alkyl-R₁₄,
   wherein
   R₁₃ is cycloalkyl or aryl, and
   R₁₄ is cycloalkyl or aryl; and
R₃, R₄, R₅, R₆, R₇, R₈, R₉, R₁₀, R₁₁ and R₁₂ are each H.

In some embodiments of the above compound,
wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl or aryl, and
   R₁₄ is cycloalkyl or aryl; and R₄, R₅, R₆, R₇, R₉, R₁₀, R₁₁ and R₁₂ are each —H and R₃ and R₈ are each —OCH₃.

The present invention also provides a compound having the structure:

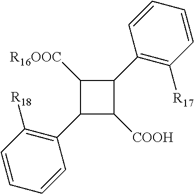

wherein
   R₁₆ is cycloalkyl, alkylcycloalkyl, aryl or alkylaryl, and R₁₇ and R₁₈ are each independently, H or —OCH₃,
wherein when the compound has the stereochemistry of structure IV

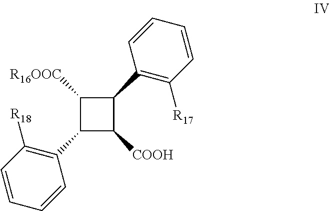

then
   R₁₆ is cycloalkyl, alkylcycloalkyl, aryl or alkylaryl, and
   R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, benzyl, methylbenzyl, 4-methoxybenzyl, 4-fluorobenzyl, 4-bromobenzyl, —CH₂-9-fluorene, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl,
wherein when R₁₇ and R₁₈ are each —OCH₃, then R₁₆ is other than 1-naphthalene, 2-naphthalene, 2-phenylcyclohexyl, or —CH₂-9-fluorene,
wherein when the compound has the stereochemistry of structure V

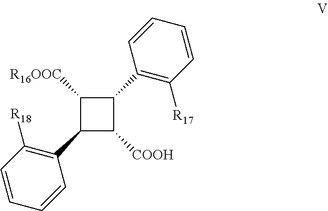

then
   R₁₆ is cycloalkyl, alkylcycloalkyl, aryl or alkylaryl, and
   R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, methylbenzyl, 1-naphthalene, 2-naphthalene or 2-methylphenyl,
wherein when the compound has the stereochemistry of structure VI

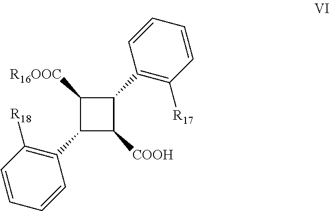

then
   R₁₆ is cycloalkyl, alkylcycloalkyl, aryl or alkylaryl, and
   R₁₇ and R₁₈ are each H or —OCH₃,
or an enantiomer or racemate thereof;
or a pharmaceutically acceptable salt thereof.

In some embodiments of the above compound,
wherein
R₁₆ is cycloalkyl, alkylcycloalkyl, aryl or alkylaryl.

In some embodiments of the above compound,
wherein
R₁₆ is

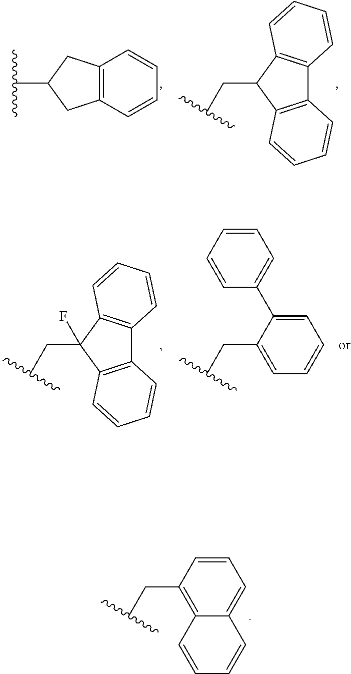 [SMILES: *C1Cc2ccccc2C1 ; *CC1c2ccccc2-c2ccccc21 ; *CC1(F)c2ccccc2-c2ccccc21 ; *Cc1ccccc1-c1ccccc1 ; *Cc1cccc2ccccc12]

In some embodiments of the above compound,
wherein
R₁₆ is

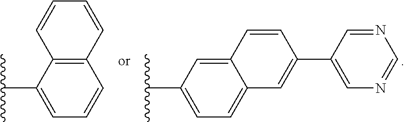

In some embodiments of the above compound,
wherein
R₁₆ is

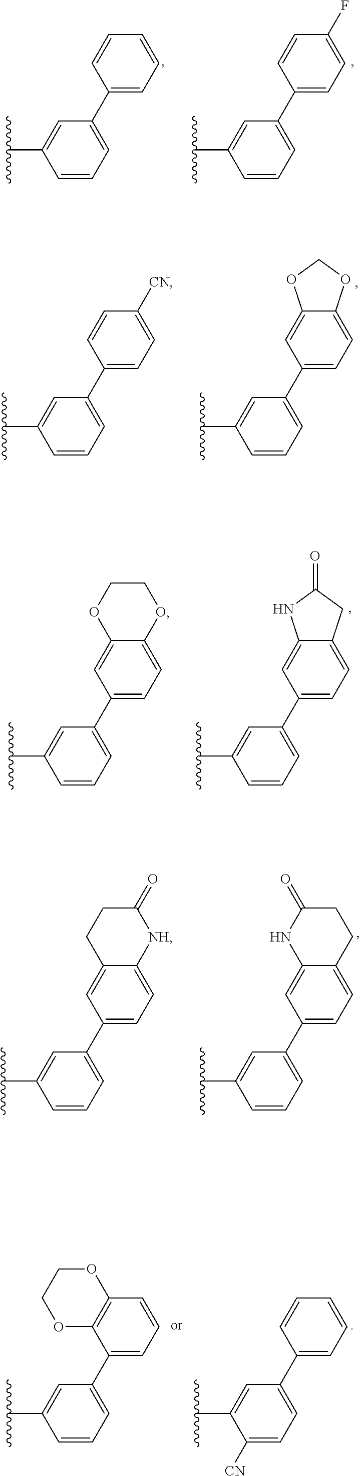

In some embodiments of the above compound,
wherein
R₁₆ is

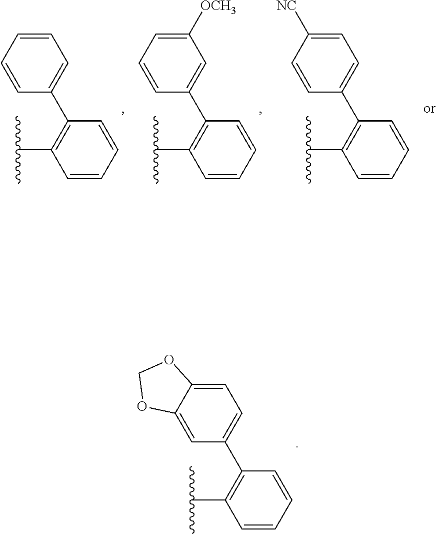

In some embodiments of the above compound,
wherein
R₁₇ and R₁₈ are each H.

In some embodiments of the above compound,
wherein
R₁₇ and R₁₈ are each —OCH₃.

In some embodiments, the compound having the structure:

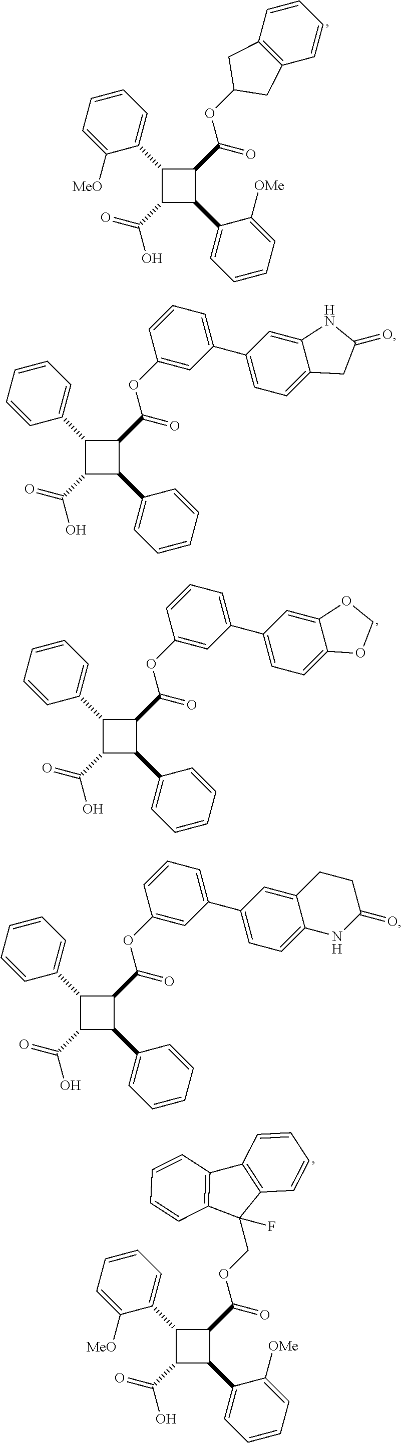

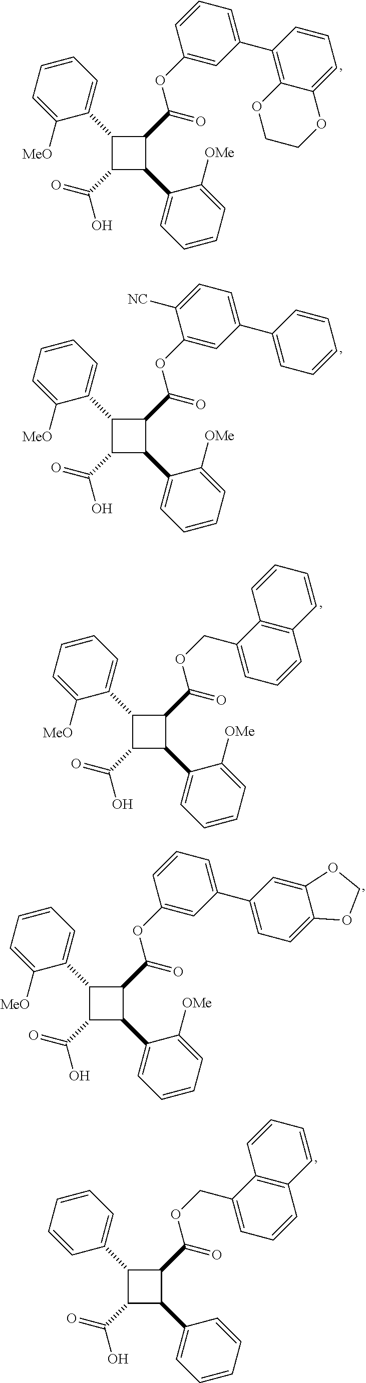

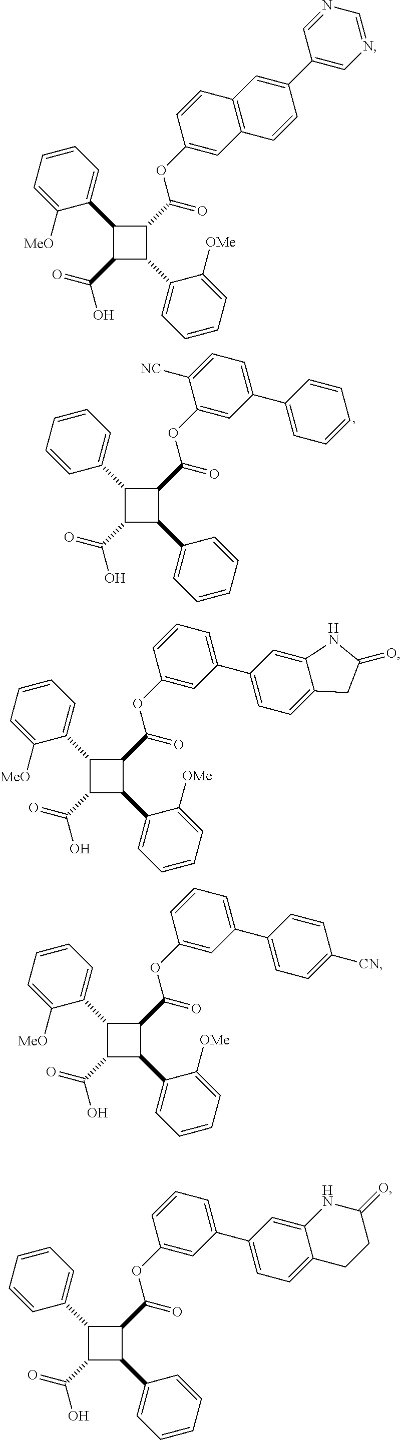

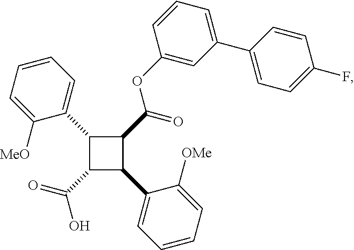 [SMILES: COc1ccccc1[C@H]1[C@H](C(=O)O)[C@H](c2ccccc2OC)[C@H]1C(=O)Oc1cccc(-c2ccc(F)cc2)c1]

or an enantiomer or racemate thereof;
or a pharmaceutically acceptable salt thereof.

In some embodiments, the compound having the structure:

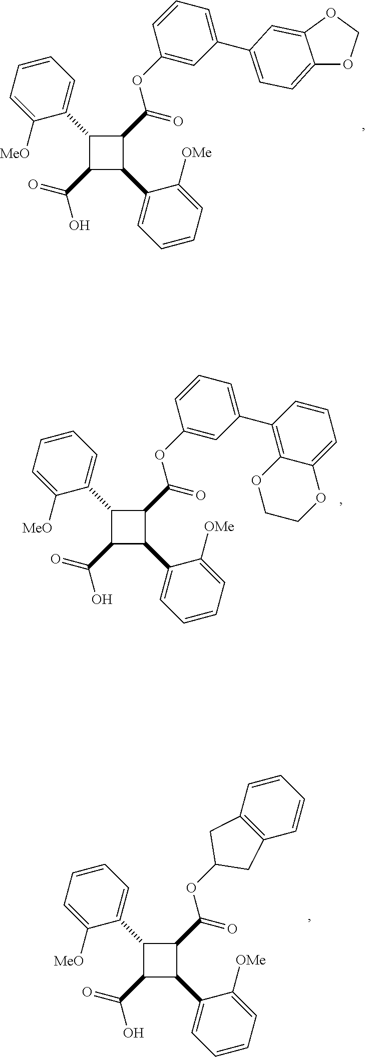 [SMILES: COc1ccccc1[C@H]1[C@H](C(=O)O)[C@H](c2ccccc2OC)[C@@H]1C(=O)Oc1cccc(-c2ccc3c(c2)OCO3)c1 ; COc1ccccc1[C@H]1[C@H](C(=O)O)[C@H](c2ccccc2OC)[C@@H]1C(=O)Oc1cccc(-c2cccc3c2OCCO3)c1 ; COc1ccccc1[C@H]1[C@H](C(=O)O)[C@H](c2ccccc2OC)[C@@H]1C(=O)OC1Cc2ccccc2C1]

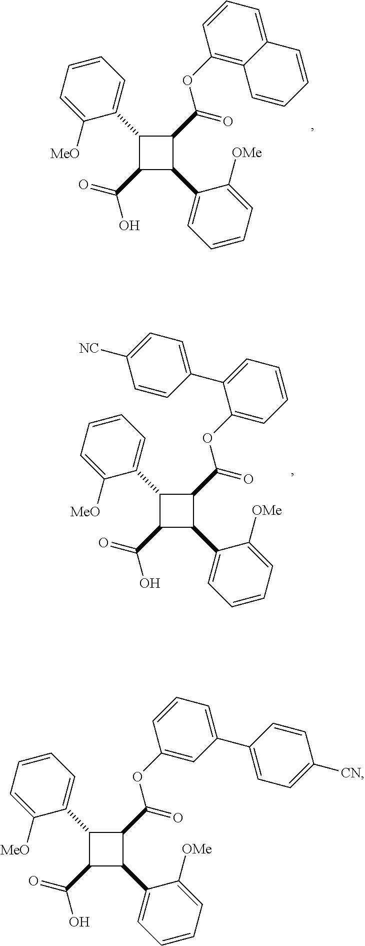 [SMILES: COc1ccccc1[C@H]1[C@H](C(=O)O)[C@H](c2ccccc2OC)[C@@H]1C(=O)Oc1cccc2ccccc12 ; COc1ccccc1[C@H]1[C@H](C(=O)O)[C@H](c2ccccc2OC)[C@@H]1C(=O)Oc1ccccc1-c1ccc(C#N)cc1 ; COc1ccccc1[C@H]1[C@H](C(=O)O)[C@H](c2ccccc2OC)[C@@H]1C(=O)Oc1cccc(-c2ccc(C#N)cc2)c1]

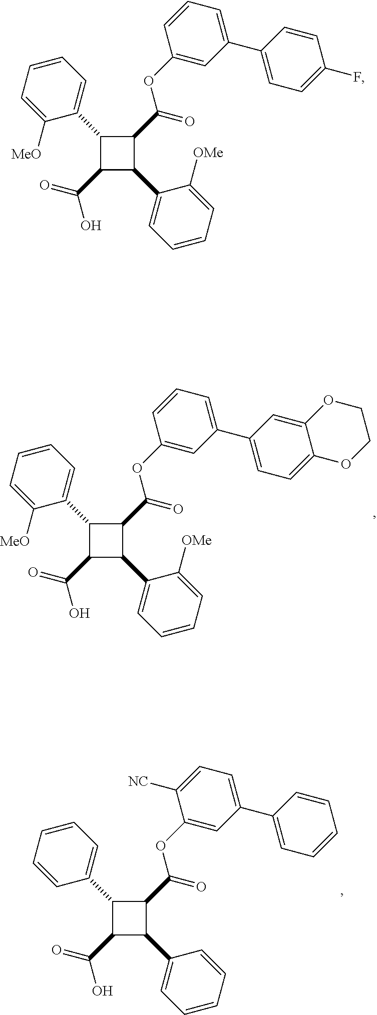 [SMILES: COc1ccccc1[C@H]1[C@H](C(=O)O)[C@H](c2ccccc2OC)[C@@H]1C(=O)Oc1cccc(-c2ccc(F)cc2)c1 ; COc1ccccc1[C@H]1[C@H](C(=O)O)[C@H](c2ccccc2OC)[C@@H]1C(=O)Oc1cccc(-c2ccc3c(c2)OCCO3)c1 ; N#Cc1ccc(-c2ccccc2)cc1OC(=O)[C@H]1[C@@H](c2ccccc2)[C@@H](C(=O)O)[C@@H]1c1ccccc1]

or an enantiomer or racemate thereof;
or a pharmaceutically acceptable salt thereof.

In some embodiments, the compound having the structure:

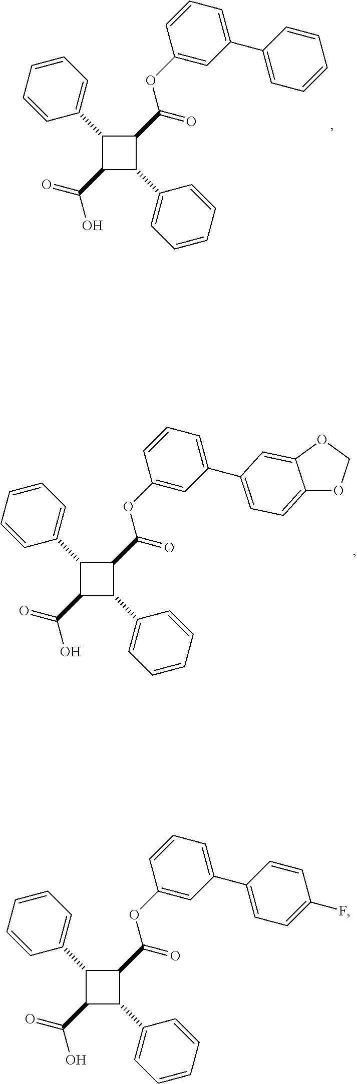 [SMILES: O=C(O)[C@@H]1[C@@H](c2ccccc2)[C@H](C(=O)Oc2cccc(-c3ccccc3)c2)[C@H]1c1ccccc1 ; O=C(O)[C@@H]1[C@@H](c2ccccc2)[C@H](C(=O)Oc2cccc(-c3ccc4c(c3)OCO4)c2)[C@H]1c1ccccc1 ; O=C(O)[C@@H]1[C@@H](c2ccccc2)[C@H](C(=O)Oc2cccc(-c3ccc(F)cc3)c2)[C@H]1c1ccccc1]

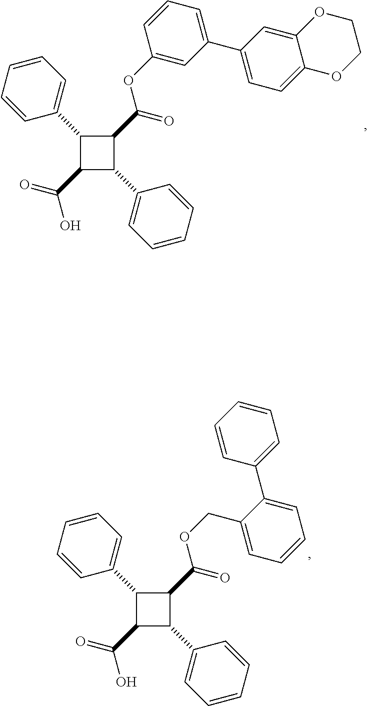 [SMILES: O=C(O)[C@@H]1[C@@H](c2ccccc2)[C@H](C(=O)Oc2cccc(-c3ccc4c(c3)OCCO4)c2)[C@H]1c1ccccc1 ; O=C(O)[C@@H]1[C@@H](c2ccccc2)[C@H](C(=O)OCc2ccccc2-c2ccccc2)[C@H]1c1ccccc1]

or an enantiomer or racemate thereof;
or a pharmaceutically acceptable salt thereof.

The present invention provides a pharmaceutical composition comprising the compound of the present invention and a pharmaceutically acceptable carrier.

The present invention provides a method of inhibiting binding of a Fatty Acid Binding Protein (FABP) to a FABP ligand in a cell comprising contacting the FABP with the compound of the present invention.

In some embodiments, wherein the FABP ligand is an endocannabinoid.

In some embodiments, wherein the FABP ligand is anandamide (AEA) or 2-arachidonoylglycerol (2-AG).

In some embodiments, wherein the FABP is FABP5 or FABP7.

The present invention provides a method of treating pain in a subject comprising administering to the subject the compound of the present invention.

In some embodiments, wherein the pain is nociceptive pain, neurogenic pain, inflammatory pain, or chronic pain.

In some embodiments, wherein the compound of the present invention is administered in an effective amount to inhibit binding of FABP to a FABP ligand in the subject.

In some embodiments, wherein the FABP is FABP5 or FABP7.

The present invention provides a method of treating cancer in a subject comprising administering to the subject an effective amount of a compound having the structure:

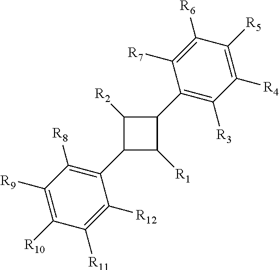

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

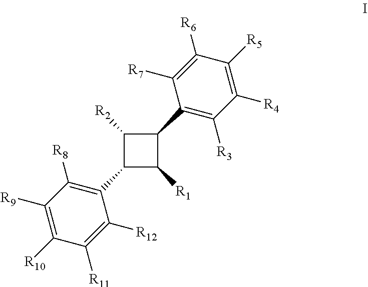

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure II

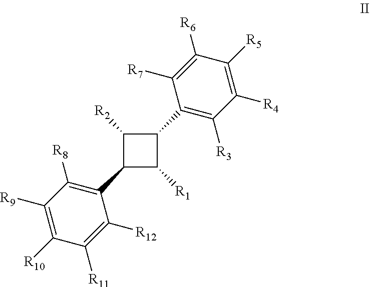

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

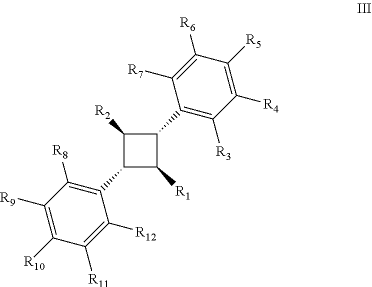

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

In the embodiment of the method, the compound can have the structure of any of the compound embodiments and any compound described herein.

The present invention provides a method of treating cancer in a subject comprising administering to the subject an effective amount of a compound having the structure:

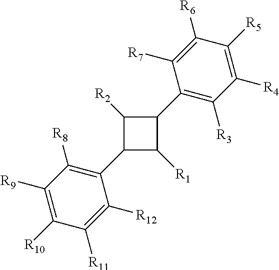

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

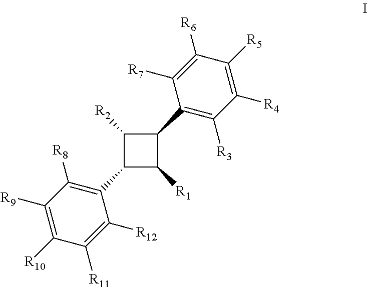

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O) OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene or —C(═O)O-alkyl-R₁₄ where the alkyl is a C₁ alkyl and the R₁₄ is 9-fluorene,
wherein when the compound has the stereochemistry of structure II

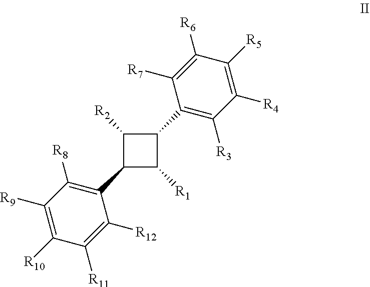

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

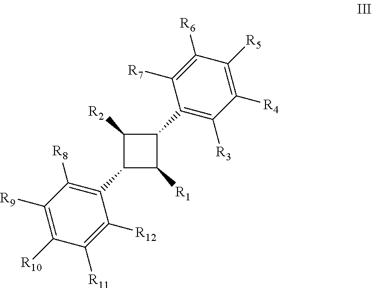

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

In the embodiment of the method, the compound can have the structure of any of the compound embodiments and any compound described herein.

In some embodiments, wherein the cancer is prostate cancer, skin cancer or breast cancer.

In some embodiments, wherein the cancer is drug-resistant prostate cancer.

In some embodiments, wherein the cancer is metastatic prostate cancer.

In some embodiments, wherein further comprising administering a taxane in combination with the compound of the present invention to the subject.

In some embodiments, wherein the taxane is docetaxel or cabazitaxel.

The present invention provides a method of treating pain in a subject without the side-effects of excessive inhibition of FABP3 comprising administering to the subject an effective amount of a compound having the structure:

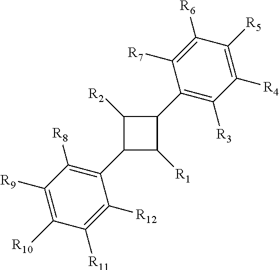

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

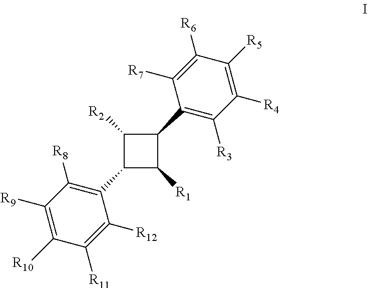

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure II

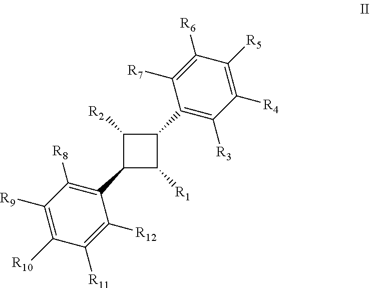

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III

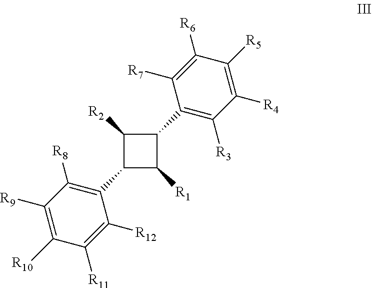

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

In the embodiment of the method, the compound can have the structure of any of the compound embodiments and any compound described herein.

In some embodiments, wherein the pain is nociceptive pain, neurogenic pain, inflammatory pain, or chronic pain.

In some embodiments of any of the above methods, comprising administering to the subject an effective amount of a compound having the structure:

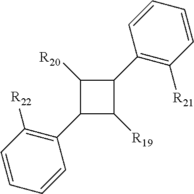

wherein
one of R₁₉ or R₂₀ is —C(═O)OH and the other of R₁₉ or R₂₀ is —C(═O)OR₂₃ or —C(═O)O-alkyl-R₂₄,
   wherein
   R₂₃ is cycloalkyl, aryl or heteroaryl, and
   R₂₄ is cycloalkyl, aryl or heteroaryl; and
R₂₁ and R₂₂ are each independently, H, —OH, —OR₂₅, or halogen
   wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure VII

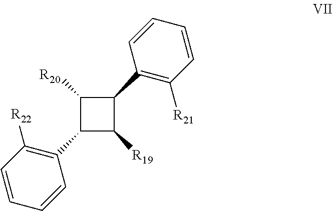

then
one of R₁₉ or R₂₀ is —C(═O)OH and the other of R₁₉ or R₂₀ is —C(═O)OR₂₃ or —C(═O)O-alkyl-R₂₄,
   wherein
   R₂₃ is cycloalkyl, aryl or heteroaryl, and
   R₂₄ is cycloalkyl, aryl or heteroaryl; and
R₂₁ and R₂₂ are each independently, H, —OH, —OR₂₅, or halogen
   wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure VIII

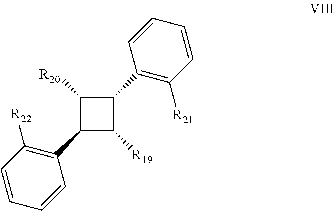

then
one of R₁₉ or R₂₀ is —C(═O)OH and the other of R₁₉ or R₂₀ is —C(═O)OR₂₃ or —C(═O)O-alkyl-R₂₄,
   wherein
   R₂₃ is cycloalkyl, aryl or heteroaryl, and
   R₂₄ is cycloalkyl, aryl or heteroaryl; and
R₂₁ and R₂₂ are each independently, H, —OH, —OR₂₅, or halogen
   wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure IX

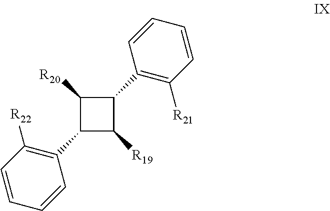

then
one of R₁₉ or R₂₀ is —C(═O)OH and the other of R₁₉ or R₂₀ is —C(═O)OR₂₃ or —C(═O)O-alkyl-R₂₄,
   wherein
   R₂₃ is cycloalkyl, aryl or heteroaryl, and
   R₂₄ is cycloalkyl, aryl or heteroaryl; and
R₂₁ and R₂₂ are each independently, H, —OH, —OR₂₅, or halogen
   wherein R₂₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

In some embodiments of any of the above methods, comprising administering to the subject an effective amount of a compound having the structure:

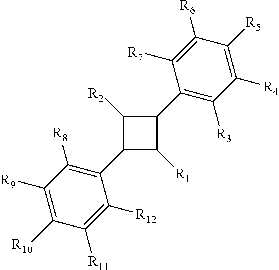

wherein
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I

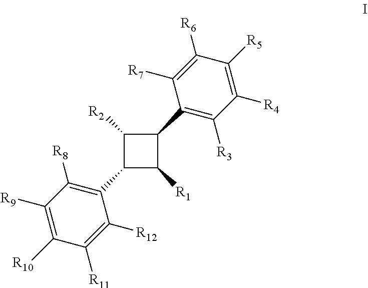

one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl, or —C(═O)O-alkyl-R₁₄ where the alkyl is a branched C₂ alkyl and the R₁₄ is phenyl or the alkyl is a C₁ alkyl and the R₁₄ is phenyl, 4-methoxyphenyl, 4-fluorophenyl, 4-bromophenyl, or 9-fluorene,
wherein when one of R₁ or R₂ is —C(═O)OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene, 2-naphthalene, 2-phenylcyclohexyl, or —C(═O)O-alkyl-R₁₄ where the alkyl is a C₁ alkyl and the R₁₄ is 9-fluorene,
wherein when one of R₁ or R₂ is —C(═O)OH and R₄, R₅, R₆, R₇, R₉, R₁₀, R₁₁ and R₁₂ are each H and R₃ and R₈ are each —Cl or —Br, then the other of R₁ or R₂ is other than —C(═O)OR₁₃ where R₁₃ is 2-phenylcyclohexyl,
wherein when one of R₁ or R₂ is —C(═O) OH and R₄, R₅, R₆, R₉, R₁₀, and Ru are each H and R₃, R₇, R₈ and R₁₂ are each —Cl, then the other of R₁ or R₂ is other than —C(═O) OR₁₃ where R₁₃ is 2-phenylcyclohexyl,
wherein when one of R₁ or R₂ is —C(═O) OH and R₃, R₄, R₆, R₇, R₈, R₉, R₁₁, and R₁₂ are each H and R₅ and R₁₀ are each —OH, then the other of R₁ or R₂ is other than —C(═O) OR₁₃ where R₁₃ is 1-naphthalene,
wherein when one of R₁ or R₂ is —C(═O) OH and R₃, R₆, R₇, R₈, R₁₁, and R₁₂ are each H, R₄ and R₉ are each OCH₃, and R₅ and R₁₀ are each —OH, then the other of R₁ or R₂ is other than —C(═O) OR₁₃ where R₁₃ is 1-naphthalene,
wherein when the compound has the stereochemistry of structure II

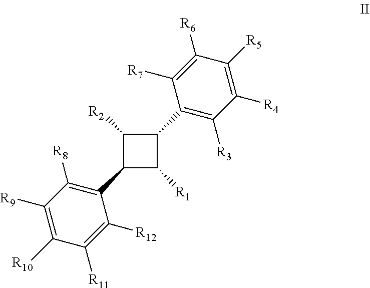

then
one of R₁ or R₂ is —C(═O)OH and the other of R₁ or R₂ is —C(═O)OR₁₃ or —C(═O) O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O) OR₁₃ where R₁₃ is methyl, 2-propyl, pentyl, octyl, —CH₂C(O)CH₃, 1-naphthalene, 2-naphthalene or 2-methylphenyl, or —C(═O)O-alkyl-R₁₄ where the alkyl is a branched C₂ alkyl and the R₁₄ is phenyl,
wherein when the compound has the stereochemistry of structure III

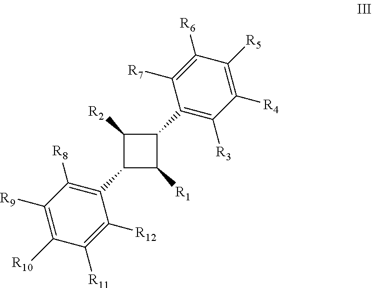

then
one of R₁ or R₂ is —C(═O)OH and the other of R₁ or R₂ is —C(═O)OR₁₃ or —C(═O)O-alkyl-R₁₄,
   wherein
   R₁₃ is cycloalkyl, aryl or heteroaryl, and
   R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
   wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

Compounds of the present invention include the following:

| Compound | Compound Structure |
|---|---|
| 1a | 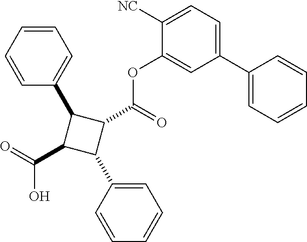 |
| 1b | 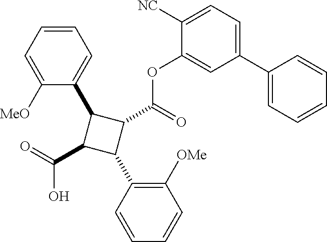 |
| 1c | 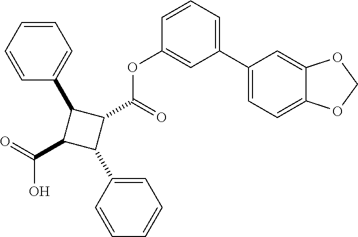 |
| 1d | 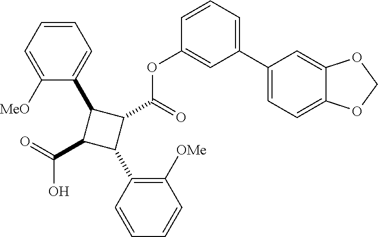 |
| 1e | 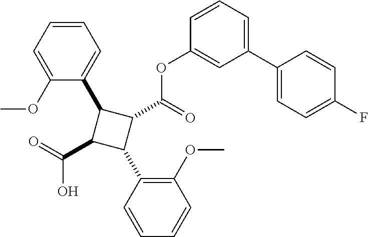 |
| 1f | 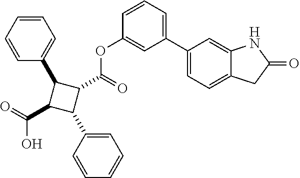 |
| 1g | 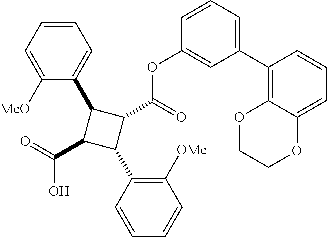 |
| 1h | 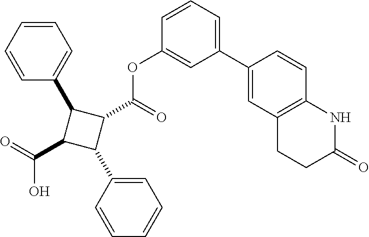 |
| 1i | 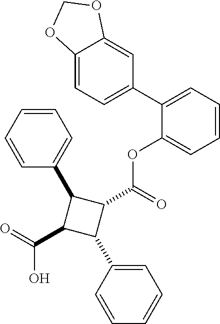 |
| 1j | 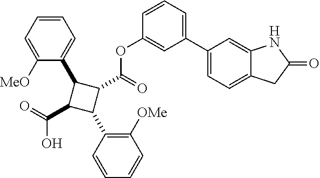 |
| 1k | 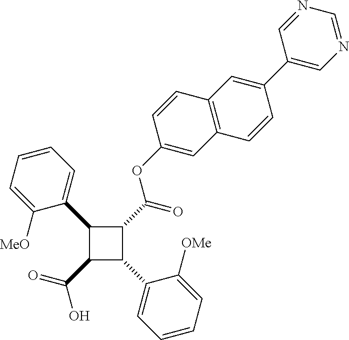 [SMILES: COc1ccccc1[C@@H]1[C@H](C(=O)O)[C@@H](c2ccccc2OC)[C@H]1C(=O)Oc1ccc2cc(-c3cncnc3)ccc2c1] |
| 1l | 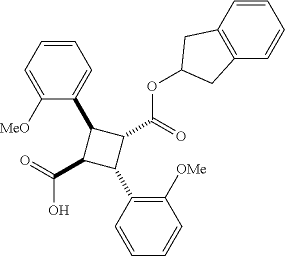 |
| 1m | 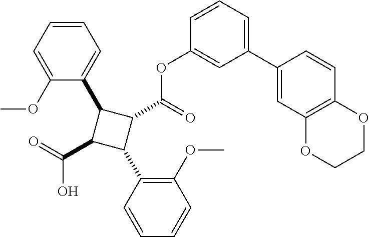 |
| 1n | 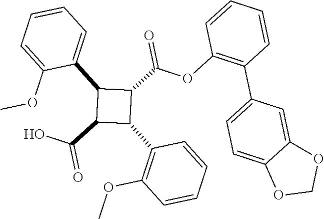 |
| 1o | 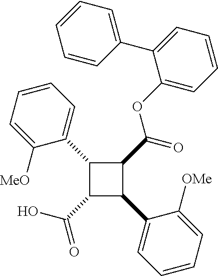 [SMILES: COc1ccccc1[C@H]1[C@H](C(=O)O)[C@H](c2ccccc2OC)[C@H]1C(=O)Oc1ccccc1-c1ccccc1] |
| 1p | 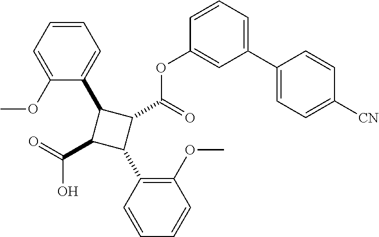 |
| 1q | 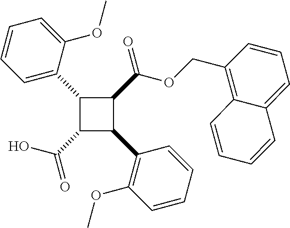 [SMILES: COc1ccccc1[C@H]1[C@H](C(=O)O)[C@H](c2ccccc2OC)[C@H]1C(=O)OCc1cccc2ccccc12] |
| 1r | 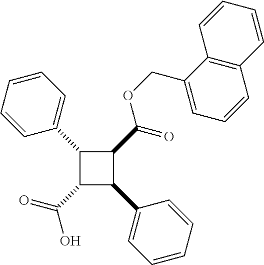 [SMILES: O=C(O)[C@H]1[C@H](c2ccccc2)[C@H](C(=O)OCc2cccc3ccccc23)[C@H]1c1ccccc1] |
| 1s | 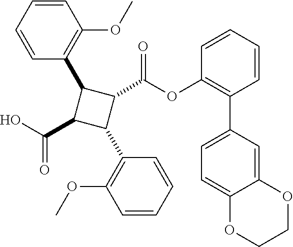 |
| 1t | 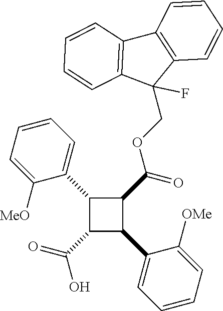 [SMILES: COc1ccccc1[C@H]1[C@H](C(=O)O)[C@H](c2ccccc2OC)[C@H]1C(=O)OCC1(F)c2ccccc2-c2ccccc21] |
| 1u | 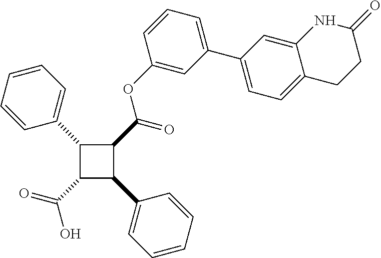 [SMILES: O=C1CCc2ccc(-c3cccc(OC(=O)[C@H]4[C@@H](c5ccccc5)[C@H](C(=O)O)[C@@H]4c4ccccc4)c3)cc2N1] |
| 1v | 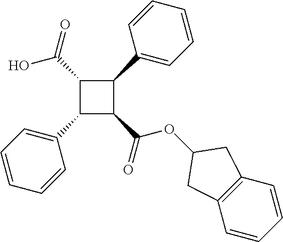 [SMILES: O=C(O)[C@H]1[C@@H](c2ccccc2)[C@H](C(=O)OC2Cc3ccccc3C2)[C@@H]1c1ccccc1] |
| 1w | 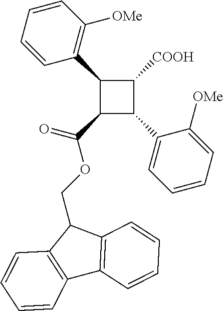 [SMILES: COc1ccccc1[C@H]1[C@H](C(=O)O)[C@H](c2ccccc2OC)[C@H]1C(=O)OCC1c2ccccc2-c2ccccc21] |
| 1x | 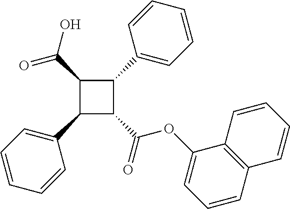 [SMILES: O=C(O)[C@H]1[C@H](c2ccccc2)[C@H](C(=O)Oc2cccc3ccccc23)[C@H]1c1ccccc1] |
| 1y | 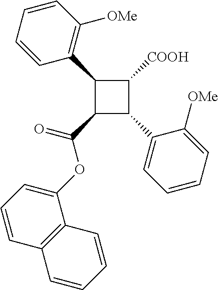 [SMILES: COc1ccccc1[C@H]1[C@H](C(=O)O)[C@H](c2ccccc2OC)[C@H]1C(=O)Oc1cccc2ccccc12] |
| 1z | 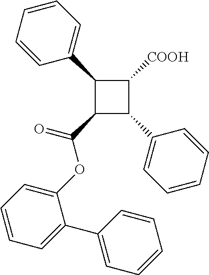 [SMILES: O=C(O)[C@H]1[C@H](c2ccccc2)[C@H](C(=O)Oc2ccccc2-c2ccccc2)[C@H]1c1ccccc1] |
| 2a | 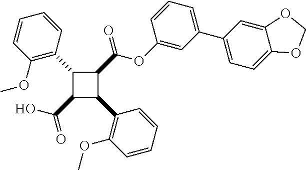 [SMILES: COc1ccccc1[C@H]1[C@H](C(=O)O)[C@H](c2ccccc2OC)[C@@H]1C(=O)Oc1cccc(-c2ccc3c(c2)OCO3)c1] |
| 2b | 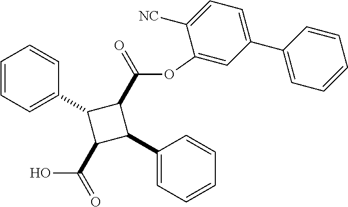 |
| 2c | 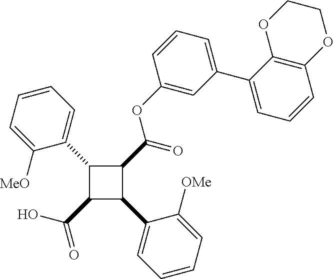 [SMILES: COc1ccccc1[C@H]1[C@H](C(=O)O)[C@H](c2ccccc2OC)[C@@H]1C(=O)Oc1cccc(-c2cccc3c2OCCO3)c1] |
| 2d | 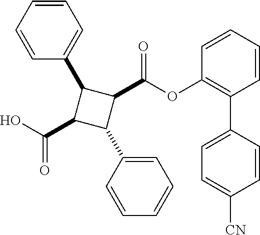 |
| 2e | 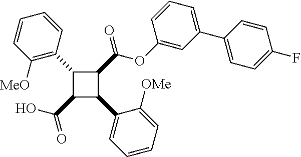 [SMILES: COc1ccccc1[C@H]1[C@H](C(=O)O)[C@H](c2ccccc2OC)[C@@H]1C(=O)Oc1cccc(-c2ccc(F)cc2)c1] |
| 2f | 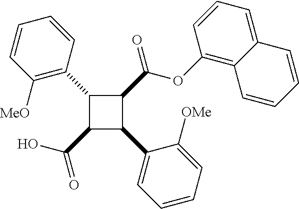 [SMILES: COc1ccccc1[C@H]1[C@H](C(=O)O)[C@H](c2ccccc2OC)[C@@H]1C(=O)Oc1cccc2ccccc12] |
| 2g | 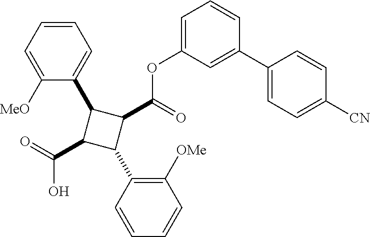 |
| 2h | 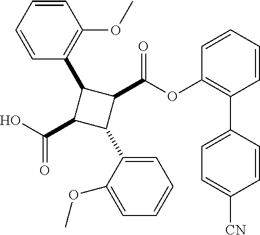 |
| 2i | 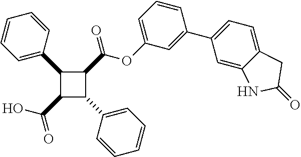 [SMILES: O=C1Cc2ccc(-c3cccc(OC(=O)[C@H]4[C@H](c5ccccc5)[C@@H](C(=O)O)[C@H]4c4ccccc4)c3)cc2N1] |
| 2j | 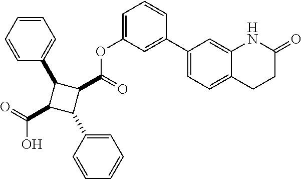 |
| 2k | 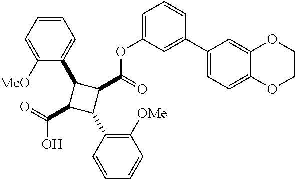 |
| 2l | 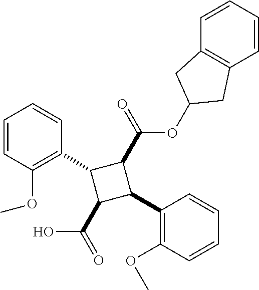 |
| 2m | 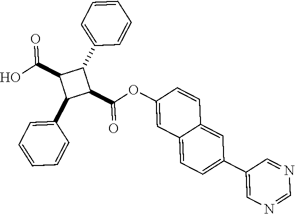 [SMILES: O=C(O)[C@H]1[C@H](c2ccccc2)[C@@H](C(=O)Oc2ccc3cc(-c4cncnc4)ccc3c2)[C@H]1c1ccccc1] |
| 2n | 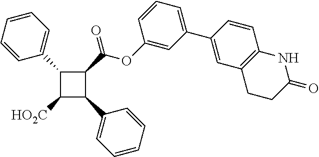 [SMILES: O=C1CCc2cc(-c3cccc(OC(=O)[C@H]4[C@@H](c5ccccc5)[C@@H](C(=O)O)[C@@H]4c4ccccc4)c3)ccc2N1] |
| 3a | 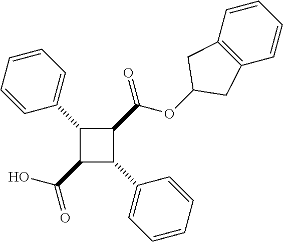 [SMILES: O=C(O)[C@@H]1[C@@H](c2ccccc2)[C@H](C(=O)OC2Cc3ccccc3C2)[C@H]1c1ccccc1] |
| 3b | 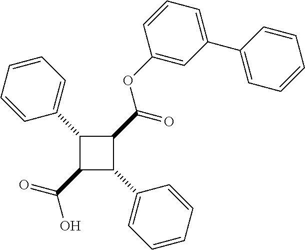 [SMILES: O=C(O)[C@@H]1[C@@H](c2ccccc2)[C@H](C(=O)Oc2cccc(-c3ccccc3)c2)[C@H]1c1ccccc1] |
| 3c | 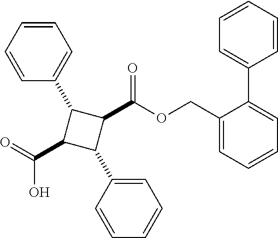 |
| 3d | 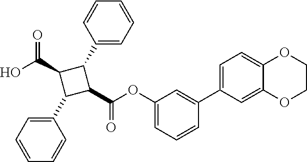 |
| 3e | 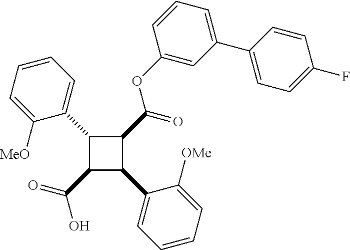 [SMILES: COc1ccccc1[C@H]1[C@H](C(=O)O)[C@H](c2ccccc2OC)[C@@H]1C(=O)Oc1cccc(-c2ccc(F)cc2)c1] |
| 3f | 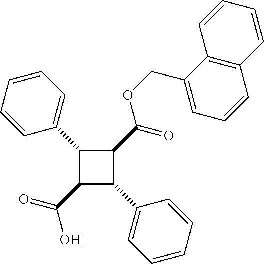 [SMILES: O=C(O)[C@@H]1[C@@H](c2ccccc2)[C@H](C(=O)OCc2cccc3ccccc23)[C@H]1c1ccccc1] |
| 3g | 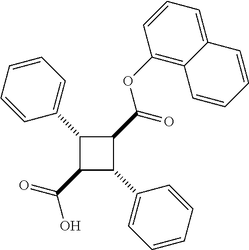 [SMILES: O=C(O)[C@@H]1[C@@H](c2ccccc2)[C@H](C(=O)Oc2cccc3ccccc23)[C@H]1c1ccccc1] |

In some embodiments, the compound is the (S,S) enantiomer. In some embodiments, the compound is the (R,R) enantiomer.

In some embodiments, the composition comprises a mixture of enantiomers enriched in (S,S) enantiomer. In some embodiments, the composition comprises a mixture of enantiomers enriched in (R,R) enantiomer.

In some embodiments, the method wherein the compound is the (S,S) enantiomer. In some embodiments, the method wherein the compound is the (R,R) enantiomer.

In some embodiments of the method of inhibiting the activity of a Fatty Acid Binding Protein (FABP), wherein the compound inhibits binding of an FABP ligand to the FABP.

In some embodiments of the method of inhibiting the activity of a Fatty Acid Binding Protein (FABP), wherein the FABP ligand is an endocannabinoid ligand.

In some embodiments of the method of inhibiting the activity of a Fatty Acid Binding Protein (FABP), wherein the FABP ligand is anandamide (AEA) or 2-arachidonoylglycerol (2-AG).

In some embodiments, a method of treating a neurological disorder which affects at least one of movement, memory, mood, appetite, nociception, endocrine regulation, thermoregulation, sensory perception, or cognitive functions.

In some embodiments, a method of treating a neurological disorder associated with drug addiction, depression, compulsive behavior, neuropathic pain, or a movement disorder.

In some embodiments, a method of treating drug addiction, depression, compulsive behavior, neuropathic pain, inflammatory pain, or a movement disorder.

In some embodiments, a method of treating pain, neuropathic pain, or inflammatory pain.

In some embodiments, a method of treating a subject afflicted with a neurological disorder which affects at least one of movement, memory, mood, appetite, nociception, endocrine regulation, thermoregulation, sensory perception, or cognitive functions, comprising administering to the subject a compound of the present application.

In some embodiments, a method of treating a subject afflicted with a neurological disorder associated with drug addiction, depression, compulsive behavior, neuropathic pain, or a movement disorder, comprising administering to the subject a compound of the present application.

In some embodiments, a method of treating a subject afflicted with drug addiction, depression, compulsive behavior, neuropathic pain, inflammatory pain, or a movement disorder, comprising administering to the subject a compound of the present application.

In some embodiments, a method of treating a subject afflicted with pain, neuropathic pain, or inflammatory pain, comprising administering to the subject a compound of the present application.

As used herein, the term “endocannabinoid” includes any molecule that activates cannabinoid receptors. Examples of such receptors are CB1 and CB2. Examples of endocannabinoids are arachidonoyl ethanolamide (AEA) and 2-arachidonoyl glycerol (2-AG).

As used herein, the term “fatty acid binding protein” or “FABP” refers to fatty acid binding proteins (FABPs) that function as intracellular carriers that shuttle cannabinoids (and by extension fatty acid amides (FAAs)) to FAAH where cannabinoids are hydrolyzed and degraded. Further, uptake of endocannabinoids (and by extension FAAs) by the cell and the subsequent hydrolysis of endocannabinoids (and by extension FAAs) are enhanced by FABPs, and inhibiting the interaction of endocannabinoids (and by extension FAAs) with FABPs reduces endocannabinoid (and by extension FAA) uptake and hydrolysis. FABPS include, for example, fatty acid binding protein 1 (FABP 1), fatty acid binding protein 2 (FABP 2), fatty acid binding protein 3 (FABP 3), fatty acid binding protein 4 (FABP 4), fatty acid binding protein 5 (FABP 5), fatty acid binding protein 6 (FABP 6), fatty acid binding protein 7 (FABP 7), fatty acid binding protein 8 (FABP 8), fatty acid binding protein 9 (FABP 9), fatty acid binding protein 10 (FABP 10), fatty acid binding protein 11 (FABP 11), fatty acid binding protein 5-like (FABP 5-like 1), fatty acid binding protein 5-like 2 (FABP 5-like 2), fatty acid binding protein 5-like 3 (FABP 5-like 3), fatty acid binding protein 5-like 4 (FABP 5-like 4), fatty acid binding protein 5-like 5 (FABP 5-like 5), fatty acid binding protein 5-like 6 (FABP 5-like 6), and fatty acid binding protein 5-like 7 (FABP 5-like 7) (see Chmurzynska et al. 2006 and PCT International Application Publication No. WO 2010/083532 A1, the contents of each of which are hereby incorporated by reference).

As used herein, the term “therapeutic agent” refers to any agent used to treat a disease or that provides a beneficial therapeutic effect to a subject.

As used herein, the phrase “inhibits the interaction” is employed herein to refer to any disruption, partial or total, of the natural effect of FABPs on the metabolism of endocannabinoids.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

As used herein, “treating” means reducing, slowing, stopping, preventing, reversing, or in any way improving the progression of a disease or disorder or a symptom of the disease or disorder.

In some embodiments, the compounds of the present invention include all hydrates, solvates, and complexes of the compounds used by this invention.

In some embodiments, if a chiral center or another form of an isomeric center is present in a compound of the present invention, all forms of such isomer or isomers, including enantiomers and diastereomers, are intended to be covered herein.

In some embodiments, if a chiral center or another form of an isomeric center is present in a compound of the present invention, only enantiomeric forms are intended to be covered herein.

Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. A method by which to obtain the individual enantiomers is described in WO 2014/015276, published Jan. 23, 2014, the contents of which are hereby incorporated by reference.

As used herein, “enantiomers” are non-identical, non-superimposible mirror images of each other. For any given chiral compound, only one pair of enantiomers exists. The enantiomers can be separated using known techniques, including those described in Pure and Applied Chemistry 69, 1469-1474, (1997) IUPAC.

In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, cycloalkyl, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include individual groups each having 1, 2, . . . , n−1 or n carbons in a linear or branched arrangement. For example, C₁-C₆, as in “C₁-C₆ alkyl” is defined to include individual groups each having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl) and may be unsubstituted or substituted. The “cycloalkyl” may be substituted with a phenyl or a fused benzo group including, but not limited to, 2-indanyl, 9-fluorenyl, or 9-fluoro-9-fluorenyl.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “alkylaryl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethyl-phenylmethyl), 1-phenylethyl, 2-phenylethyl, 3-phenylpropyl, 2-phenylpropyl, (1,1′-biphenyl)methyl, 1-naphthylmethyl and the like.

The term “alkylcycloalkyl” refers to alkyl groups as described above wherein one or more bonds to hydrogen contained therein are replaced by a bond to a cycloalkyl group as described above. It is understood that an “alkylcycloalkyl” group is connected to a core molecule through a bond from the alkyl group and that the cycloalkyl group acts as a substituent on the alkyl group. Examples of arylalkyl moieties include, but are not limited to, (9-fluorenyl)methyl, (9-fluoro-9-fluorenyl)methyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or polycyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different.

The term “tolyl” refers to one of the three CH₃C₆H₄— isomeric groups derived from toluene.

The term “naphthalene” refers to a bicyclic aromatic hydrocarbon consisting of a fused pair of benzene rings.

The term “2-(1,1′-biphenyl)” refers to the structure:

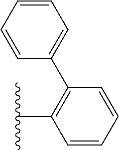 [SMILES: *c1ccccc1-c1ccccc1]

The term “3-(1,1′-biphenyl)” refers to the structure:

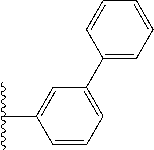 [SMILES: *c1cccc(-c2ccccc2)c1]

The term “4-(1,1′-biphenyl)” refers to the structure:

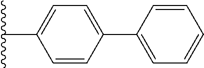 [SMILES: *c1ccc(-c2ccccc2)cc1]

The term “2-(2′-hydroxy-1,1′-biphenyl)” refers to the structure:

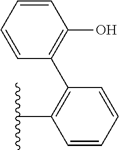 [SMILES: *c1ccccc1-c1ccccc1O]

The term “1-naphthalene-6-acetamide” refers to the structure:

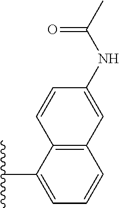 [SMILES: *c1cccc2cc(NC(C)=O)ccc12]

The term “1-naphthalene-5-ethyne” refers to the structure:

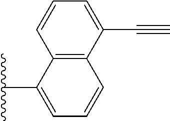 [SMILES: *c1cccc2c(C#C)cccc12]

The term “3-[1-(3,6,9-trioxa-dodecanyl)-1,2,3-triazol-4-yl]phenyl” refers to the structure:

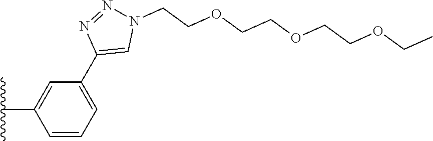 [SMILES: *c1cccc(-c2cn(CCOCCOCCOCC)nn2)c1]

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity. The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Materials and Methods

Those having ordinary skill in the art of organic synthesis will appreciate that modifications to general procedures and synthetic routes contained in this application can be used to yield additional derivatives and structurally diverse compounds. Suitable organic transformations are described in in March's Advanced Organic Chemistry: Reactions, Mechanisms, and Structure (Wiley-Interscience; 6th edition, 2007), the content of which is hereby incorporated by reference.

Analytical Methods.

NMR spectra were recorded on either a Bruker Ascend 700 spectrometer operating at 700 MHz for ¹H acquisitions and 175 MHz for ¹³C acquisitions, a Bruker 500 Advance spectrometer operating at 500 MHz and 125 MHz for ¹H and ¹³C acquisitions, respectively, a Bruker 400 Nanobay spectrometer operating at 400 MHz, 100 MHz, and 376 MHz for ¹H, ¹³C, and ¹⁹F acquisitions, respectively. Chemical shifts were referenced to the residual proton solvent peaks (¹H: CDCl₃, δ 7.26; (CD₃)₂SO, δ 2.50; CD₃OD, δ 3.31; CD₃CN, δ 1.94), solvent ¹³C signals (CDCl₃, δ 77.16; (CD₃)₂SO, δ 39.52; CD₃OD, δ 49.00). Signals are listed in ppm, and multiplicity identified as s=singlet, br=broad, d=doublet, t=triplet, q=quartet, m=multiplet; coupling constants in Hz; integration. High-resolution mass spectra were performed at Mass Spectrometry Services at the Univ. of Illinois at Urbana-Champaign and were obtained using Waters Q-TOF Ultima ESI mass spectrometer. Concentration under reduced pressure was performed by rotary evaporation at 25-30° C. at appropriate pressure.

Materials.

All air- and moisture-insensitive reactions were carried out under an ambient atmosphere, magnetically stirred, and monitored by thin layer chromatography (TLC) using Agela Technologies TLC plates pre-coated with 250 μm thickness silica gel 60 F254 plates and visualized by fluorescence quenching under UV light. Flash chromatography was performed on SiliaFlash® Silica Gel 40-63 μm 60 Å particle size using a forced flow of eluent at 0.3-0.5 bar pressure.1 All air- and moisture-sensitive manipulations were performed using oven-dried glassware, including standard Schlenk and glovebox techniques under an atmosphere of nitrogen. Diethyl ether and THF were distilled from deep purple sodium benzophenone ketyl. Methylene chloride, chloroform and acetonitrile were dried over CaH₂ and distilled. Methylene chloride was degassed via three freeze-pump-thaw cycles. All other chemicals were used as received. All deuterated solvents were purchased from Cambridge Isotope Laboratories.

### Example 1. Synthesis of α-Truxillic Acid Monoesters

### (±)-α-3-(2-cyano-5-phenyl)phenoxycarbonyl)-2,4-diphenylcyclobutane-1-carboxylic acid 1a

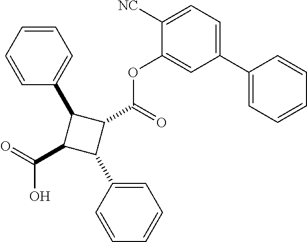

The synthesis commenced by the chlorination of 2,2′-dimethoxy truxillic acid (198 mg, 0.55 mmol) with thionyl chloride (4 mL) and a catalytic amount of DMF under reflux in inert atmosphere for 3 hours. The mixture was dried in vacuo and the resulting diacid chloride was dissolved in dry THF (10 mL). To this mixture was added pyridine (0.5 mL), followed by 2-cyano-5-phenylphenol (61 mg, 0.44 mmol). The reaction mixture was stirred under nitrogen for 18 hours and then dried in vacuo. The resulting crude product was purified by a column chromatography first on silica gel using hexane/ethyl acetate (1/1) as eluent, followed by that on a C18 silica using acetonitrile/water (1/1) to give the titled compound (70 mg, 45% yield) as off-white solid: m.p. 196 −198° C.; ¹H NMR (400 MHz, acetone-d₆) δ 7.85 (d, J=8.1 Hz, 1H), 7.69 (dd, J=8.1, 1.7 Hz, 1H), 7.66-7.61 (m, 2H), 7.59-7.45 (m, 9H), 7.42-7.35 (m, 3H), 7.33-7.26 (m, 1H), 6.23 (d, J=1.7 Hz, 1H), 4.75 (dd, J=10.8, 7.1 Hz, 1H), 4.67 (dd, J=10.7, 6.9 Hz, 1H), 4.44 (ddd, J=10.8, 6.9, 1.1 Hz, 1H), 4.20 (ddd, J=10.8, 7.1, 1.1 Hz, 1H); ¹³C NMR (176 MHz, acetone-d₆) δ 172.9, 170.7, 153.3, 147.8, 140.0, 139.8, 138.7, 134.4, 130.8, 130.0, 129.6, 129.2, 129.1, 128.6, 128.4, 128.1, 127.9, 125.8, 121.9, 115.7, 106.3, 47.5, 46.8, 42.9, 42.1; HRMS (ESI-TOF)⁺ m/z calculated for C₃₁H₂₇N₂O₄⁺ [M+NH₄]⁺ 491.1965, found 491.1976 (Δ=2.23 ppm).

### (±)-α-3-(2-cyano-5-phenyl)phenoxycarbonyl)-2,4-di (2-methoxyphenyl)cyclobutane-1-carboxylic acid 1b

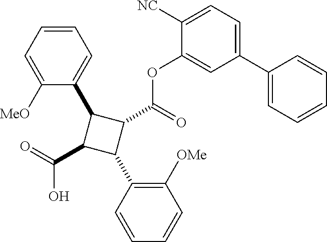

The same procedure as that for 1a was used. Off-white solid; m.p. 169-170° C.; ¹H NMR (500 MHz, acetone-d₆) δ 10.55 (bs, 1H), 7.85 (d, J=8.1 Hz, 1H), 7.68 (dd, J=8.1, 1.5 Hz, 1H), 7.60-7.45 (m, 7H), 7.36 (t, J=7.8 Hz, 1H), 7.27 (t, J=7.8 Hz, 1H), 7.08 (m, 2H), 6.99 (m, 2H), 6.20 (d, J=1.5 Hz, 1H), 4.92 (dd, J=10.4, 8.4, 1H), 4.81 (dd, J=10.5, 6.4, 1H), 4.29 (dd, J=10.5, 6.4, 1H), 4.20 (dd, J=10.4, 8.3 Hz, 1H), 3.88 (s, 3H), 3.87 (s, 3H); ¹³C NMR (125 MHz, Acetone-d₆) δ 173.2, 171.2, 158.9, 158.6, 153.5, 147.8, 138.7, 134.4, 129.98, 129.96, 129.6, 129.1, 128.5, 128.2, 128.1, 125.6, 121.8, 121.3, 121.1, 115.7, 111.4, 111.3, 106.4, 56.0, 55.7, 46.3, 44.7, 38.0, 37.5; HRMS (ESI⁻) m/z calcd. for C₃₃H₂₆NO₆⁻ [M−H]⁻ 532.1766, found 532.1757 (A 1.7 ppm).

### (±)-α-3-[3-(benzo[d][1,3]dioxol-5-yl)phanoxycarbonyl]-2,4-di (2-methoxyphenyl)cyclobutane-1,3-dicarboxylic acid 1c

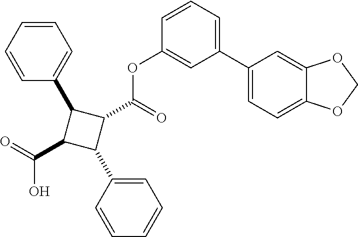

The same procedure as that for 1a was used. Off-white solid; mp.>220° C.; ¹H NMR (500 MHz, DMSO-d₆) δ 12.22 (s, 1H), 7.52 (d, J=7.4, 2H) 7.49-7.33 (m, 7H), 7.33-7.26 (m, 2H), 7.07 (d, J=1.7 Hz, 1H), 7.03 (d, J=8.1 Hz, 1H), 6.97 (dd, J=8.1, 1.7 Hz, 1H), 6.44 (dd, J=8.0, 1.5 Hz, 1H), 6.35 (t, J=1.8 Hz, 1H), 6.09 (s, 2H), 4.55 (dd, J=10.8, 7.5 Hz, 1H), 4.46 (dd, J=10.7, 6.7 Hz, 1H), 4.22 (dd, J=10.8, 6.7 Hz, 1H), 3.98 (dd, J=10.7, 7.5 Hz, 1H). ¹³C NMR (175 MHz, acetone-d₆) δ 172.1, 170.5, 151.2, 148.4, 147.6, 141.8, 139.3, 139.2, 133.9, 129.4, 128.5, 128.2, 128.2, 127.8, 127.3, 126.9, 123.7, 120.5, 120.1, 119.8, 108.4, 107.1, 101.4, 46.6, 46.0, 41.9, 41.3.

HRMS (ESI-TOF) m/z calcd. for C₃₁H₂₅O₆⁺ [M+H]⁺ 493.1573, found 493.1644 (Δ=0.71 ppm).

### (±)-α-3-[3-(benzo[d][1,3]dioxol-5-yl)phenoxycarbonyl]-2,4-di (2-methoxyphenyl)cyclobutane-1-dicarboxylic acid 1d

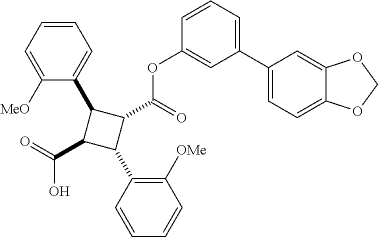

The same procedure as that for 1a was used. Off-white solid; m.p. 187° C. (decomp.); ¹H NMR (500 MHz, acetone-d₆) δ 10.50 (s, 1H), 7.47 (m, 2H), 7.37 (m, 2H), 7.35-7.25 (m, 2H) 7.10-6.95 (m, 7H), 6.54 (dd, J=8.0, 1.4, 1H), 6.44 (dd, J=1.9, 1.9, 1H), 6.08 (s, 2H), 4.87 (dd, J=10.6, 7.9, 1H), 4.78 (dd, J=10.5, 6.7, 1H), 4.25 (dd, J=10.6, 6.8, 1H), 4.13 (dd, J=10.4, 8, 1H), 3.89 (s, 3H), 3.88 (s, 3H); ¹³C NMR (125 MHz, acetone-d₆) δ 172.5, 170.9, 158.0, 157.7, 151.4, 148.4, 147.6, 141.8, 134.0, 129.4, 128.5, 128.1, 127.6, 127.5, 127.44, 127.42, 123.6, 120.5, 120.4, 120.2, 120.1, 119.7, 110.5, 110.4, 108.4, 107.2, 101.4, 55.1, 54.9, 45.2, 44.2, 36.9, 36.3; HRMS (ESI-TOF) m/z calcd. for C₃₃H₂₈O₈[M−H]⁺ 552.17842, found 552.18642 (Δ 0.58 ppm).

### (±)-α-2,4-di (2-methoxyphenyl)-3-[3-(4-fluorophenyl)phenoxycarbonyl]cyclobutane-1-carboxylic acid 1e

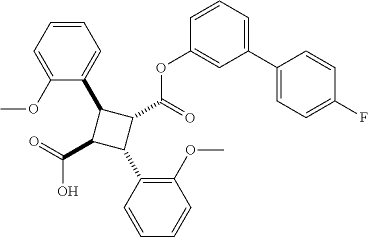

The same procedure as that for 1a was used, but without purification on C18 silica and with washing of the product with methanol. While solid; m.p. 191-193° C.; ¹H NMR (500 MHz, acetone-d₆) δ 10.41 (s, 1H), 7.58 (dd, J=8.6, 5.4 Hz, 2H), 7.49-7.16 (m, 8H), 7.16-6.87 (m, 4H), 6.59 (d, J=8.0 Hz, 1H), 6.48 (s, 1H), 4.88 (dd, J=10.4, 8.0 Hz, 1H), 4.78 (dd, J=10.6, 6.8 Hz, 1H), 4.26 (dd, J=10.7, 6.7 Hz, 1H), 4.13 (dd, J=10.4, 8.0 Hz, 1H), 3.89 (s, 3H), 3.88 (s, 3H); ¹³C NMR (125 MHz, acetone-d₆) δ 172.6, 170.9, 162.6 (d, J=245.0 Hz), 158.0, 157.8, 151.4, 141.1, 136.1 (d, J=3.2 Hz), 129.6, 128.8 (d, J=8.2 Hz), 128.5, 128.1, 127.6, 127.5, 127.47, 127.4, 123.8, 120.5, 120.4, 120.2, 120.0, 115.5 (d, J=21.6 Hz), 110.5, 110.4, 55.1, 54.9, 45.2, 44.2, 36.9, 36.3; HRMS (ESI-TOF) m/z calcd. for C₃₂H₂₈FO₆ [M+H]⁺ 527.1873, found 527.1864 (A 1.57 ppm).

### (±)-α-3-[3-(2-oxoindolin-6-yl)phenoxycarbonyl]-2,4-diphenylcyclobutane-1-carboxylic acid 1f

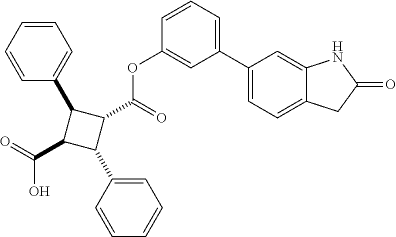

N-Ethyl-N′-(3-dimethylaminopropyl) carbodiimide hydrochloride (EDC-HCl) (261 mg, 1.36 mmol) and 4-dimethylaminopyridine (166 mg, 1.36 mmol) were added to a stirred solution of α-truxillic acid (295 mg, 0.99 mmol) and 6-(3-hydroxyphenyl)indolin-2-one (280 mg, 1.24 mmol) in anhydrous THF (20 mL). The reaction mixture was stirred at room temperature for 20 hours. Then, the reaction was quenched with water (20 mL). The pH of the reaction mixture was adjusted to 5 with 5% solution of NaH₂PO₄ and 1M HCl, and dichloromethane (200 mL) was added. The resulting suspension was filtered to give a pink solid (190 mg). The ¹H NMR of this pink solid indicated that it was a ca. 1:1 mixture of monoester (title compound) and diester. Since the R_{f} values of these two products were close, this pink solid was not subjected to purification this time. The filtrate was extracted 2 twice with dichloromethane (100 mL) and the combined organic layers were washed with brine, dried over anhydrous magnesium sulfate and filtered. The filtrate was concentrated under reduced pressure to give a crude product. The resulting crude product was purified by automatic flash chromatography (Yamazen), using two 16 g silica gel columns in tandem and a 1:1 mixture of hexanes and ethyl acetate as eluant to give the title compound (45 mg, 9% yield, not optimized) as a off-white solid: mp>230° C.; ¹H NMR (500 MHz, DMSO-d₆) δ 12.20 (bs, 1H), 10.59 (s, 1H), 7.52-7.27 (m, 13H), 7.05 (dd, J=7.6, 1.3 Hz, 1H), 6.84 (s, 1H), 6.55 (dd, J=7.9, 1.2 Hz, 1H), 6.24 (bs, 1H), 4.55 (dd, J=10.7, 7.5 Hz, 1H), 4.46 (dd, J=10.7, 6.8 Hz, 1H), 4.23 (dd, J=10.9, 6.8 Hz, 1H), 3.98 (dd, J=10.9, 7.5 Hz, 1H), 3.53 (s, 2H); ¹³C NMR (125 MHz, DMSO-d6) δ 176.6, 172.6, 170.6, 150.5, 144.4, 141.6, 139.0, 138.99, 138.4, 130.2, 129.7, 128.5, 128.2, 128.0, 127.8, 127.3, 126.8, 125.6, 124.7, 123.9, 119.8, 119.5, 107.2, 46.1, 45.6, 41.4, 40.6, 35.7; HRMS (ESI-TOF) m/z calcd. for C₃₂H₂₆NO₅⁺, [M+H]⁺ 504.1805, found 504.1816 (Δ=2.2 ppm).

### (±)-α-3-[3-(2,3-dihydrobenzo[b][1,4]dioxin-5-yl)phenoxycarbonyl]-2,4-di(2-methoxyphanyl)cyclobutane-1-carboxylic acid 1g

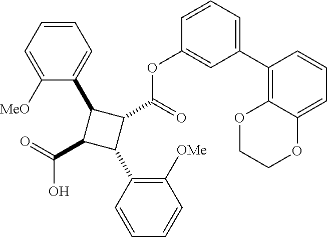

N-Ethyl-N′-(3-dimethylaminopropyl) carbodiimide hydrochloride (EDC-HCl) (242 mg, 1.26 mmol) and 4-dimethylaminopyridine (DMAP) (153 mg, 1.26 mmol) were added to a stirred solution of a-di(2-methoxy)truxillic acid (410 mg, 1.15 mmol) and 3-(2,3-dihydrobenzo[b][1,4]dioxin-5-yl)phenol (210 mg, 0.92 mmol) in anhydrous CH₂Cl₂ (20 mL). The reaction mixture was stirred at room temperature for 20 hours and then the reaction was quenched with water (20 mL). The pH of the reaction mixture was adjusted to 5 with 5% aqueous solution of NaH₂PO₄ and 1M HCl. Then, the reaction mixture was extracted with dichloromethane (2×70 mL) and ethyl acetate (30 ml). The combined organic layers were washed with brine and dried over anhydrous magnesium sulfate and filtered. The filtrate was concentrated under reduced pressure to give a crude product, which was purified by flash chromatography on silica gel, using hexanes/ethyl acetate (3/1) as eluent to give the title compound (152 mg, 29% yield, not optimized) as a colorless solid: m.p. 199-200° C.; ¹H NMR (500 MHz, acetone-d₆) δ 7.44 (t, J=9.0 Hz, 2H), 7.33-7.24 (m, 4H), 7.06-6.97 (m, 4H), 6.91 (t, J=7.8 Hz, 1H), 6.85 (dd, J=8.0, 1.6 Hz, 1H), 6.78 (dd, J=7.4, 1.6 Hz, 1H), 6.59 (t, J=1.8 Hz, 1H), 6.48 (dd, J=8.0, 1.2 Hz, 1H), 4.82 (dd, J=10.5, 7.8 Hz, 1H), 4.75 (dd, J=10.5, 6.9 Hz, 1H), 4.32-4.23 (m, 5H), 4.10 (dd, J=10.4, 7.8 Hz, 1H), 3.88 (s, 3H), 3.87 (s, 3H); ¹³C NMR (125 MHz, acetone-d₆) δ 172.7, 170.9, 157.9, 157.7, 150.6, 144.1, 140.8, 139.0, 129.5, 128.4, 128.3, 128.0, 127.5, 127.46, 127.44, 127.41, 126.5, 122.4, 120.8, 120.4, 120.2, 120.1, 116.7, 110.4, 64.2, 64.1, 55.0, 54.9, 45.2, 44.3, 37.0, 36.5;

HRMS (ESI-TOF) m/z calcd. for C₃₄H₂₉O₈⁻ [M−H]⁻ 565.1868, found 565.1873 (Δ=0.9 ppm).

### (±)-α-2,4-diphenyl-3-[3-(2-oxo-1,2,3,4-tetrahydroquinolin-6-yl)phenoxycarbonyl]cyclobutane-1-carboxylic acid 1h

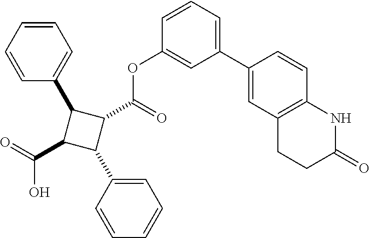

The same procedure as that for 1g was used, except for using 1-3% methanol in dichromomethane was used as eluent. Off-white solid; m.p.>230° C.; ¹H NMR (500 MHz, acetone-d₆) δ 9.17 (s, 1H), 7.70-7.21 (m, 14H), 7.04 (d, J=8.8 Hz, 1H), 6.52 (dd, J=8.1, 2.3 Hz, 1H), 6.48 (t, J=2.0 Hz, 1H), 4.66 (dd, J=10.9, 7.2 Hz, 1H), 4.60 (dd, J=10.8, 7.0 Hz, 1H), 4.31 (dd, J=10.9, 7.0 Hz, 1H), 4.13 (dd, J=10.8, 7.2 Hz, 1H), 3.08 (t, J=7.5 Hz, 2H), 2.58 (t, J=7.5 Hz, 2H); ¹³C NMR (175 MHz, DMSO-d₆) δ 173.1, 171.1, 170.7, 151.1, 141.5, 139.60, 139.58, 138.6 (C_{Ar}C_{Ar}), 132.9, 130.1, 128.9, 128.7, 128.6, 128.3, 127.6, 127.2, 126.4, 125.8, 124.6, 123.8, 120.3, 119.4, 115.9, 46.6, 41.1, 40.5, 30.8, 25.3; HRMS (ESI-TOF) m/z calculated for C₃₃H₂₈NO₅⁻ [M+H]⁺ 518.1962, found 518.1948, (Δ=2.7 ppm).

### (±)-α-3-[2-(benzo[d][1,3]dioxol-5-yl)phenoxycarbonyl]-2,4-di (2-dimethoxyphenyl)cyclobutane-1-dicarboxylic acid 1i

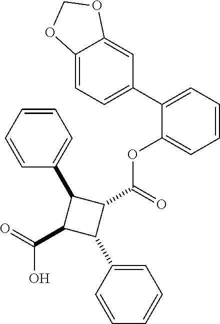

The same procedure as that for 1g was used. Off-white solid; m.p. 175-177° C.; ¹H NMR (500 MHz, DMSO-d₆) δ 12.29 (bs, 1H), 7.70-7.08 (m, 13H), 6.91 (d, J=8.0 Hz, 1H), 6.86 (d, J=1.7 Hz, 1H), 6.78 (dd, J=8.0, 1.7 Hz, 1H), 6.03 (d, J=5.9 Hz, 2H), 5.93 (d, J=8.0 Hz, 1H), 4.39 (dd, J=10.6, 6.4 Hz, 1H), 4.29 (dd, J=10.6, 7.7 Hz, 1H), 4.00 (dd, J=10.6, 7.7 Hz, 1H), 3.90 (dd, J=10.6, 6.4 Hz, 1H); ¹³C NMR (175 MHz, acetone-d₆) δ 172.4, 169.9, 147.7, 147.2, 139.3, 139.2, 134.5, 131.3, 130.3, 128.5, 128.2, 128.0, 127.9, 127.3, 127.1, 126.7, 126.1, 122.6, 122.4, 109.0, 108.1, 101.3, 46.3, 46.0, 41.8, 41.1; ¹³C NMR (125 MHz, DMSO-d6) δ 173.4, 170.4, 147.8, 147.5, 147.2, 139.4, 139.3, 134.3, 131.0, 130.9, 128.9, 128.6, 128.3, 127.6, 127.5, 127.2, 126.9, 122.65, 122.62, 109.3, 108.7, 101.6, 46.3, 41.7, 40.9; HRMS (ESI-TOF) m/z calcd. for C₃₁H₂₅O₆⁺ [M+H]⁺ 493.1646, found 493.1661 (A 3.04 ppm).

### (±)-α-2,4-di (2-methoxyphenyl)-3-[3-(2-oxoindolin-6-yl)phenoxycarbonyl]cyclobutane-1-carboxylic acid 1j

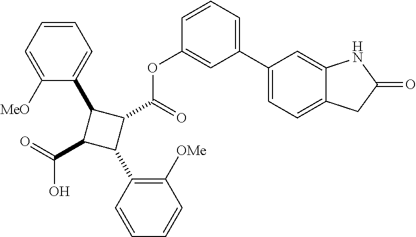

The same procedure as that for 1g was used except for using methanol/dichloromethane (5-80% gradient) as eluent, and washing the product with ethanol. White solid; m.p. 211-212° C.; ¹H NMR (500 MHz, acetone) δ 9.50 (s, 1H), 7.45 (d, J=7.5 Hz, 2H), 7.42-7.31 (m, 4H), 7.26 (m, 1H), 7.11 (dd, J=7.7, 1.7 Hz, 1H), 7.07 (m, 2H), 6.99 (m, 3H), 6.60 (d, J=8 Hz, 1H), 6.47 (t, J=1.9 Hz, 1H), 4.85 (dd, J=10.6, 7.5 Hz 1H), 4.77 (dd, J=10.4, 6.7 Hz, 1H), 4.26 (dd, J=10.9, 6.7 Hz, 1H), 4.11 (dd, J=10.7, 7.8 Hz, 1H), 3.87 (s, 3H), 3.86 (s, 3H), 3.50 (s, 2H); HRMS (ESI-TOF) m/z calcd. for C₃₄H₃₀NO₇⁻ [M+H]⁺ 564.2021, found 564.2017, (Δ=0.66 ppm).

### (±)-α-2,4-di (2-methoxyphenyl)-3-[7-(5-pyrimidine)naphthalen-2-yloxycarbonyl]cyclobutane-1-carboxylic acid 1k

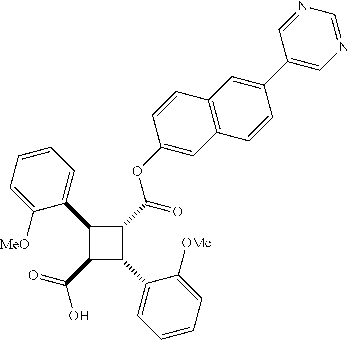 [SMILES: COc1ccccc1[C@@H]1[C@H](C(=O)O)[C@@H](c2ccccc2OC)[C@H]1C(=O)Oc1ccc2cc(-c3cncnc3)ccc2c1]

The same procedure as that for 1g was used, except for purification on C18 silica using acetonitrile/water as eluent. White solid; m.p. 215° C. (decomp.); ¹H NMR (500 MHz, acetone-d₆) δ 10.86 (bs, 1H), 9.19 (s, 2H), 9.18 (s, 1H), 8.31 (s, 1H), 7.91 (m, 3H), 7.62 (d, J=7.3 Hz, 2H), 7.55-7.45 (m, 5H), 7.39 (t, J=7.6 Hz, 2H), 7.29 (t, J=7.3 Hz, 1H), 6.96 (s, 1H), 6.69 (dd, J=8.8, 2.0 Hz, 1H) 4.70 (dd, J=10.6, 7.3, 1H), 4.64 (dd, J=10.6, 7.1, 1H), 4.38 (dd, J=10.7, 7.1, 1H), (4.17 (dd, J=10.6, 7.3 Hz, 1H); ¹³C NMR (125 MHz, DMSO-d₆) δ 172.7, 170.7, 157.3, 154.9, 148.4, 139.1, 132.9, 132.8, 131.0, 129.6, 128.5, 128.3, 128.2, 128.1, 127.8, 127.2, 126.8, 126.1, 125.2, 121.9, 118.2, 46.3, 45.9, 41.6, 40.7. HRMS (ESI-TOF) m/z calculated for C₃₂H₂₄N₂O₄⁺ [M+H]⁺ 501.1809, found=501.1804, (Δ=1.06 ppm).

### (±)-α-3-(2,3-dihydro-1-inden-2-yloxycarbonyl)-2,4-di (2-methoxyphenyl)cyclobutane-1-carboxylic acid 11

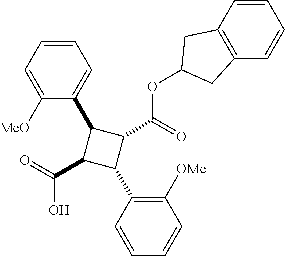

The same procedure as that for 1g was used except for using hexanes/ethyl acetate/acetic acid (75/24/1) as eluent. White solid; m.p. 152-154° C.; ¹H NMR (500 MHz, CD₃CN) δ 8.70 (s, 1H), 7.35-7.24 (m, 4H), 7.23-7.15 (m, 3H), 7.12 (d, J=6.3 Hz, 1H), 7.02-6.94 (m, 3H), 6.82 (d, J=8.1 Hz, 1H), 5.18 (tt, J=6.1, 2.0 Hz, 1H), 4.55 (dd, J=10.7, 6.7 Hz, 1H), 4.51 (dd, J=10.4, 8.0 Hz, 1H), 3.97 (dd, J=10.4, 8.0 Hz, 1H), 3.83 (s, 2H), 3.81 (dd, J=10.6, 6.6 Hz, 2H), 3.52 (s, 3H), 3.12 (dd, J=17.1, 6.3 Hz, 1H), 2.89 (dd, J=17.1, 6.1 Hz, 1H), 2.77 (d, J=17.0 Hz, 1H), 1.88 (d, J=17.0 Hz, 1H). ¹³C NMR (125 MHz, CD₃CN) δ 173.7, 173.2, 158.6, 158.4, 141.8, 129.3, 129.2, 128.5, 128.4, 128.1, 128.0, 127.5, 127.4, 126.0, 125.4, 121.3, 121.1, 111.6, 111.5, 76.0, 56.0, 55.6, 45.9, 45.0, 40.1, 40.0, 37.5, 37.4; HRMS (ESI-TOF) m/z calcd. for C₂₉H₂₉O₆⁺ [M+H]⁺ 473.1959, found: 473.1954 (Δ=0.90 ppm).

### (±)-α-3-[(3,4-dihydrobenzo[b][1,4]dioxin-5-yl)phenoxycarbonyl]-2,4-di (2-methoxyphenyl)-cyclobutanscarboxylic acid 1m

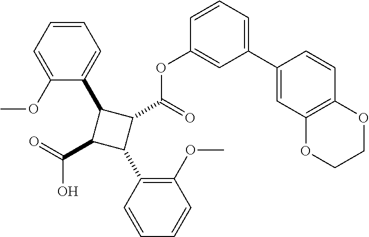

The same procedure as that for 1g was used. White solid; m.p. 169-171° C.; ¹H NMR (700 MHz, acetone-d₆) δ 10.54 (bs, 1H), 7.47 (d, J=7.4 Hz, 2H), 7.42-7.36 (m, 2H), 7.29 (dd, J=8.0 Hz, 1H), 7.27 (m, 1H), 7.08 (m, 2H), 7.03-6.97 (m, 4H), 6.95-6.91 (m, 1H), 6.54 (dd, J=8.0, 1.4 Hz, 1H), 6.42 (t, J=1.8 Hz, 1H), 4.87 (dd, J=10.6, 8.0 Hz, 1H), 4.78 (dd, J=10.5, 6.8 Hz, 1H), 4.34 (q, J=5.0 Hz, 4H), 4.25 (dd, J=10.5, 6.8 Hz, 1H), 4.13 (dd, J=10.5, 8.0 Hz, 1H), 3.89 (s, 3H), 3.88 (s, 3H). ¹³C NMR (175 MHz, acetone-d₆) δ 173.9, 172.4, 159.4, 159.2, 152.8, 145.4, 145.2, 143.0, 134.3, 130.8, 130.0, 129.5, 128.97, 128.89, 128.88, 128.84, 124.7, 121.8, 121.6, 121.4, 121.1, 121.0, 118.9, 116.9, 111.9, 111.8, 65.8, 65.8, 56.5, 56.3, 46.6, 45.6, 38.3, 37.7; HRMS (ESI⁺) m/z calcd. for C₃₄H₃₁O₈⁺ [M+H]⁺ 567.2013, found 567.2023 (A 1.76 ppm).

### (±)-α-3-[2-(bnzo[d][1,3]dioxol-5-yl)phenoxycarbonyl]-2,4-di (2-methoxyphenyl)cyclobutane-1-carboxylic acid 1n

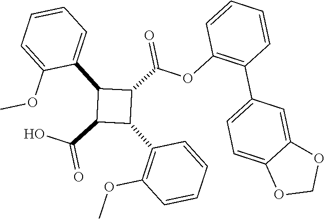

The same procedure as that for 1g was used. White solid; m.p. 139-141° C.; ¹H NMR (700 MHz, acetone-d₆) δ 10.46 (bs, 1H), 7.43 (dd, J=7.5, 1.7 Hz, 1H), 7.35 (td, J=7.8, 1.6 Hz, 1H), 7.32 (dd, J=7.6, 1.7 Hz, 1H), 7.23 (m, 2H), 7.19-7.14 (m, 1H), 7.16-7.11 (m, 2H), 7.08 (d, J=8.2 Hz, 1H), 7.04 (td, J=7.5, 1.1 Hz, 1H), 6.97-6.91 (m, 2H), 6.84-6.79 (m, 3H), 6.05 (dd, J=8.1, 1.3 Hz, 1H), 6.01 (m, 2H), 4.72 (dd, J=10.6, 6.8 Hz, 1H), 4.56 (dd, J=10.5, 7.7 Hz, 1H), 4.09 (dd, J=10.2, 7.8 Hz, 1H), 4.01 (ddd, J=10.5, 6.8, 1.1 Hz, 1H), 3.88 (s, 3H), 3.83 (s, 3H). ¹³C NMR (175 MHz, acetone-d₆) δ 173.7, 171.3, 158.7, 158.5, 148.7, 148.6, 148.0, 135.4, 132.2, 131.2, 129.1, 128.8, 128.8, 128.3, 128.3, 128.2, 128.0, 126.9, 123.6, 123.2, 121.3, 121.0, 111.3, 111.1, 109.9, 108.9, 102.1, 55.9, 55.7, 45.7, 45.5, 37.7, 37.6; HRMS (ESI-TOF) m/z calcd. for C₃₃H₂₉O₈[M+H]⁺ 553.1857, found 553.1868 (A 2.0 ppm).

### (±)-α-3-(1,1′-biphenyl-2-yloxycarbonyl)-2,4-di (2-methoxyphenyl)cyclobutane-1-carboxylic acid 1o

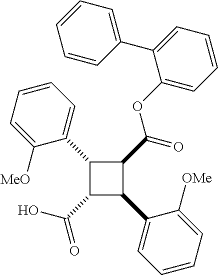 [SMILES: COc1ccccc1[C@H]1[C@H](C(=O)O)[C@H](c2ccccc2OC)[C@H]1C(=O)Oc1ccccc1-c1ccccc1]

The same procedure as that for 1g was used. White solid; m.p.=170-171° C.; ¹H NMR (500 MHz, acetone-d₆) δ 10.43 (bs, 1H), 7.43 (dd, J=7.6, 1.7 Hz, 1H), 7.36 (s, 7H), 7.28-7.21 (m, 2H), 7.19 (dd, J=7.8, 1.4 Hz, 1H), 7.08 (d, J=8.4 Hz, 2H), 7.04 (t, J=7.5 Hz, 1H), 6.95 (d, J=8.2 Hz, 1H), 6.91 (t, J=7.4 Hz, 1H), 6.07 (dd, J=8.0, 1.3 Hz, 1H), 4.71 (dd, J=10.6, 6.8 Hz, 1H), 4.56 (dd, J=10.5, 7.8 Hz, 1H), 4.04 (dd, J=10.6, 7.8 Hz, 1H), 4.00 (dd, J=10.5, 6.8 Hz, 1H), 3.87 (s, 3H), 3.83 (s, 3H); ¹³C NMR (125 MHz, acetone-d₆) δ 173.6, 171.3, 158.7, 158.5, 148.7, 138.4, 135.6, 131.2, 129.6, 129.2, 129.1, 128.8, 128.35, 128.28, 128.24, 128.20, 128.0, 126.9, 123.6, 121.3, 121.0, 111.4, 111.1, 55.9, 55.7, 45.7, 45.5, 37.7, 37.5; HRMS (ESI-TOF) m/z calcd. for C₃₂H₂₉O₆⁺ [M+H]⁺ 509.1959, found 509.1966 (Δ1.4 ppm).

### (±)-3-(4′-cyano-1,1′-biphenyl-3-yloxycarbonyl)-2,4-di (2-methoxyphenyl)cyclobutane-1-carboxylic acid 1p

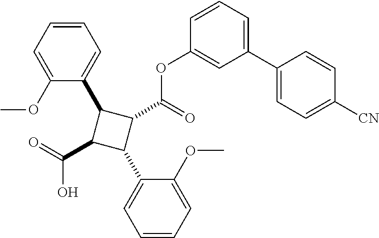

The same procedure as that for 1g was used. White solid; ¹H NMR (500 MHz, acetone-d₆) δ 10.48 (bs, 1H), 7.89 (d, J=8.4 Hz, 2H), 7.73 (d, J=8.4 Hz, 2H), 7.52 (d, J=8.1 Hz, 1H), 7.46 (t, J=7.0 Hz, 2H), 7.40 (d, J=8.1 Hz, 1H), 7.37 (d, J=6.4 Hz, 1H), 7.26 (td, J=7.8, 1.7 Hz, 1H), 7.11-7.02 (m, 2H), 6.98 (t, J=8.6 Hz, 2H), 6.67 (dd, J=8.0, 2.2 Hz, 1H), 6.52 (t, J=2.0 Hz, 1H), 4.87 (dd, J=10.7, 7.8 Hz, 1H), 4.77 (dd, J=10.6, 6.7 Hz, 1H), 4.25 (dd, J=10.7, 6.7 Hz, 1H), 4.12 (dd, J=10.6, 7.8 Hz, 1H), 3.87 (s, 3H), 3.86 (s, 3H); ¹³C NMR (125 MHz, acetone-d₆) δ 172.5, 170.9, 158.1, 157.8, 151.5, 144.0, 140.1, 132.7, 129.9, 128.6, 128.1, 127.8, 127.6, 127.5, 127.45, 127.41, 124.2, 121.9, 120.5, 120.4, 120.2, 118.4, 111.3, 110.5, 110.4, 55.1, 54.9, 45.2, 44.2, 36.9, 36.3; HRMS (ESI-TOF) m/z calcd. for C₃₃H₂₈NO₆⁺ [M+H]⁺ 534.1911, found 534.1914 (Δ=0.6 ppm).

### (±)-α-2,4-bis(2-methoxyphenyl)-3-(naphthalen-1-ylmethoxycarbonyl)cyclobutane-1-carboxylic acid 1q

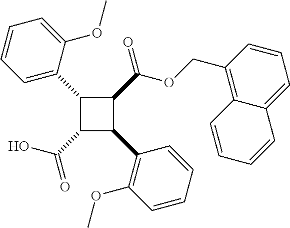 [SMILES: COc1ccccc1[C@H]1[C@H](C(=O)O)[C@H](c2ccccc2OC)[C@H]1C(=O)OCc1cccc2ccccc12]

The same procedure as that for 1g was used. White solid; m.p. 182-184° C.; ¹H NMR (400 MHz, acetone-d₆) δ 10.38 (bs, 1H), 7.95-7.87 (m, 2H), 7.82-7.77 (m, 1H), 7.59-7.51 (m, 2H), 7.46-7.40 (m, 1H), 7.36-7.27 (m, 3H), 7.25-7.18 (m, 2H), 6.99-6.89 (m, 3H), 6.82 (d, J=8.1 Hz, 1H), 5.36 (d, J=12.5 Hz, 1H), 5.12 (d, J=12.5 Hz, 1H), 4.69 (dd, J=10.6, 7.1 Hz, 1H), 4.64 (dd, J=10.3, 7.6 Hz, 1H), 4.07-3.98 (m, 2H), 3.84 (s, 3H), 3.60 (s, 3H). ¹³C NMR (175 MHz, CD₃CN) δ 173.73, 173.23, 158.55, 158.35, 134.59, 132.52, 132.40, 129.95, 129.45, 129.20, 129.19, 128.44, 128.40, 128.31, 128.15, 128.10, 127.52, 126.91, 126.30, 124.59, 121.21, 121.11, 111.44, 111.28, 65.04, 55.94, 55.70, 45.91, 45.32, 37.82, 37.81; HRMS (ESI)⁻ m/z calcd. for C₃₁H₂₇O₆⁻ [M−H]⁺ 495.1813, found 495.1812 (A 0.21 ppm).

### α-2,4-diphenyl-3-(1-naphthaylmethyl)cyclobutane-1-carboxylic acid 1r

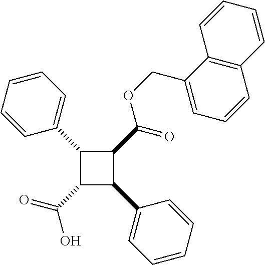 [SMILES: O=C(O)[C@H]1[C@H](c2ccccc2)[C@H](C(=O)OCc2cccc3ccccc23)[C@H]1c1ccccc1]

The same procedure as that for 1g was used. White solid; m.p. 174-176° C.; ¹H NMR (500 MHz, acetone-d₆) δ 4.04 (ddd, J=10.7, 7.2, 3.5 Hz, 2H) 4.43-4.50 (m, 2H) 5.10 (d, J=12.5 Hz, 1H) 5.38 (s, 1H) 7.19-7.40 (m, 9H) 7.42-7.50 (m, 3H) 7.50-7.57 (m, 2H) 7.68-7.73 (m, 1H) 7.87-8.09 (m, 2H); ¹³C NMR (500 MHz, acetone-d₆) δ 41.53, 41.73, 46.29, 46.75, 64.26, 123.66, 125.23, 125.83, 126.48, 126.82, 126.95, 127.56, 127.56, 127.59, 127.80, 128.20, 128.25, 128.48, 129.06, 131.43, 131.69, 133.77, 139.28, 139.35, 171.60, 172.03; HRMS (ESI-TOF) m/z calcd. for C₂₉H₂₄O₄⁺ [M−H]⁺ 436.16746, found 436.16742 (Δ−0.4 ppm).

### (±)-α-3-[2-(2,3-dihydrobenzo[b][1,4]dioxin-6-yl)phenoxycarbonyl]-2,4-bis(2-methoxyphenyl)cyclobutane-1-carboxylic acid 1s

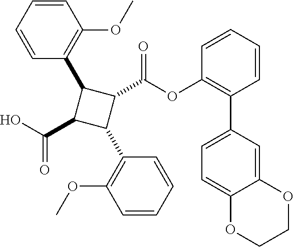

The same procedure as that for 1g was used. White solid; m.p. 139-141° C.; ¹H NMR (700 MHz, acetone-d₆) δ 7.40 (dd, J=7.6, 1.6 Hz, 1H), 7.32 (td, J=7.8, 1.4 Hz, 1H), 7.28 (dd, J=7.6, 1.4 Hz, 1H), 7.22-7.16 (m, 2H), 7.13-7.08 (m, 2H), 7.04 (d, J=8.2 Hz, 1H), 7.00 (td, J=7.5, 1.1 Hz, 1H), 6.93-6.88 (m, 2H), 6.82-6.74 (m, 3H), 5.98 (dd, J=8.1, 1.3 Hz, 1H), 4.69 (dd, J=10.6, 6.7 Hz, 1H), 4.53 (dd, J=10.5, 7.7 Hz, 1H), 4.28-4.19 (m, 4H), 4.05 (dd, J=10.6, 7.7 Hz, 1H), 3.96 (dd, J=10.5, 6.7 Hz, 1H), 3.84 (s, 3H), 3.79 (s, 3H); ¹³C NMR (176 MHz, acetone-d₆) δ 171.4, 158.7, 158.5, 148.7, 144.4, 144.2, 135.1, 131.5, 131.1, 129.1, 128.8, 128.7, 128.5, 128.3, 128.1, 126.8, 123.6, 122.5, 121.3, 121.0, 118.3, 117.8, 111.3, 111.1, 65.2, 65.2, 55.9, 55.7, 45.7, 45.6, 37.7, 37.6; HRMS (ESI-TOF) m/z calcd. for C₃₄H₃₁O₈⁺ [M+H]⁺ 567.2013, found 567.2011 (Δ=0.35 ppm).

### Example 2. Synthesis of γ-Truxillic Acid Monoesters

### (±)-γ-3-((3-(benzo[d][1,3]dioxol-5-yl)phenoxy)carbonyl)-2,4-di(2-methoxyphenyl)cyclobutane-1-carboxylic acid 2a

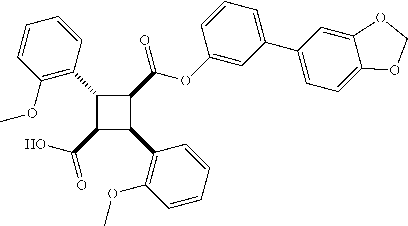 [SMILES: COc1ccccc1[C@H]1[C@H](C(=O)O)[C@H](c2ccccc2OC)[C@@H]1C(=O)Oc1cccc(-c2ccc3c(c2)OCO3)c1]

N,N-Diisopropylethylamine (DIEA) (52.3 μL, 0.3 mmol) was added to a solution of γ-di(2-methoxy)truxillic anhydride (101 mg, 0.3 mmol) in anhydrous THF (0.6 mL), under N₂, followed by the addition of a solution of 3-9benzo-[d][1,3]dioxol-5-yl)phenol (64.3 mg, 0.3 mmol) in anhydrous THF (0.6 mL). The reaction mixture was refluxed overnight. Then the mixture was allowed to cool down to room temperature and water (5 mL) was added. The pH of the solution was adjusted to 3 with 0.1 M solution of HCl. The resulting precipitate was collected by filtration. The crude product collected was purified by column chromatography using hexanes/ethyl acetate as eluant with 20-50% gradient of ethyl acetate. The title compound was isolated (165 mg, 83% yield) as a white solid: m.p. 167-168° C.; ¹H NMR (700 MHz, DMSO-d₆) δ 12.10 (bs, 1H), 7.45-7.25 (m, 6H), 7.09-6.91 (m, 7H), 6.45 (d, J=8.1 Hz, 1H), 6.33 (s, 1H), 6.09 (s, 2H), 4.89 (t, J=10.5 Hz, 1H), 4.61 (t, J=10.4 Hz, 1H), 4.08 (t, J=10.5 Hz, 1H), 3.97 (t, J=10.6 Hz, 1H), 3.82 (s, 3H), 3.75 (s, 3H); ¹³C NMR (125 MHz, acetone-d₆) δ 172.3, 170.1, 158.2, 158.1, 151.4, 148.4, 147.5, 141.8, 134.0, 129.7, 129.4, 129.2, 128.3, 128.1, 126.9, 123.5, 120.5, 120.4, 120.1, 120.08, 119.8, 111.0, 110.8, 108.4, 107.1, 101.4, 55.0, 54.9, 45.3, 44.1, 39.9, 36.1; HRMS (ESI-TOF) m/z calcd. for C₃₃H₂₉O₈[M+H]⁺ 553.1867, found 553.1867 (Δ=0.0 ppm).

### (±)-γ-3-(4-cyano-1,1′-biphenyl-3-yloxycarbonyl)-2,4-diphenylcyclobutane-1-carboxylic acid 2b

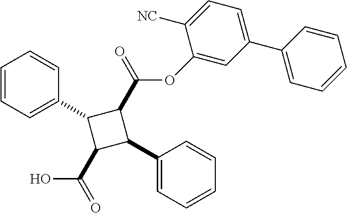

The same procedure as that for 2a was used except for using hexanes/ethyl acetate/acetic acid (75/24/1) as eluant. White solid; m.p. 203-204° C.; ¹H NMR (500 MHz, acetone-d₆) δ 10.89 (s, 1H), 7.84 (d, J=8.1 Hz, 1H), 7.68 (dd, J=8.1, 1.6 Hz, 1H), 7.62-7.19 (m, 15H), 6.09 (d, J=1.5 Hz, 1H), 4.82 (t, J=10.7 Hz, 1H), 4.60 (t, J=10.1 Hz, 1H), 4.30 (t, J=10.3 Hz, 1H), 4.03 (t, J=10.4 Hz, 1H). ¹³C NMR (126 MHz, acetone-d₆) δ 173.42, 171.17, 153.76, 148.25, 140.45, 140.29, 139.13, 134.93, 134.87, 130.48, 130.06, 129.70, 129.58, 129.03, 128.90, 128.56, 128.39, 126.22, 122.33, 116.17, 106.80, 47.95, 47.27, 43.38, 42.59; HRMS (ESI-TOF) m/z calcd. for C₃₁H₂₃NO₄⁺ [M+H]⁺ 474.1700, found=474.1701 (Δ=0.3 ppm).

### (±)-γ-3-((3-(2,3-dihydrobenzo[b][1, 4]dioxin-5-yl) phenoxy) carbonyl)-2,4-di(2-methoxyphenyl)cyclobutane-1-carboxylic acid 2c

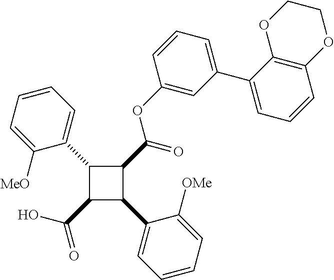 [SMILES: COc1ccccc1[C@H]1[C@H](C(=O)O)[C@H](c2ccccc2OC)[C@@H]1C(=O)Oc1cccc(-c2cccc3c2OCCO3)c1]

The same procedure as that for 2a was used except for using hexanes/ethyl acetate/acetic acid (75/24/1) as eluant. White solid; m.p. 194-195° C.; ¹H NMR (500 MHz, acetone-d₆) δ 10.53 (s, 1H), 7.53 (dd, J=7.5, 0.8 Hz, 1H), 7.37 (d, J=7.4 Hz, 1H), 7.34-7.20 (m, 4H), 7.03 (t, J=8.6 Hz, 2H), 6.99-6.88 (m, 3H), 6.85 (dd, J=8.0, 1.6 Hz, 1H), 6.78 (dd, J=7.5, 1.6 Hz, 1H), 6.64-6.56 (m, 1H), 6.47 (dd, J=8.0, 1.2 Hz, 1H), 5.02 (t, J=10.6 Hz, 1H), 4.79 (t, J=10.4 Hz, 1H), 4.31 (m, 2H), 4.26 (m, 2H), 4.20 (t, J=10.4 Hz, 1H), 4.13 (t, J=10.6 Hz, 1H), 3.93 (s, 3H), 3.82 (s, 3H); ¹³C NMR (126 MHz, acetone-d₆) δ 170.02, 158.18, 158.04, 150.62, 144.17, 140.78, 138.98, 129.75, 129.56, 129.17, 128.36, 128.14, 128.07, 126.82, 126.39, 122.50, 122.41, 120.80, 120.41, 120.17, 120.04, 116.71, 111.03, 110.79, 64.24, 64.05, 55.01, 54.85, 45.30, 44.16, 39.92; HRMS (ESI-TOF) m/z calcd. for C₃₄H₃₁O₈⁺ [M+H]⁺ 567.2013, found 567.1997 (Δ=2.82).

### (±)-γ-3-(4′-cyano-1,1′-biphenyl]-2-yloxycarbonyl)-2,4-diphenylcyclobutane-1-carboxylic acid 2d

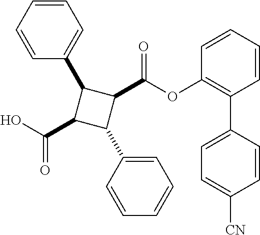

The same procedure as that for 2a was used except for recrystallization from ethyl acetate after flash chromatography. White solid; 77% yield; m.p. 224-225° C.; ¹H NMR (500 MHz, acetone-d₆) δ 10.82 (bs, 1H), 7.76 (d, J=8.4 Hz, 2H), 7.56 (d, J=8.3 Hz, 2H), 7.44-7.21 (m, 11H), 7.15 (d, J=7.5 Hz, 2H), 6.09 (dd, J=8.0, 1.3 Hz, 1H), 4.65 (t, J=10.7 Hz, 1H), 4.43 (t, J=10.1 Hz, 1H), 3.88 (t, J=10.4 Hz, 1H), 3.83 (t, J=10.4 Hz, 1H); ¹³C NMR (175 MHz, acetone-d₆) δ 172.3, 169.4, 147.6, 142.6, 142.2, 138.2, 134.0, 132.1, 130.1, 129.7, 129.5, 128.8, 128.4, 128.3, 127.7, 127.2, 126.6, 126.5, 126.2, 122.6, 118.6, 46.1, 45.9, 44.4, 41.5; HRMS (ESI-TOF) m/z calcd. for C₃₁H₂₄NO₄⁺ [M+H]⁺ 474.1700, found 474.1703 (Δ=0.63 ppm).

### (±)-γ-3-(4′-fluoro-1,1′-biphenyl-3-yl)oxycarbonyl-2,4-di (2-methoxyphenyl)cyclobutane-1-carboxylic acid 2e

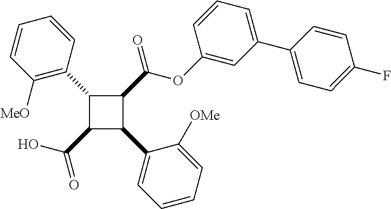 [SMILES: COc1ccccc1[C@H]1[C@H](C(=O)O)[C@H](c2ccccc2OC)[C@@H]1C(=O)Oc1cccc(-c2ccc(F)cc2)c1]

The same procedure as that for 2a was used. White solid; m.p. 187-188° C.; ¹H NMR (500 MHz, CDCl₃) δ 7.43-7.37 (m, 3H), 7.33 (d, J=7.2 Hz, 1H), 7.28-7.23 (m, 4H), 7.10 (t, J=8.6 Hz, 2H), 6.98-6.91 (m, 3H), 6.84 (d, J=8.2 Hz, 1H), 6.44 (d, J=7.6 Hz, 1H), 6.42 (s, 1H), 4.85 (t, J=10.8 Hz, 1H), 4.76 (t, J=10.0 Hz, 1H), 4.24 (t, J=10.4 Hz, 1H), 4.12 (t, J=10.4 Hz, 1H), 3.88 (s, 3H), 3.70 (s, 3H); ¹³C NMR (125 MHz, CDCl₃) δ 177.8, 170.5, 162.6 (d, J_{CR}=246.8 Hz), 158.0, 157.8, 150.9, 141.4, 136.2 (d, J=3.3 Hz), 130.0, 129.6, 129.5, 129.1, 128.71 (d, J=8.0 Hz), 128.5, 128.4, 126.1, 124.0, 120.8, 120.6, 120.2, 120.0, 115.5 (d, J=21.4 Hz), 110.8, 110.7, 55.2, 55.0, 44.9, 44.2, 40.5, 38.4; HRMS (ESI-TOF) m/z calcd. for C₃₁H₂₆NO₆⁺ [M+H]⁺ 527.1864, found 527.1871 (Δ=1.3 ppm).

### (±)-γ-2,4-di (2-methoxyphenyl)-3-(naphthalen-1-yloxycarbonyl)cyclobutane-1-carboxylic acid 2f

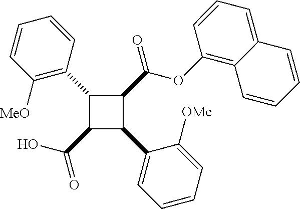 [SMILES: COc1ccccc1[C@H]1[C@H](C(=O)O)[C@H](c2ccccc2OC)[C@@H]1C(=O)Oc1cccc2ccccc12]

The same procedure as that for 2a was used. White solid; m.p. 179-180° C.; ¹H NMR (500 MHz, CDCl₃) δ 7.76 (d, J=8.1 Hz, 1H), 7.61 (d, J=8.2 Hz, 1H), 7.49 (d, J=7.4 Hz, 1H), 7.41-7.21 (m, 7H), 7.01-6.90 (m, 4H), 6.45 (d, J=7.5 Hz, 1H), 4.95 (t, J=10.8 Hz, 1H), 4.88 (t, J=10.1 Hz, 1H), 4.38 (t, J=10.4 Hz, 1H), 4.15 (t, J=10.5 Hz, 1H), 3.89 (s, 3H), 3.74 (s, 3H); ¹³C NMR (125 MHz, CDCl₃) δ 178.0, 170.3, 158.0, 157.8, 146.6, 134.4, 130.1, 129.4, 129.2, 128.7, 128.3, 127.6, 126.8, 126.1, 126.0, 125.9, 125.5, 125.3, 121.4, 120.8, 117.6, 110.8, 55.2, 55.0, 45.4, 44.3, 40.4, 38.3; HRMS (ESI-TOF) m/z calcd. for C₃₀H₂₇O₆⁺ [M+H]⁺ 483.1802, found 483.1794 (Δ=1.65 ppm).

### (±)-γ-3-(4′-cyano-1,1′-biphenyl]-3-yloxycarbonyl)-2,4-di (2-methoxyphenyl)cyclobutane-1-carboxylic acid 2g

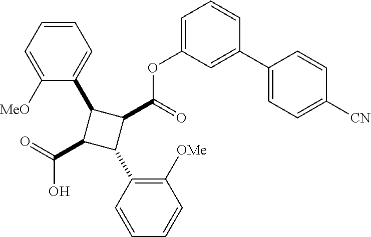

The same procedure as that for 2a was used. White solid; m.p. 203-204° C.; ¹H NMR (500 MHz, acetone-d₆) δ 7.90 (s, 2H), 7.74 (s, 2H), 7.54 (d, J=21.7 Hz, 2H), 7.45-7.17 (m, 4H), 7.01 (d, J=40.4 Hz, 4H), 6.68 (s, 1H), 6.52 (s, 1H), 5.05 (s, 1H), 4.80 (s, 1H), 4.19 (d, J=39.6 Hz, 2H), 3.87 (d, J=55.4 Hz, 6H). ¹³C NMR (126 MHz, acetone-d₆) δ 171.50, 159.58, 152.98, 145.50, 141.51, 134.08, 131.28, 130.62, 129.71, 129.50, 129.18, 125.51, 123.34, 121.83, 121.53, 112.45, 112.26, 56.47, 56.28, 46.72, 41.30; HRMS (ESI-TOF) m/z calcd. for C₃₃H₂₇NO₆ [M+H]⁺ 534.1911, found 534.1914 (Δ=0.56 ppm).

### (±)-γ-3-(4′-cyano-1,1′-biphenyl]-2-yloxycarbonyl)-2,4-di (2-methoxyphenyl)cyclobutane-1-carboxylic acid 2h

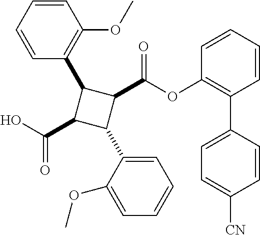

The same procedure as that for 2a was used. White solid; m.p. 168-169° C.; ¹H NMR (700 MHz, acetone-d₆) δ 10.48 (s, 1H), 7.66 (d, J=8.3 Hz, 2H), 7.53 (d, J=8.3 Hz, 2H), 7.47 (d, J=5.4 Hz, 1H), 7.43 (dd, J=7.3, 2.0 Hz, 1H), 7.33-7.25 (m, 4H), 7.12 (d, J=7.4 Hz, 1H), 7.01 (dd, J=16.4, 8.5 Hz, 2H), 6.93 (dt, J=11.2, 7.5 Hz, 2H), 6.23 (d, J=7.9 Hz, 1H), 4.93 (t, J=10.7 Hz, 1H), 4.71 (t, J=10.4 Hz, 1H), 3.99 (td, J=10.5, 4.9 Hz, 1H), 3.82 (d, J=2.2 Hz, 3H), 3.74 (d, J=2.4 Hz, 3H). ¹³C NMR (126 MHz, acetone-d₆) δ 171.88, 169.61, 158.05, 158.02, 147.81, 142.14, 132.83, 131.99, 130.18, 129.76, 129.50, 129.36, 129.11, 128.79, 128.17, 128.12, 126.55, 126.23, 123.00, 120.39, 120.02, 118.39, 111.07, 111.01, 110.87, 55.02, 54.68, 44.95, 44.50, 39.56, 36.24. HRMS (ESI-TOF) m/z calculated for C₃₁H₂₄NO₄⁺ [M+H]⁺ 474.1700, found 474.1696 (Δ=−0.8 ppm).

### (±)-γ-3-[3-(2-oxoindolin-6-yl)phenoxycarbonyl)-2,4-diphenylcyclobutane-1-carboxylic acid 2i

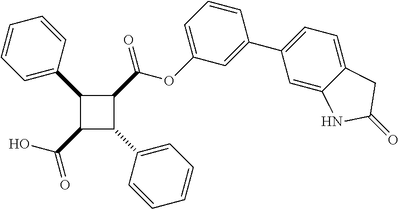 [SMILES: O=C1Cc2ccc(-c3cccc(OC(=O)[C@H]4[C@H](c5ccccc5)[C@@H](C(=O)O)[C@H]4c4ccccc4)c3)cc2N1]

A procedure similar to that for 2a was used. Instead of column chromatography on silica gel, the precipitate from the reaction mixture was collected by filtration and purified by recrystallization from methanol/water to afford the title compound. White solid; m.p.>230° C.; ¹H NMR (700 MHz, DMSO-d₆) δ 12.29 (bs, 1H), 10.59 (s, 1H), 7.47-7.25 (m, 13H), 7.04 (d, J=7.8 Hz, 1H), 6.82 (s, 1H), 6.53 (d, J=8.3 Hz, 1H), 6.16 (s, 1H), 4.54 (t, J=10.6 Hz, 1H), 4.39 (t, J=10.1 Hz, 1H), 4.14 (t, J=10.4 Hz, 1H), 3.85 (t, J=10.5 Hz, 1H), 3.53 (s, 2H); ¹³C NMR (175 MHz, DMSO-d₆) δ 177.1, 173.0, 170.8, 150.9, 144.9, 142.4, 142.1, 138.91, 138.87, 130.3, 129.1, 128.94, 128.91, 127.9, 127.21, 127.16, 126.1, 125.3, 124.4, 120.9, 120.3, 119.9, 107.7, 46.2, 46.1, 44.5, 42.3, 36.1; HRMS (ESI-TOF) m/z calcd. for C₃₂H₂₆O₅⁺ [M+H]⁺ 504.1805, found 504.1804, (Δ=0.20 ppm).

### (±)-γ-3-[3-(2-oxo-1,2,3,4-tetrahydroquinolin-6-yl)phenoxycarbonyl)-2,4-diphenylcyclobutane-1-carboxylic acid 2j

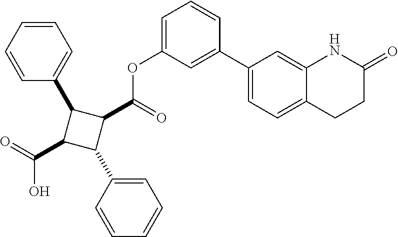

The same procedure as that for 2a was used except for using methanol/dichloromethane (2-10% gradient). Gray solid; m.p. 230° C. (decomp.); ¹H NMR (500 MHz, DMSO-d₆): δ 12.28 (bs, 1H), 10.16 (s, 1H), 7.62-7.16 (m, 13H), 6.98 (d, J=8.0 Hz, 1H), 6.97 (s, 1H), 6.44 (m, 1H), 6.30 (s, 1H), 4.55 (t, J=10.7 Hz, 1H), 4.39 (t, J=10.1 Hz, 1H), 4.14 (t, J=10.3 Hz, 1H), 3.84 (t, J=10.4 Hz, 1H), 2.92 (t, J=7.4 Hz, 2H), 2.48 (ovlp with DMSO, 2H, redo in acetone); ¹³C NMR (125 MHz, DMSO-d₆) δ 173.0, 170.7 (two overlapping carbons), 151.0, 142.3, 141.8, 139.3, 138.9, 138.4, 130.3, 129.0, 128.9, 128.8, 127.7, 127.21, 127.17, 124.3, 123.8, 120.8, 119.8, 113.6, 46.3, 46.1, 44.5, 42.3, 30.8, 25.0; HRMS (ESI-TOF) m/z calcd for C₃₃H₂₈NO₅ [M+H]⁺ 518.1962, found 518.1966 (Δ=0.77 ppm).

### (±)-γ-3-[3-(2,3-dihydrobenzo[b][1,4]dioxin-6-yl)phenoxycarbonyl]-2,4-di(2-methoxyphenyl)cyclobutane-1-carboxylic acid 2k

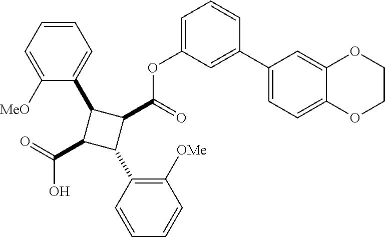

The same procedure as that for 2a was used. ¹H NMR (300 MHz, acetone-d₆) δ 7.55 (d, J=7.2 Hz, 1H), 7.45-7.16 (m, 5H), 7.16-6.81 (m, 7H), 6.52 (d, J=7.3 Hz, 1H), 6.39 (s, 1H), 5.03 (t, J=10.5 Hz, 1H), 4.78 (t, J=10.2 Hz, 1H), 4.30 (s, 4H), 4.17 (dt, J=20.8, 10.5 Hz, 2H), 3.85 (d, J=32.0 Hz, 6H); ¹³C NMR (176 MHz, Acetone) 6 39.91, 44.13, 45.28, 54.84, 55.00, 64.04, 64.24, 110.78, 111.02, 116.71, 120.04, 120.17, 120.41, 120.80, 122.41, 122.49, 126.40, 126.80, 128.08, 128.14, 128.37, 129.10, 129.17, 129.55, 129.72, 138.97, 140.77, 144.16, 150.61, 158.03, 158.17, 170.03, 172.36; HRMS (ESI-TOF) m/z calcd. for C₃₄H₃₁O₈⁺ [M+H]⁺ 567.2013, found 567.2023 (Δ=1.8 ppm).

### (±)-γ-3-(2,3-dihydro-1H-inden-2-yloxycarbonyl)-2,4-bis(2-methoxyphenyl)cyclobutane-1-carboxylic acid 21

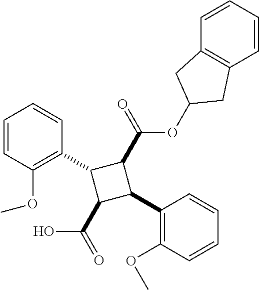

The same procedure as that for 2a was used except for the second column using 2.5% methanol in dichloromethane. White solid; m.p. 185-186° C.; 1H NMR (700 MHz, DMSO-d₆) δ 11.98 (bs, 1H), 7.31-7.10 (m, 7H), 7.07-7.04 (m, 1H), 6.97 (d, J=8.0 Hz, 1H), 6.93-6.87 (m, 2H), 6.86 (d, J=8.2 Hz, 1H), 5.07 (tt, J=6.2, 2.1 Hz, 1H), 4.63 (t, J=10.5 Hz, 1H), 4.50 (t, J=10.5 Hz, 1H), 3.82 (t, J=10.7 Hz, 1H), 3.76 (s, 3H), 3.70 (t, J=10.4 Hz, 1H), 3.47 (s, 3H), 3.06 (dd, J=17.0, 6.2 Hz, 1H), 2.81 (dd, J=17.0, 6.0 Hz, 1H), 2.68 (d, J=16.8 Hz, 1H), 1.74 (d, J=16.8 Hz, 1H); ¹³C NMR (175 MHz, DMSO-d₆) δ 172.9, 170.7, 157.5, 157.1, 140.4, 140.3, 129.5, 128.7, 128.1, 127.9, 127.8, 126.3, 126.2, 126.1, 124.6, 124.3, 120.3, 119.6, 111.2, 110.9, 74.2, 55.3, 54.8, 44.9, 43.9, 40.0, 38.75, 38.70, 38.5; HRMS (ESI-TOF) m/z calcd. for C₂₉H₂₈O₆[M+H]⁺ 473.1963, found 473.1963, (Δ=0.0 ppm).

### (±)-γ-2,4-diphenyl-3-[6-(pyrimidin-5-yl)naphthalen-2-yloxy)carbonyl]cyclobutane-1-carboxylic acid 2m

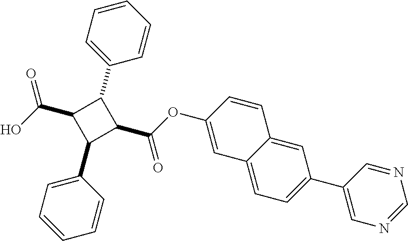 [SMILES: O=C(O)[C@H]1[C@H](c2ccccc2)[C@@H](C(=O)Oc2ccc3cc(-c4cncnc4)ccc3c2)[C@H]1c1ccccc1]

Disopropylethylamine was added dropwise to a DMF (1.0 mL, 0.21 M) solution of γ-truxillic anhydride (62 mg, 0.22 mmol) and 6-(pyrimidin-5-yl)naphthalen-2-ol (50 mg, 0.21 mmol). The resulting yellowish suspension was heated to 100° C. (becomes a solution at temperatures higher than 60° C.) for 3 hours and at 60° C. for 12 hours. Then, the reaction mixture was filtered, the collected solid was washed with water (1 mL) and ethyl acetate (1 mL) to give the title compound (25 mg) as a colorless solid. The mother liquor was diluted with water (2.0 mL) and cooled to 0° C. in ice/water, then 5% aqueous solution of NaH₂PO₄ was added dropwise until pH reached 5. The aqueous layer was extracted with ethyl acetate and a precipitate formed. The suspension was filtered on a Büchner's funnel to collect the solid, which was air-dried overnight to afford the additional title compound (54 mg). Thus the combined total yield (79 mg) of the title compound was 75%. ¹H NMR (500 MHz, DMSO-d₆) δ 12.30 (bs, 1H), 9.26 (s, 2H), 9.22 (s, 1H), 8.47-8.29 (m, 1H), 7.96 (dd, J=8.5, 1.7 Hz, 1H), 7.87 (t, J=7.9 Hz, 2H), 7.48-7.36 (m, 9H), 7.29 (t, J=7.2 Hz, 1H), 6.90 (d, J=2.3 Hz, 1H), 6.53 (dd, J=8.9, 2.3 Hz, 1H), 4.58 (t, J=10.7 Hz, 1H), 4.43 (t, J=10.1 Hz, 1H), 4.20 (t, J=10.4 Hz, 1H), 3.87 (t, J=10.5 Hz, 1H); ¹³C NMR (125 MHz, DMSO-d₆) δ 172.9, 170.8, 157.8, 155.4, 148.8, 142.3, 138.9, 133.4, 133.3, 131.5, 130.1, 129.1, 129.0, 128.9, 128.8, 127.7, 127.2, 126.5, 125.6, 125.2, 122.3, 118.6, 46.3, 46.2, 44.6, 42.3; HRMS (ESI-TOF) m/z calcd. C₃₂H₂₅N₂O₄⁺ [M+H]⁺ 501.18088, found 501.18181 (Δ=1.84 ppm)

### (±)-γ-3-[(3-(2-oxo-1,2,3,4-tetrahydroquinolin-6-yl)phenoxycarbonyl]-2,4-diphenylcyclobutane-1-carboxylic acid 2n

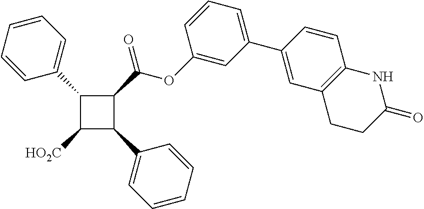 [SMILES: O=C1CCc2cc(-c3cccc(OC(=O)[C@H]4[C@@H](c5ccccc5)[C@@H](C(=O)O)[C@@H]4c4ccccc4)c3)ccc2N1]

To N₂ protected γ-(2-MeO)-truxillic anhydride (220 mg, 0.79 mmol) was added dry THF (6.0 mL) was then added followed by DIPEA (69 μL, 0.395 mmol) and 6-(3-hydroxyphenyl)-3,4-dihydroquinolin-2 (1H)-one (95 mg, 0.395 mmol). The resulting suspension was then heated to reflux at 65° C. and stirred for 34 h. The reaction was monitored by TLC (50% ethyl acetate/hexane) to completion. The white precipitate was then isolated by filtration with THF. The crude was recrystallized from EtOH to afford the title compound (147 mg, 72% yield) as white solid: m.p.>230° C.; ¹H NMR (700 MHz, acetone-d₆) δ 10.77 (s, 1H), 9.17 (s, 1H), 7.52 (dd, J=24.0, 7.6 Hz, 4H), 7.46-7.25 (m, 10H), 7.04 (d, J=8.5 Hz, 1H), 6.49 (d, J=7.8 Hz, 1H), 4.78 (t, J=11.0 Hz, 1H), 4.53 (t, J=10.2 Hz, 1H), 4.17 (t, J=10.5 Hz, 1H), 3.98 (t, J=10.5 Hz, 1H), 3.08 (t, J=7.3 Hz, 2H), 2.58 (t, J=7.4 Hz, 2H); ¹³C NMR (176 MHz, acetone-d₆) δ 173.0, 170.7, 170.7, 151.0, 142.3, 141.6, 138.9, 138.7, 132.8, 130.2, 129.1, 128.9, 128.9, 127.7, 127.2, 127.2, 126.4, 125.8, 124.6, 123.9, 120.3, 119.3, 115.9, 46.3, 46.1, 44.6, 42.3, 30.8, 25.3; HRMS (ESI-TOF) m/z calcd. for C₂₉H₂₈O₆ [M+H]⁺ 518.1962, found 518.1966, (Δ=0.83 ppm).

### Example 3. Synthesis of ε-Truxillic Acid Monoesters

(±)-ε-3-[(2,3-dihydro-1H-inden-2-yl)oxycarbonyl]-2,4-diphenylcyclobutane-1-carboxylic acid 3a

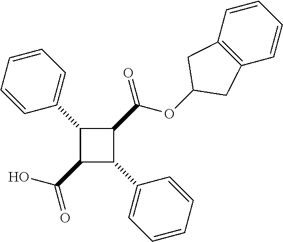 [SMILES: O=C(O)[C@@H]1[C@@H](c2ccccc2)[C@H](C(=O)OC2Cc3ccccc3C2)[C@H]1c1ccccc1]

To a solution of ε-truxillic acid (100 mg, 0.359 mmol) in anhydrous dichloromethane (5.00 mL) under nitrogen atmosphere was added N-(3-dimethylaminopropyl)-N′-ethylcarbodiimide hydrochloride (EDC·HCl) (76.0 mg, 0.400 mmol), dimethylaminopyridine (DMAP) (49 mg, 0.337 mmol). The mixture was stirred for 30 min and 2-indanol (54.0 mg, 0.402 mmol) was added. The mixture was stirred at room temperature for 15 hours, and the reaction mixture was diluted with dichloromethane (20 mL) and water (10 mL). The pH of the reaction mixture was adjusted to 4-5 by addition of 5% NaH₂PO₄. The aqueous layer was extracted with dichloromethane (20 mL×3), and the combined organic layers were washed with brine and dried over anhydrous magnesium sulfate. The solvent was removed under reduced pressure and the resulting crude material was purified by flash chromatography on silica gel using hexanes/ethyl acetate (3/1->1.1) as eluent to give the title compound (40 mg, 27% yield, not optimized) as a colorless solid: m.p. 147-148° C.; ¹H NMR (500 MHz, acetone-d₆) δ 11.07 (bs, 1H), 7.38 (dd, J=7.5, 1.7 Hz, 4H), 7.33-7.13 (m, 10H), 5.62 (tt, J=6.1, 2.7 Hz, 1H), 3.90 (t, J=9.7 Hz, 2H), 3.40 (t, J=9.7 Hz, 1H), 3.36 (dd, J=16.9, 6.1 Hz, 2H), 3.20 (t, J=9.7 Hz, 1H), 3.01 (dd, J=16.9, 2.7 Hz, 2H). ¹³C NMR (125 MHz, acetone-d₆) δ 173.2, 172.2, 141.1, 140.5, 128.4, 126.8, 127.7, 126.6, 124.5, 75.9, 49.0, 47.1, 43.1, 39.3; HRMS (ESI-TOF) m/z calcd. for C₂₇H₂504′[M+H]⁺ 413.1747, found 413.1751 (Δ=1.0 ppm).

### (±) -ε-3-(1,1′-biphenyl-3-yloxycarbonyl)-2,4-diphenylcyclobutane-1-carboxylic acid 3b

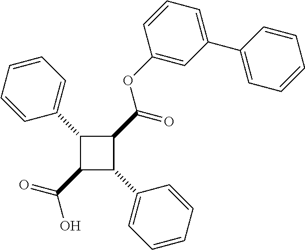 [SMILES: O=C(O)[C@@H]1[C@@H](c2ccccc2)[C@H](C(=O)Oc2cccc(-c3ccccc3)c2)[C@H]1c1ccccc1]

The procedure for 3a was used. White solid; m.p. 149-150° C.; ¹H NMR (500 MHz, acetone-d₆) δ 11.14 (s, 1H), 7.67 (d, J=7.1 Hz, 2H), 7.59 (d, J=7.2 Hz, 5H), 7.50 (dt, J=19.3, 7.9 Hz, 4H), 7.45-7.37 (m, 5H), 7.31 (t, J=7.4 Hz, 2H), 7.20 (ddd, J=7.9, 2.3, 1.1 Hz, 1H), 4.14 (t, J=9.8 Hz, 2H), 3.68 (t, J=9.7 Hz, 1H), 3.50 (t, J=9.7 Hz, 1H). ¹³C NMR (126 MHz, acetone-d₆) δ 173.16, 171.17, 151.47, 142.53, 141.07, 139.87, 129.83, 128.90, 128.58, 127.74, 127.05, 127.01, 126.92, 124.32, 120.63, 120.23, 48.63, 48.05, 43.29. HRMS (ESI-TOF) calculated for C₂₉H₂₄O₄⁺ [M+H]⁺ 449.1747, Found 449.1757 (Δ=−2.26 ppm).

### (±) -ε-3-(1,1′-biphenyl-2-ylmethoxycarbonyl)-2,4-diphenylcyclobutane-1-carboxylic acid 3c

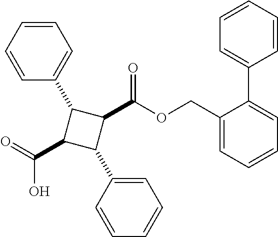

The procedure for 3a was used. White solid; m.p. 121-122° C.; ¹H NMR (500 MHz, acetone-d₆) δ 11.07 (bs, 1H), 7.51 (d, J=7.1 Hz, 1H), 7.48-7.34 (m, 14H), 7.33-7.25 (m, 5H), 5.12 (s, 2H), 3.90 (t, J=9.8 Hz, 2H), 3.41 (t, J=9.8 Hz, 1H), 3.34 (s, 1H); ¹³C NMR (101 MHz, acetone-d₆) δ 142.78, 131.31, 130.19, 128.63, 128.59, 125.67, 122.24, 121.77, 121.57, 110.10, 108.86, 103.02, 50.35, 45.02; HRMS (ESI-TOF) m/z calcd. for C₃₁H₂₇O₄⁺ [M+H]⁺ 463.1904, found 463.1921 (Δ=3.64 ppm)

### (±)-ε-3-[3-(2,3-dihydrobenzo[b][1,4]dioxin-6-yl)phenoxycarbonyl]-2,4-diphenylcyclobutane-1-carboxylic acid 3d

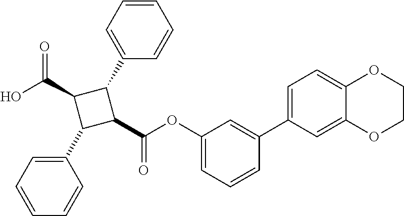 [SMILES: O=C(O)[C@@H]1[C@@H](c2ccccc2)[C@H](C(=O)Oc2cccc(-c3ccc4c(c3)OCCO4)c2)[C@H]1c1ccccc1]

The procedure for 3a was used. White solid; ¹H NMR (500 MHz, acetone-d₆) δ 7.57 (dd, J=7.5, 1.7 Hz, 4H), 7.50-7.34 (m, 7H), 7.33-7.21 (m, 2H), 7.15-7.05 (m, 3H), 6.94-6.84 (m, 1H), 4.36-4.24 (m, 4H), 4.11 (t, J=9.7 Hz, 2H), 3.64 (t, J=9.7 Hz, 1H), 3.48 (t, J=9.7 Hz, 1H); ¹³C NMR (126 MHz, acetone-d₆) δ 172.1, 152.3, 145.0, 144.7, 142.9, 142.0, 134.0, 130.6, 129.5, 127.9, 127.9, 124.7, 121.0, 120.7, 120.6, 118.4, 116.4, 65.3, 65.2, 49.56, 49.0, 44.2; HRMS (ESI-TOF) m/z calcd. for C₃₂H₂₇O₆⁺ [M+H]⁺ 507.1802, found 507.1814 (Δ=2.4 ppm).

### ε-2,4-diphenyl-3-(4-fluorophenyl)phenoxycarbonylcyclobutane-1-carboxylic acid 3e

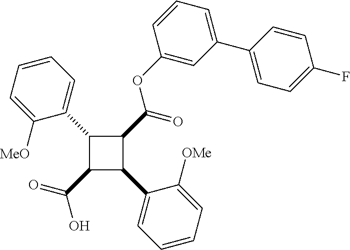 [SMILES: COc1ccccc1[C@H]1[C@H](C(=O)O)[C@H](c2ccccc2OC)[C@@H]1C(=O)Oc1cccc(-c2ccc(F)cc2)c1]

The procedure for 3a was used. White solid; 153-154° C.; ¹H NMR (700 MHz, acetone-d₆) δ 3.5 (t, J=9.7, 9.7, 1H), 3.67 (t, J=9.9, 9.9, 1H), 4.13 (t, J=9.9, 9.9, 2H), 7.20 (dd, J=7.7, 1.3, 1H), 7.24 (m, 2H), 7.31 (t, J=7.5, 7.5, 2H), 7.42 (t, J=7.5, 7.5, 4H) 7.5 (m, 2H), 7.56 (m, 5H), 7.72 (m, 2H); ¹³C NMR (176 MHz, acetone-d₆) δ 43.3, 48.1, 48.6, 115.5, 115.6, 120.2, 124.2, 127.0, 128.6, 128.9, 129.9, 136.2, 141.0, 141.4, 151.4, 163.3, 171.1, 173.2; HRMS (ESI-TOF) calculated for C₃₀H₂₃FO₄⁺ [M+H]⁺ 467.1653, found 467.1653 (Δ=0.0 ppm).

### ε-2,4-diphenyl-3-(napthylen1-yl)methoxycarbonylcyclobutane-1-carboxylic acid 3f

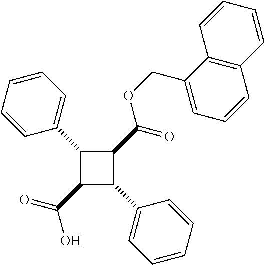 [SMILES: O=C(O)[C@@H]1[C@@H](c2ccccc2)[C@H](C(=O)OCc2cccc3ccccc23)[C@H]1c1ccccc1]

The same procedure as for 3a was used except for the use of dichloromethane as the solvent for the reaction. White solid; m.p. 156-1580 C; ¹H NMR (500 MHz, CD₃CN) 6 3.31 (td, J=9.77, 5.80 Hz, 2H) 3.85 (t, J=9.77 Hz, 2H) 5.64 (s, 2H) 7.23-7.26 (m, 1H) 7.26-7.38 (m, 8H) 7.45-7.51 (m, 1H) 7.51-7.59 (m, 3H) 7.89-7.98 (m, 2H) 8.00-8.06 (m, 1H); ¹³C NMR (500 MHz, CD₃CN) δ 173.12, 172.29, 140.71, 133.71, 131.65, 131.46, 129.22, 128.56, 128.50, 127.64, 127.09, 126.98, 126.62, 126.07, 125.33, 123.83, 117.33, 64.86, 48.71, 47.59, 43.20; HRMS (ESI-TOF) calculated for C₂₉H₂₄O₄⁺ [M+H]⁺ 436.1683, found 436.1683 (Δ=1.48 ppm).

### Example 4. FABP3, FABP5, and FABP7 Binding Assays

Purified FABPs (3 μM) were incubated with fluorescent probe (500 nM) in 30 mM Tris, 100 mM NaCl buffer (pH 7.5). Compounds to be tested were then added to the wells (0.01-50 μM) and the system was allowed to reach equilibrium by incubating in the dark at room temperature for 20 minutes. Each independent assay included wells containing a strong competitive ligand (arachidonic acid, 10 μM) as a positive control for probe displacement. Loss of fluorescence intensity was monitored with a F5 Filtermax Multi-Mode Microplate Reader (Molecular Devices, Sunnyvale, CA, USA) using excitation and emission wavelengths appropriate for each respective probe (NBD-stearate ex./em.=465/535 nm, DAUDA ex./em.=345/535 nm, ANS ex./em.=370/465 nm). Following background subtraction, the fluorescence intensity values were normalized and fit to a one-site binding analysis using the GraphPad Prism software (Prism version 7.0 for Mac OS, Graphpad Software Inc., La Jolla, CA, USA) to determine the K, of the tested compounds from the equation K, =IC₅₀/(1+([Probe]/K_{d}).

TABLE 2
In vitro affinities (Kᵢ) of FABP inhibitors
(1: α-truxillic acid mono ester; 2: γ-truxillic acid mono ester;
3: ε-truxillic acid mono ester)

| | | | | FABP3 Ki/ |
|---|---|---|---|---|
| Compound | FABP3 Kᵢ (μM) | FABP5 Kᵢ (μM) | FABP7 Kᵢ (μM) | FABP5 Ki |
| 1a | 1.06 ± 0.09 | 0.32 ± 0.08 | 0.63 ± 0.09 | 3.3 |
| 1b | 1.60 ± 0.23 | 0.33 ± 0.06 | 0.96 ± 0.12 | 4.8 |
| 1c | 6.00 ± 1.07 | 0.36 ± 0.05 | 0.57 ± 0.09 | 16.7 |
| 1d | 3.37 ± 0.26 | 0.72 ± 0.08 | 1.04 ± 0.24 | 4.7 |
| 1e | 2.28 ± 0.36 | 1.03 ± 0.11 | 0.99 ± 0.53 | 2.2 |
| 1f | 4.56 ± 1.12 | 0.12 ± 0.02 | 0.54 ± 0.11 | 38 |
| 1g | 4.02 ± 1.04 | 0.41 ± 0.15 | 0.91 ± 0.03 | 9.8 |
| 1h | 8.11 ± 1.67 | 0.74 ± 0.06 | 0.70 ± 0.03 | 10.9 |
| 1i | 1.13 ± 0.15 | 0.79 ± 0.18 | 0.32 ± 0.13 | 1.4 |
| 1j | 3.29 ± 0.45 | 1.06 ± 0.08 | 1.55 ± 0.14 | 3.1 |
| 1k | 4.78 ± 0.18 | 1.30 ± 0.09 | 6.36 ± 1.04 | 3.7 |
| 1l | 53.79 ± 7.28 | 1.30 ± 0.28 | 53.79 ± 7.28 | 41.3 |
| 1m | 2.79 ± 0.85 | 1.56 ± 0.19 | 2.25 ± 0.13 | 1.8 |
| 1n | 1.28 ± 0.28 | 1.59 ± 0.11 | 0.26 ± 0.09 | 0.8 |
| 1o | 0.21 ± 0.09 | 1.59 ± 0.24 | 1.36 ± 0.23 | 0.13 |
| 1p | 4.64 ± 0.04 | 1.74 ± 0.08 | 3.16 ± 0.43 | 2.7 |
| 1q | 52.58 ± 8.62 | 2.15 ± 0.17 | 8.04 ± 0.71 | 24.5 |
| 1r | 51.23 ± 5.71 | 2.64 ± 0.01 | 10.20 ± 1.42 | 9.0 |
| 1s | 2.29 ± 0.56 | 2.81 ± 0.42 | 0.67 ± 0.15 | 0.8 |
| 1t | 25.95 ± 2.40 | 1.83 ± 0.27 | 7.72 ± 1.20 | 14.2 |
| 1u | 7.67 ± 0.71 | 0.59 ± 0.08 | 0.33 ± 0.01 | 13.0 |
| 1v | >10 | 1.57 ± 0.15 | 2.41 ± 0.09 | >6.4 |
| 1w | 25.3 ± 4.57 | 1.72 ± 0.12 | 4.26 ± 0.41 | 14.7 |
| 1x | 2.70 ± 0.42 | 0.81 ± 0.09 | 0.45 ± 0.07 | 3.33 |
| 1y | 0.69 ± 0.17 | 0.55 ± 0.05 | 0.67 ± 0.04 | 1.25 |
| 1z | 0.70 ± 0.42 | 0.77 ± 0.08 | 0.35 ± 0.12 | 0.91 |
| 2a | 8.00 ± 0.58 | 0.89 ± 0.25 | 1.86 ± 0.39 | 9 |
| 2b | 0.72 ± 0.03 | 0.33 ± 0.02 | 0.46 ± 0.09 | 2.2 |
| 2c | 6.86 ± 0.07 | 1.26 ± 0.12 | 1.78 ± 0.45 | 5.4 |
| 2d | 5.78 ± 1.30 | 1.39 ± 0.07 | 0.95 ± 0.05 | 4.1 |
| 2e | 6.10 ± 1.34 | 1.77 ± 0.02 | 3.59 ± 0.24 | 3.4 |
| 2f | 1.66 ± 0.45 | 1.82 ± 0.66 | 1.45 ± 0.52 | 0.91 |
| 2g | 27.85 ± 6.37 | 2.46 ± 0.19 | 9.11 ± 1.47 | 11.3 |
| 2h | 5.15 ± 0.65 | 3.94 ± 0.43 | 6.73 ± 0.88 | 1.3 |
| 2i | 2.63 ± 0.50 | 3.11 ± 0.62 | 3.81 ± 0.06 | 0.84 |
| 2j | 8.67 ± 1.31 | 3.39 ± 0.34 | 7.99 ± 0.83 | 2.6 |
| 2k | 8.18 ± 2.67 | 3.69 ± 0.65 | 5.12 ± 0.32 | 2.2 |
| 2l | 135.55 ± 16.45 | 6.63 ± 0.83 | 5.14 ± 0.27 | 20.4 |
| 2m | 1.30 ± 0.18 | 2.56 ± 0.17 | 4.19 ± 0.71 | 0.51 |
| 2n | 4.07 ± 0.24 | 7.59 ± 1.11 | 3.49 ± 0.49 | 0.54 |
| 3a | 4.75 ± 0.57 | 8.38 ± 0.72 | 2.07 ± 0.09 | 0.57 |
| 3b | 18.60 ± 0.73 | 5.11 ± 0.84 | 2.15 ± 0.09 | 3.6 |
| 3c | 3.40 ± 0.54 | 1.65 ± 0.22 | 0.68 ± 0.16 | 2.1 |
| 3d | 7.84 ± 0.70 | 3.15 ± 0.28 | 1.49 ± 0.15 | 2.5 |
| 3e | 15.53 ± 1.91 | 3.40 ± 0.52 | 2.19 ± 0.04 | 4.7 |
| 3f | 7.42 ± 1.38 | 5.08 ± 0.62 | 0.86 ± 0.04 | 1.5 |
| 3g | 0.32 ± 0.03 | 3.03 ± 0.18 | 0.38 ± 0.08 | 0.11 |

*Ki values represent an average ± S.E. of at least three independent experiments.

Some compounds of Table 2 display unexpected selectivity toward FABP5 compared to FABP3.

### Example 5. Anticancer Activity of TANE-Based FABP5 Inhibitors

Cell Lines

PC-3 (human metastatic prostate cancer), HepG2 (human liver cancer) and WI-38 (human normal lung fibroblast) cells were obtained from American Type Culture Collection (ATCC). These cells were grown in Roswell Park Memorial Institute (RPMI) 1640 Medium (Gibco-Thermo Fisher Scientific) supplemented with 10% FBS (Corning-Thermo Fisher Scientific) and 100 units/mL of penicillin/streptomycin (Gibco-Thermo Fisher Scientific) in a humidified incubator containing 95% air and 5% CO2.

Cytotoxicity (MTT) Assay

Cytotoxicity of TAME-based FABP5 inhibitors was determined using the MTT colorimetric assay (Sigma-Aldrich). PC-3 (2500 cells/well) cells in 100 μl/well with RPMI 1640 supplemented with 1% or 10% FBS were seeded into 96-well plates (Corning, Inc., Corning, NY, USA) and incubated for 24 hours at 37° C. In the cases of HepG2 and WI38, 10,000 cells/well and 7,500 cells/well were seeded, respectively. After removal of the previous medium, the cells were treated with RPMI 1640 supplemented with 1% FBS containing designed concentrations of TAMEs. All compounds for in vitro experimentation were dissolved in a vehicle of DMSO at a final concentration of 0.1% (a few compounds were conducted at up to 1.0% due to insufficient water solubility). After a 72-hour incubation period, the previous medium was removed. Then the cells were treated with 100 μl/well MTT (0.5 mg/mL in serum-free RPMI 1640) and incubated for 4 hours at 37° C. After the incubation period, the supernatant was carefully removed. The remaining formazan was solubilized using 100 μl/well DMSO, and the absorbance was read at 570 nm in a VersaMax Microplate Reader (Molecular Devices, Sunnyvale, CA). Each experiment was repeated three times. The 50% growth inhibitory concentration (IC50) of compounds was calculated by GraphPad Prism (version 8.0.2) to evaluate the drug sensitivity. Data are represented as means±SEM.

pkCSM ADMET Property Prediction

pkCSM uses graph-based signatures to develop predictive models of central ADMET properties for drug development and has been extensively used by medicinal chemists (Pires, D. E. et al. 2015). pkCSM is accessible at the web server http://structure.bioc.cam.ac.uk/pkcsm.

TABLE 3
Anticancer activity of novel TAME-based FABP5 inhibitors and
pkCSM predictions on hERG toxicity and mutagenicity (AMES)

| | PC3 | HepG2 | WI-38 | | | |
|---|---|---|---|---|---|---|
| Cmpd | (IC₅₀ μM) | (IC₅₀ μM) | (IC₅₀ μM) | hERG I | hERG II | AMES |
| 1f | 11.8 | 66.3 | >100 | NO | NO | NO |
| 1o | 8.69 | 9.43 | >100 | NO | NO | NO |
| 1e | 6.79 | 9.41 | 27.3 | NO | NO | YES |
| 1c | 6.26 | 7.99 | 88.3 | NO | NO | NO |
| 1g | 4.53 | 8.87 | 49.8 | NO | NO | NO |
| 2c | 10.1 | 16.4 | 52.1 | NO | NO | NO |
| 1m | 7.25 | 11.2 | 49.5 | NO | YES | NO |
| 1h | 4.93 | 7.89 | 24.0 | NO | NO | NO |
| 1n | 20.7 | 23.5 | 59.4 | NO | NO | NO |
| 1q | 11.3 | 12.4 | 20.3 | NO | NO | NO |
| 2a | 15.2 | 7.84 | 24.1 | NO | NO | NO |
| 1l | 48.9 | 45.5 | 63.7 | NO | NO | NO |
| 3g | 10.6 | 36.9 | 54.2 | NO | NO | NO |
| 3f | 0.95 | 1.26 | 1.60 | NO | NO | NO |
| 3c | 15.0 | 13.8 | 22.2 | NO | YES | YES |

### Example 6. Synergistic Anticancer Activity Through Combination of a Taxane and FABP5 Inhibitors

Cell-Lines

PC3 cells were obtained from American Type Culture Collection (ATCC; CRL-1435; Manassas, VA) and were authenticated by the ATCC human short-tandem repeat profiling cell authentication service. DU-145 and 22Rv1 cells were also obtained from ATCC (HTB-81 and CRL-2505, respectively; ATCC). PC3, DU-145, and 22Rv1 cell-lines were each grown in Roswell Park Memorial Institute 1640 (RPMI 1640) (Gibco-Thermo Fisher Scientific, Gaithersburg MD) supplemented with 10% fetal bovine serum (FBS) (Gemini Bio-Products, West Sacramento, CA) and 100 units/mL of penicillin/streptomycin (Gibco-Thermo Fisher Scientific) in a humidified incubator containing 95% air and 5% C02. WI-38 cells were obtained from ATCC (CCL-75). WI-38 cells were grown in Dulbecco's modified Eagle's medium (DMEM) (Gibco-Thermo Fisher Scientific) supplemented with 10% FBS and 100 units/mL of penicillin/streptomycin in a humidified incubator containing 95% air and 5% C02. RWPE-1 cells were purchased from ATCC (CRL-11609). RWPE-1 cells were grown in keratinocyte serum-free media (K-SFM) (Gibco-Thermo Fisher Scientific) supplemented with 25 mg of bovine pituitary extract (BPE), 1 mg of recombinant human epidermal growth factor (EGF), and 100 units/mL of penicillin/streptomycin in a humidified incubator containing 95% air and 5% C02.

Cytotoxicity Assays

Cytotoxicity of 1y, 1w, docetaxel, and cabazitaxel (both individually, and in combination) were determined using the 3-(4,5-dimethylthiazol-2-yl)-2,5 diphenyl tetrazolium bromide (MTT) colorimetric assay (Sigma-Aldrich). PC3 (2500 cells/well), DU-145, 22Rv1, WI-38 (5000 cells/well), and RWPE-1 (10000 cells/well) cells were seeded into 96-well plates and incubated for 24 hours at 37° C. in their respective media (PC3/DU-145/22Rv1 cells utilized RPMI 1640; WI-38 cells utilized DMEM; RWPE-1 cells utilized K-SFM). PC3, DU-145, and 22Rv1 cells were treated with RPMI 1640 supplemented with 1% FBS containing 0.1 μM to 100 μM 1y or 1w, and/or 0.003 nM to 300 nM docetaxel or cabazitaxel (both individually, or in combination with 1y or 1w). WI-38 cells were treated with DMEM supplemented with 1% FBS containing 0.1 μM to 100 μM 1y or 1w. RWPE-1 cells were treated with K-SFM supplemented with 25 mg of BPE and 1 mg of recombinant human EGF containing 0.1 μM to 100 μM 1y or 1w. All drugs for in vitro experimentation were dissolved in a vehicle of DMSO at a final concentration of 0.1%. Additionally, the appropriate treatment media for each cell-line supplemented with 0.1% DMSO or 1% sodium dodecyl sulfate was used as either a positive or negative control, respectively. After a 72-hour incubation period, cells were washed with PBS and treated with MTT (0.5 mg/mL in serum-free RPMI 1640, serum-free DMEM, or K-SFM) for 4 hours. The cells were subsequently solubilized using DMSO and the absorbance was read at 562 nm in an F5 Filtermax Multi-Mode Microplate Reader (Molecular Devices, Sunnyvale, CA).

Analysis of Combined Drug Effects

Synergism between docetaxel/cabazitaxel and 1y or 1w was determined through the combination-index (CI) method using the median-effect principle of mass-action law, derived from Chou and Talalay (Chou, T. C. (2006) using ComboSyn software. Briefly, individual drug concentrations that result in the desired fraction of cells affected (Fa) were measured (ie, the concentration of 1y, 1w, docetaxel, or cabazitaxel, which result in the same fraction of cells killed). The concentration resulting in the desired Fa (eg, Fa=0.5 represents 50% of cells effected) for each drug was plotted on an XYaxis, and a straight line drawn to connect the data points. The coadministration of two drugs that achieves the same desired Fa was then plotted on the same axis. Data points that fall above the line (CI>1) represent antagonism, data points that fall on the line (CI=1) represent an additive interaction, and data points that fall below the line (CI<1) represent synergism.

TABLE 4
Synergy analysis of ly or 1w and docetaxel combinations in
PC3, DU-145, and 22Rv1 cell lines

| Cell | 1y | 1w | Docetaxel | Fa | CI | |
|---|---|---|---|---|---|---|
| line | (μM) | (μM) | (nM) | value | value | Relationship |
| PC3 | 7.5 | — | 0.03 | 0.786 | 0.897 | synergistic |
| PC3 | 7.5 | — | 0.3 | 0.950 | 0.009 | synergistic |
| PC3 | 7.5 | — | 3.0 | 0.992 | 0.160 | synergistic |
| PC3 | — | 1.0 | 0.03 | 0.402 | 0.710 | synergistic |
| PC3 | — | 1.0 | 0.3 | 0.432 | 0.889 | synergistic |
| PC3 | — | 1.0 | 3.0 | 0.700 | 0.394 | synergistic |
| DU-145 | 7.5 | — | 0.03 | 0.934 | 1.302 | — |
| DU-145 | 7.5 | — | 0.3 | 0.963 | 0.968 | synergistic |
| DU-145 | 7.5 | — | 3.0 | 0.998 | 0.123 | synergistic |
| DU-145 | — | 1.0 | 0.03 | 0.516 | 4.473 | — |
| DU-145 | — | 1.0 | 0.3 | 0.676 | 2.697 | — |
| DU-145 | — | 1.0 | 3.0 | 0.935 | 0.445 | synergistic |
| 22Rv1 | 7.5 | — | 0.03 | 0.795 | 3.846 | — |
| 22Rv1 | 7.5 | — | 0.3 | 0.904 | 0.003 | synergistic |
| 22Rv1 | 7.5 | — | 3.0 | 0.999 | 0.005 | synergistic |
| 22Rv1 | — | 1.0 | 0.03 | 0.457 | 1.673 | — |
| 22Rv1 | — | 1.0 | 0.3 | 0.744 | 0.373 | synergistic |
| 22Rv1 | — | 1.0 | 3.0 | 0.905 | 0.267 | synergistic |

Abbreviations: Fa, fraction of cells affected; CI, combination-index.

TABLE 5
Synergy analysis of ly or 1w and cabazitaxel combinations in
PC3, DU-145, and 22Rv1 cell lines

| Cell- | 1y | 1w | cabazitaxel | Fa | CI | |
|---|---|---|---|---|---|---|
| line | (μM) | (μM) | (nM) | value | value | Relationship |
| PC3 | 7.5 | — | 0.03 | 0.998 | 0.0001 | synergistic |
| PC3 | 7.5 | — | 0.3 | 0.999 | 0.0001 | synergistic |
| PC3 | 7.5 | — | 3.0 | 0.999 | 0.0001 | synergistic |
| PC3 | — | 1.0 | 0.03 | 0.379 | 0.899 | synergistic |
| PC3 | — | 1.0 | 0.3 | 0.420 | 0.942 | synergistic |
| PC3 | — | 1.0 | 3.0 | 0.853 | 0.373 | synergistic |
| DU-145 | 7.5 | — | 0.03 | 0.829 | 0.709 | synergistic |
| DU-145 | 7.5 | — | 0.3 | 0.885 | 0.322 | synergistic |
| DU-145 | 7.5 | — | 3.0 | 0.939 | 0.136 | synergistic |
| DU-145 | — | 1.0 | 0.03 | 0.464 | 0.449 | synergistic |
| DU-145 | — | 1.0 | 0.3 | 0.716 | 0.300 | synergistic |
| DU-145 | — | 1.0 | 3.0 | 0.882 | 0.194 | synergistic |
| 22Rv1 | 7.5 | — | 0.03 | 0.777 | 0.707 | synergistic |
| 22Rv1 | 7.5 | — | 0.3 | 0.899 | 0.396 | synergistic |
| 22Rv1 | 7.5 | — | 3.0 | 0.998 | 0.003 | synergistic |
| 22Rv1 | — | 1.0 | 0.03 | 0.577 | 0.731 | synergistic |
| 22Rv1 | — | 1.0 | 0.3 | 0.728 | 0.549 | synergistic |
| 22Rv1 | — | 1.0 | 3.0 | 0.823 | 0.659 | synergistic |

Abbreviations: Fa, fraction of cells affected; CI, combination-index.

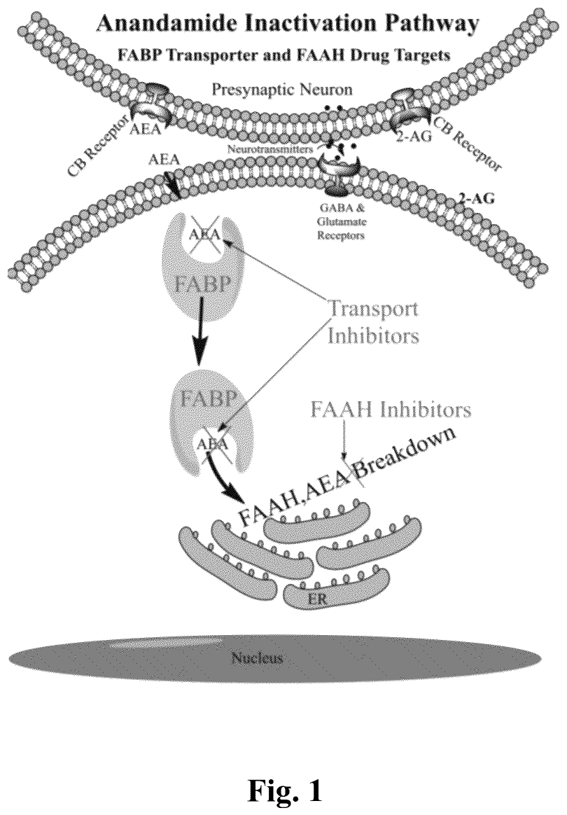

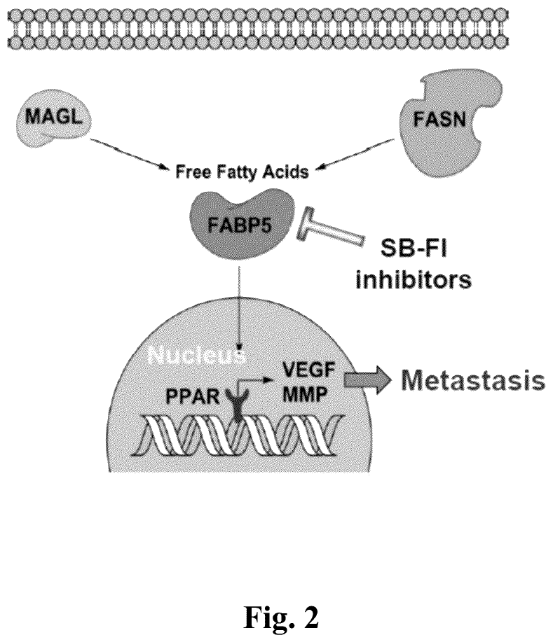

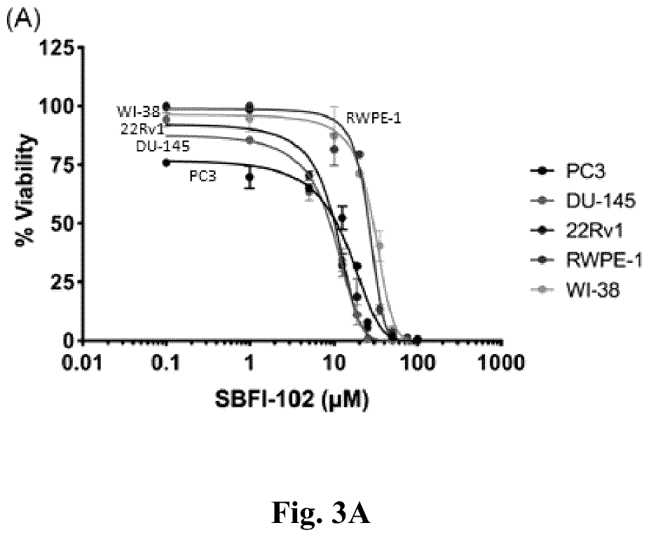

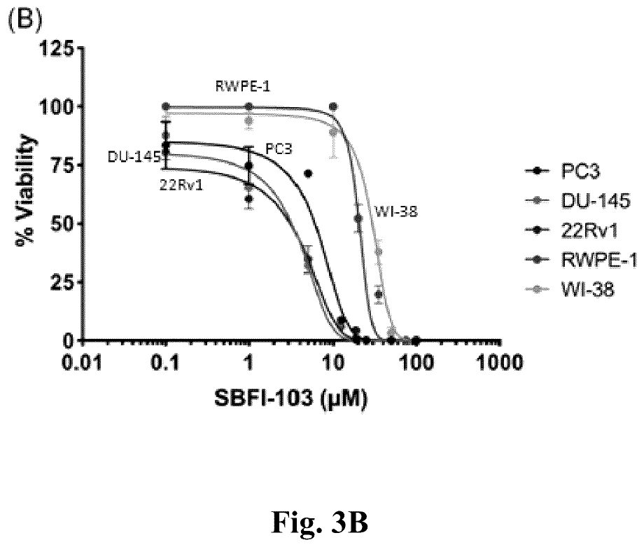

The cytotoxic effects of 1y (SBFI-102) (FIG. 3A) and 1w (SBFI-103) (FIG. 3B) were assessed in human-derived PC3, DU-145, and 22Rv1 cells that express FABP5 (Kawaguchi, K. et al. 2016). 1y (SBFI-102) and 1w (SBFI-103) produced dose-dependent cytotoxicity in each cell-line tested: PC3 cells with IC50 values of 11.4 and 6.3 μM, respectively; DU-145 cells with IC50 values of 8.9 and 3.3 μM, respectively; and 22Rv1 cells with IC50 values of 10.1 and 3.1 μM, respectively. Both 1y (SBFI-102) and 1w (SBFI-103) showed less cytotoxicity in RWPE-1 cells (a normal prostate cell-line), producing IC50 values of 26.0 and 20.6 μM, respectively (FIG. 3A,B). Both 1y (SBFI-102) and 1w (SBFI-103) showed less cytotoxicity in WI-38 cells (a normal lung cell-line), producing IC50 values of 29.4 and 29.6 μM, respectively (FIG. 3A,B).

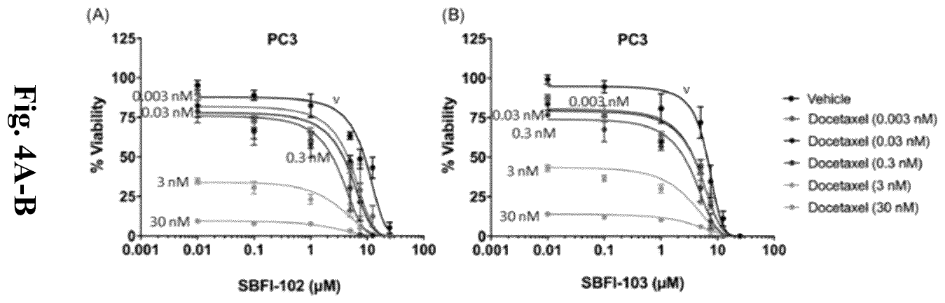

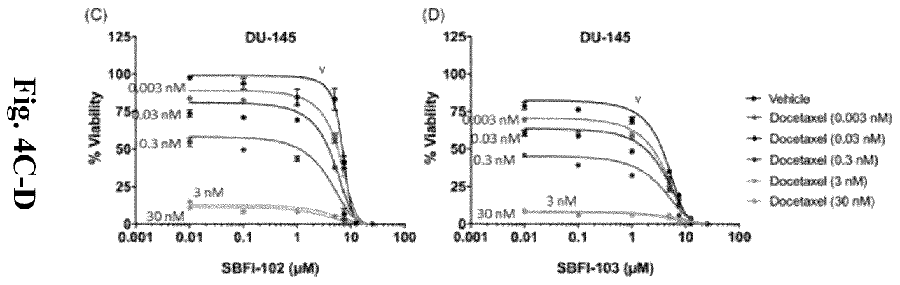

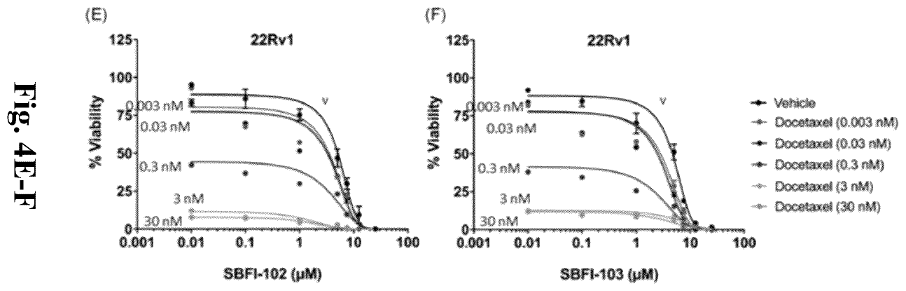

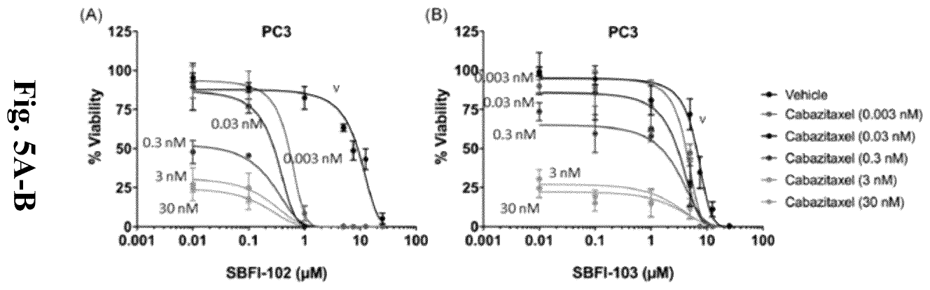

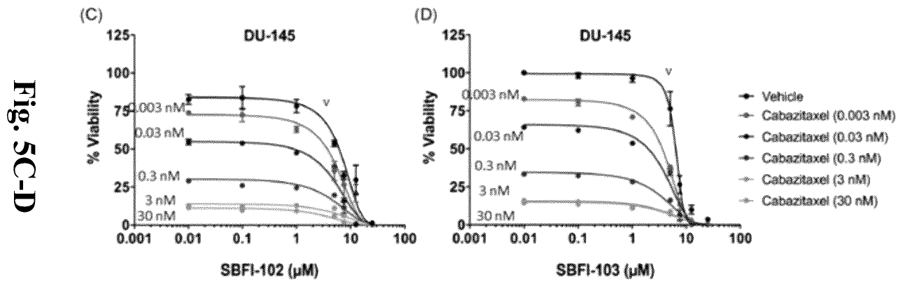

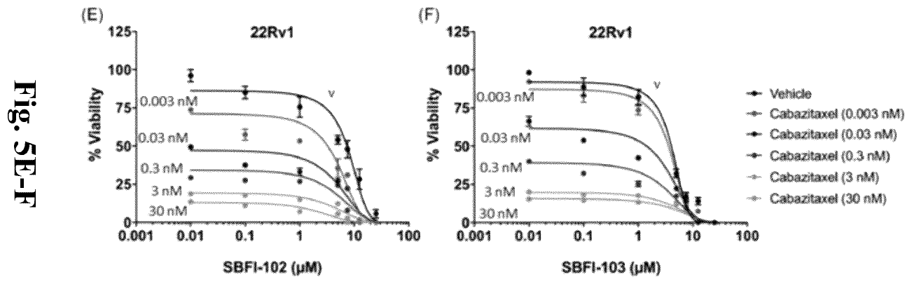

A combination of docetaxel or cabazitaxel with FABP5 inhibitors 1y (SBFI-102) or 1w (SBFI-103) resulted in greater cytotoxicity in PC3, DU-145, and 22Rv1 cells than each drug when administered independently (FIGS. 4 and 5). Synergistic relationships were observed between docetaxel and the FABP5 inhibitors in each cell-line (CI<1) (Table 4). Synergistic relationships between cabazitaxel and the FABP5 inhibitors were also observed (Table 5).

Animals

Male BALB/c nude mice (BALB/cOlaHsd-Foxn1nu, 20-30 g, 7-8 weeks old) (Envigo RMS Inc, Indianapolis, IN) were used for all experiments. Animals were housed individually at room temperature and were kept on a 12:12-hour light:dark cycle with access to food and water ad libitum. Euthanasia was carried out utilizing CO2 asphyxiation. All of the experiments were approved by the Stony Brook University Animal Care and Use Committee.

Subcutaneous Tumor Implantation

Male BALB/c nude mice were subcutaneously inoculated with PC3 cells. Briefly, cells (1×10⁶ per mouse) were resuspended in 100 μL of a 1:1 mixture of phosphate-buffered saline (PBS):Matrigel (Corning Inc, Corning, NY) and implanted into a single dorsal lateral flank using a 21G needle. Tumor length (L) and tumor width (W) were measured twice weekly using digital calipers, and tumor volume (V) was calculated as (V=[L×W2]/2). When tumor volume reached approximately 150 to 200 mm3, animals were grouped and drug administration commenced. Humane endpoints for all animals were as follows: animals carrying a tumor burden greater than 35 days, body weight (which was recorded twice weekly) decreasing by greater than 15%, tumor ulceration, paralysis, failure to groom, bleeding, respiratory distress, and/or tumor volume reaching 1500 mm3.

Drug Administration

1y (SBFI-102), 1w (SBFI-103), and docetaxel were each reconstituted in a 1:1:8 vehicle consisting of dimethyl sulfoxide (DMSO) (Thermo Fisher Scientific, Hampton, NH):Cremaphor-EL (Sigma-Aldrich):saline. 1y and 1w were administered via intraperitoneal injection (ip) using a 27G needle at 20 mg/kg daily. Docetaxel was administered i.p. at 5 or 10 mg/kg weekly. All drugs were administered in a volume of 10 μL/g body weight.

Quantification and Statistical Analysis

All data were obtained from at least three independent experiments and then values described in each figure legend depict each independent trial or animal. Data for all in vivo experiments were analyzed using a one-way analysis of variance with the Tukey post hoc test (GraphPad Prism, version 8.0.2). Data are represented as means±SEM and P<0.05 was considered statistically significant. The degree of significance is indicated in each figure legend.

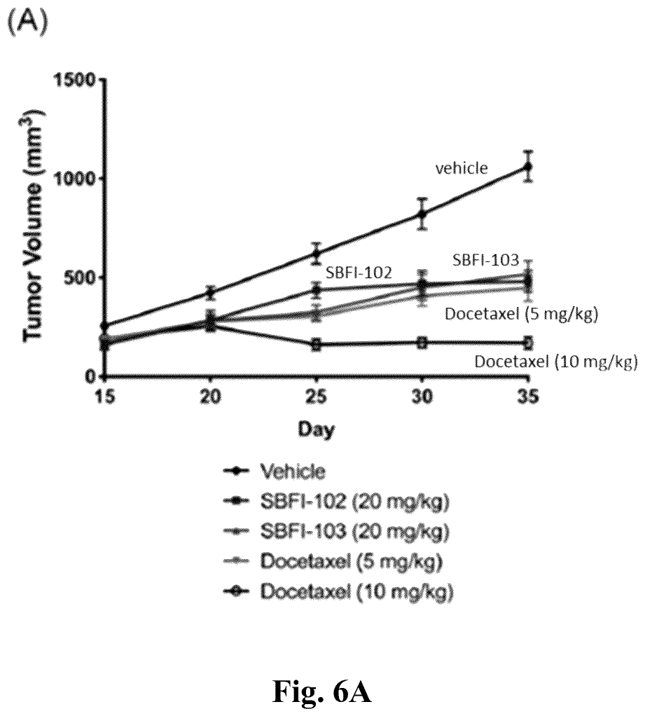

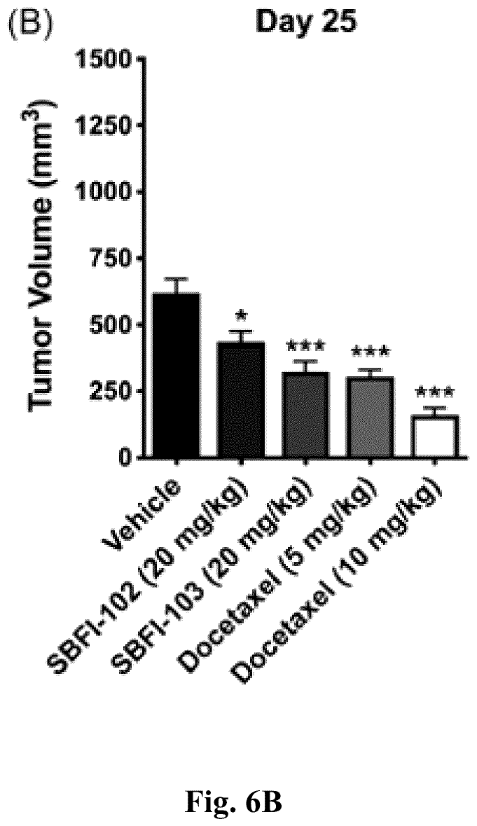

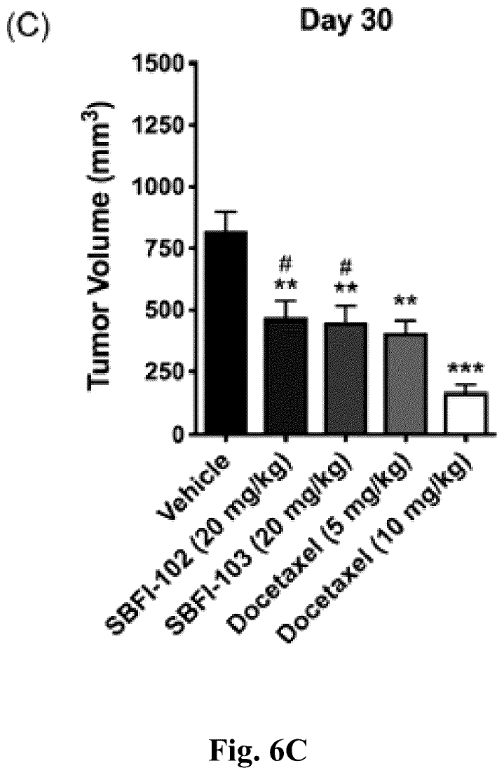

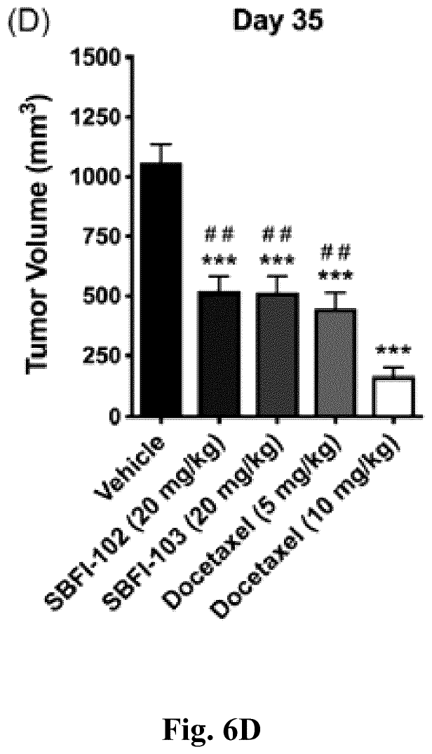

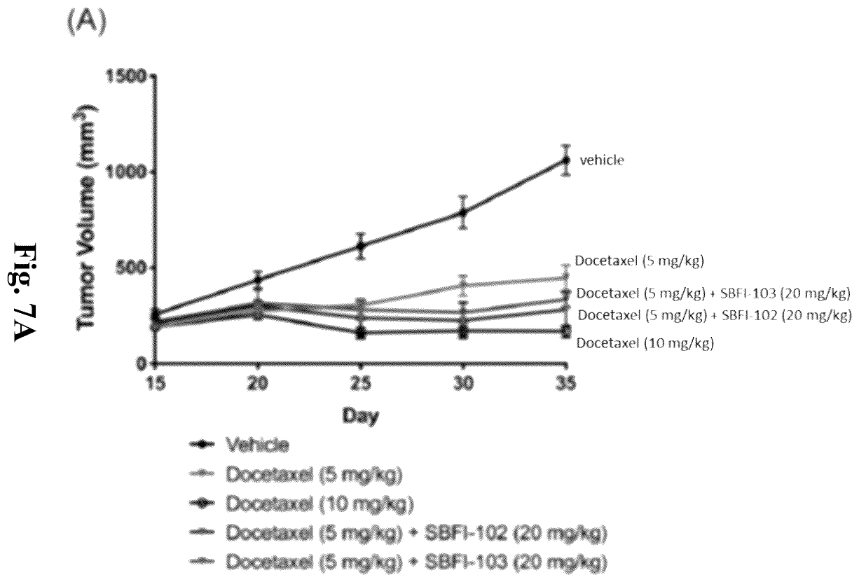

Administration of 1y (SBFI-102) or 1w (SBFI-103) (20 mg/kg, ip, once daily) significantly reduced tumor growth (FIG. 6A). Similarly, administration of docetaxel (5 or 10 mg/kg, ip, once weekly) reduced tumor growth, with the 5 mg/kg dose producing similar inhibition of tumor growth as observed with the FABP5 inhibitors, while the 10 mg/kg dose produced near complete inhibition of growth (FIG. 6A-D).

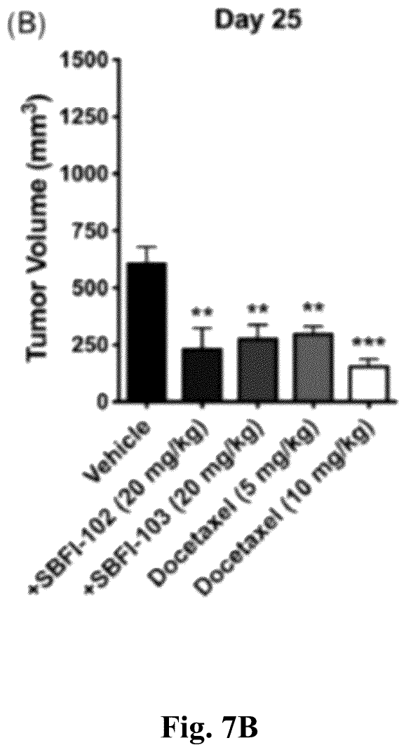

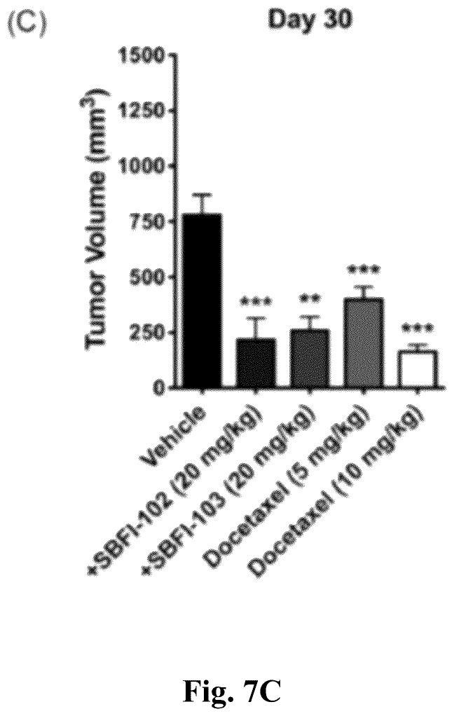

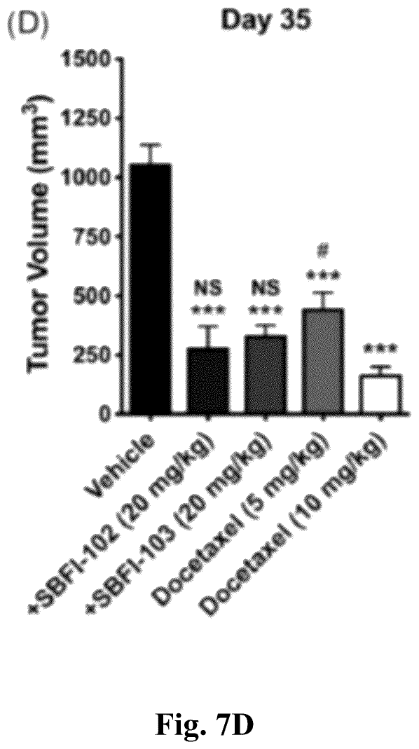

To determine whether 1y (SBFI-102) and 1w (SBFI-103) enhance the tumor suppressive effects of docetaxel, we administered the FABP5 inhibitors in combination with the submaximal dose of docetaxel (5 mg/kg). Consistent with the in vitro efficacy data, coadministration of docetaxel with 1y (SBFI-102) or 1w (SBFI-103) produced greater inhibition of tumor growth than treatment with each compound alone, with effects that were comparable in magnitude to the 10 mg/kg docetaxel dose (FIG. 7A-D).

Discussion

Described herein are novel α-, γ- and ε-truxillic acid monoesters (TAMEs) that are highly promising inhibitors of fatty acid binding protein 5 (FABP5). These compounds effectively bind to FABP5, blocking the intercellular shuttling of endocannabionds, thereby increasing the endogenous levels of anandamide by circumventing degradation by the fatty acid amide hydrolase (FAAH) enzyme. The resulting increase in extracellular anandamide which triggers activation of the cannabinoid receptor type 1 (CB-1) pathway, leading to the relief of nociceptive, neurogenic and inflammatory pain. Thus, those novel TAMEs are anticipated to serve a next-generation agents for chronic pains.

In addition to the pain management, those selective FABP5 inhibitors act as anticancer agents. FABP5 is an intracellular lipid carrier whose expression is upregulated in metastatic pancreatic cancer (PCa) and increases cell growth, invasion, and tumor formation. Thus, we assessed whether FABP5 inhibitors synergize with clinically used taxanes to induce cytotoxicity in vitro and attenuate tumor growth in vivo. Herein, we show that some TAMEs produced cytotoxicity in the PCa cells. Coincubation of the PCa cells with FABP5 inhibitors and docetaxel or cabazitaxel produced synergistic cytotoxic effects in vitro. Treatment of mice with FABP5 inhibitors reduced tumor growth and a combination of FABP5 inhibitors with a submaximal dose of docetaxel reduced tumor growth to a larger extent than treatment with each drug alone. Thus, FABP5 inhibitors increase the cytotoxic and tumor-suppressive effects of taxanes in PCa cells. The ability of these drugs to synergize could permit more efficacious antitumor activity while allowing for taxane anticancer drugs to be lowered, potentially mitigating taxane-resistance.

Additionally, the anticipated “side effect” of FABP5 inhibitors is its anti-inflammatory and antinociceptive effects, which would be a welcome “side effect” for cancer patients. The novel TAMEs of this invention are selective to FABP5 and against FABP3 which is expressed in the heart muscle tissues and its inhibition could cause arrhythmia.

### REFERENCES

Ahmad, I., Mui, E., Galbraith, L., Patel, R., Tan, E. H., Salji, M., Rust, A. G., Repiscak, P., Hedley, A., Markert, E., Loveridge, C., van der Weyden, L., Edwards, J., Sansom, O. J., Adams, D. J., Leung, H. Y. (2016) Sleeping Beauty screen reveals Pparg activation in metastatic prostate cancer. Proc Natl Acad Sci USA, 113, 8290-8295.
Ahn, K., Johnson, D. S., and Cravatt, B. F. (2009) Fatty acid amide hydrolase as a potential therapeutic target for the treatment of pain and CNS disorders. Expert opinion on drug discovery 4, 763-784.
Alwarawrah, Y., Hughes, P., Loiselle, D., Carlson, D. A., Darr, D. B., Jordan, J. L., Xiong, J., Hunter, L. M., Dubois, L. G., Thompson, J. W., Kulkarni, M. M., Ratcliff, A. N., Kwiek, J. J., Haystead, T. A. (2016) Fasnall, a Selective FASN Inhibitor, Shows Potent Anti-tumor Activity in the MMTV-Neu Model of HER2(+) Breast Cancer, Cell Chem Biol, 23, 678-688.
Baba, Y., Funakoshi, T., Mori, M., Emoto, K., Masugi, Y., Ekmekcioglu, S., Amagai, M., Tanese, K. (2017) Expression of monoacylglycerol lipase as a marker of tumour invasion and progression in malignant melanoma. J Eur Acad Dermatol Venereol, 31, 2038-2045.
Bao, Z., Malki, M. I., Forootan, S. S., Adamson, J., Forootan, F. S., Chen, D., Foster, C. S., Rudland, P. S., Ke, Y. (2013) A novel cutaneous Fatty Acid-binding protein-related signaling pathway leading to malignant progression in prostate cancer cells. Genes Cancer 4, 297-314.
Berger, W. T., Ralph, B. P., Kaczocha, M., Sun, J., Balius, T. E., Rizzo,
R. C., Haj-Dahmane, S., Ojima, I., and Deutsch, D. G. (2012) Targeting fatty acid binding protein (FABP) anandamide transporters—a novel strategy for development of anti-inflammatory and anti-nociceptive drugs. PLoS One 7, e50968.
Chmurzynska, A. et al. (2006) Chmurzynska A (2006) The multigene family of fatty acid-binding proteins (FABPs): function, structure and polymorphism. J Appl Genet 47, 39-48.
Chou, T. C. (2006) Theoretical basis, experimental design, and computerized simulation of synergism and antagonism in drug combination studies. Pharmacol Rev. 58, 621-681.
Deutsch, D. G. (2016) A personal retrospective: elevating anandamide (AEA) by targeting fatty acid amide hydrolase (FAAH) and the fatty acid binding proteins (FABPs). Front. Pharmacol. 7, 370.
Forootan, F. S., Forootan, S. S., Malki, M. I., Chen, D., Li, G., Lin, K., Rudland, P. S., Foster, C. S., Ke, Y. (2014) The expression of C-FABP and PPARgamma and their prognostic significance in prostate cancer. Int J Oncol 44, 265-275.
Fujita, K., Kume, H., Matsuzaki, K., Kawashima, A., Ujike, T., Nagahara, A., Uemura, M., Miyagawa, Y., Tomonaga, T., Nonomura, N. (2017) Proteomic analysis of urinary extracellular vesicles from high Gleason score prostate cancer. Scientific reports 7, 42961.
Furuhashi, M. and Hotamisligil, G. S. (2008) Fatty acid-binding proteins: role in metabolic diseases and potential as drug targets. Nat Rev Drug Discov 7, 489-503.
Her, N. G., Jeong, S. I., Cho, K., Ha, T. K., Han, J., Ko, K. P., Park, S. K., Lee, J. H., Lee, M. G., Ryu, B. K., Chi, S. G. (2013) PPARdelta promotes oncogenic redirection of TGF-beta1 signaling through the activation of the ABCA1-Cavl pathway, Cell Cycle. 12, 1521-1535.
Howlett, A. C., Reggio, P. H., Childers, S. R., Hampson, R. E., Ulloa, N. M., and Deutsch, D. G. (2011) Endocannabinoid tone versus constitutive activity of cannabinoid receptors. British Journal of Pharmacology 163, 1329-1343.
Kaczocha, M., Glaser, S. T., and Deutsch, D. G. (2009) Identification of intracellular carriers for the endocannabinoid anandamide. Proc Natl Acad Sci USA 106, 6375-6380.
Kaczocha, M., Vivieca, S., Sun, J., Glaser, S. T., and Deutsch, D. G. (2012) Fatty acid-binding proteins transport N-acylethanolamines to nuclear receptors and are targets of endocannabinoid transport inhibitors. J. Biol. Chem. 287, 3415-3424.
Kawaguchi, K., Kinameri, A., Suzuki, S., Senga, S., Ke, Y., Fujii, H. (2016) The cancer-promoting gene fatty acid-binding protein 5 (FABP5) is epigenetically regulated during human prostate carcinogenesis. Biochem J. 473, 449-461.
Levi, L., Wang, Z., Doud, M. K., Hazen, S. L., Noy, N. (2015) Saturated fatty acids regulate retinoic acid signalling and suppress tumorigenesis by targeting fatty acid-binding protein 5, Nat Commun. 6, 8794.
Mallet, C., Dubray, C., and Dualé, C. (2016) FAAH inhibitors in the limelight, but regrettably. Int. J. Clin. Pharmacol. Ther. 54, 498-501.
Nomura, D. K., Lombardi, D. P., Chang, J. W., Niessen, S., Ward, A. M.,
Long, J. Z., Hoover, H. H., Cravatt, B. F. (2011) Monoacylglycerol lipase exerts dual control over endocannabinoid and fatty acid pathways to support prostate cancer. Chem Biol 18, 846-856.
Nomura, D. K., Long, J. Z., Niessen, S., Hoover, H. S., Ng, S. W., Cravatt, B. F. (2010) Monoacylglycerol lipase regulates a fatty acid network that promotes cancer pathogenesis. Cell 140, 49-61.
Pires, D. E., Blundell, T. L., Ascher, D. B. (2015) pkCSM: Predicting Small-Molecule Pharmacokinetic and Toxicity Properties Using Graph-Based Signatures, J Med Chem 58, 4066-4072.
Regula, N., Haggman, M., Johansson, S., Sorensen, J. (2016) Malignant lipogenesis defined by 11C-acetate PET/CT predicts prostate cancer-specific survival in patients with biochemical relapse after prostatectomy. Eur J Nucl Med Mol Imaging 43, 2131-2138.
Rossi, S., Graner, E., Febbo, P., Weinstein, L., Bhattacharya, N., Onody, T., Bubley, G., Balk, S., Loda, M. (2003) Fatty acid synthase expression defines distinct molecular signatures in prostate cancer. Mol Cancer Res 1, 707-715.
Schug, T. T., Berry, D. C., Shaw, N. S., Travis, S. N., Noy, N. (2007) Opposing effects of retinoic acid on cell growth result from alternate activation of two different nuclear receptors. Cell 129, 723-733.
Smathers, R. L. and Petersen, D. R. (2011) The human fatty acid-binding protein family: Evolutionary divergences and functions. Hum Genomics 5, 170-191.
Veerkamp, J. H. and Zimmerman, A. W. (2001) Fatty acid-binding proteins of nervous tissue. J Mol Neurosci 16, 133-142.

1. A compound having the structure: 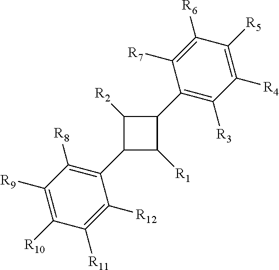
wherein
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I 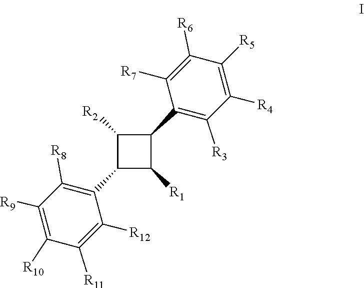
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O) OH and R₃, R₄, R₅, R₆, R₇, R₈, R₉, R₁₀, R₁₁ and R₁₂ are each H, then the other of R₁ or R₂ is other than —C(═O) OR₁₃ where R₁₃ is methyl, 2-propyl, pentyl, octyl, —CH₂C(O)CH₃, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl, or —C(═O)O-alkyl-R₁₄ where the alkyl is a branched C₂ alkyl and the R₁₄ is phenyl or the alkyl is a C₁ alkyl and the R₁₄ is phenyl, 4-methoxyphenyl, 4-fluorophenyl, 4-bromophenyl, or 9-fluorene,
wherein when one of R₁ or R₂ is —C(═O)OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene, 2-naphthalene, 2-phenylcyclohexyl, or —C(═O)O-alkyl-R₁₄ where the alkyl is a C₁ alkyl and the R₁₄ is 9-fluorene,
wherein when one of R₁ or R₂ is —C(═O)OH and R₄, R₅, R₆, R₇, R₉, R₁₀, R₁₁ and R₁₂ are each H and R₃ and R₈ are each —Cl or —Br, then the other of R₁ or R₂ is other than —C(═O)OR₁₃ where R₁₃ is 2-phenylcyclohexyl,
wherein when one of R₁ or R₂ is —C(═O)OH and R₄, R₅, R₆, R₉, R₁₀, and R₁₁ are each H and R₃, R₇, R₈ and R₁₂ are each —Cl, then the other of R₁ or R₂ is other than —C(═O)OR₁₃ where R₁₃ is 2-phenylcyclohexyl,
wherein when one of R₁ or R₂ is —C(═O)OH and R₃, R₄, R₆, R₇, R₈, R₉, R₁₁, and R₁₂ are each H and R₅ and R₁₀ are each —OH, then the other of R₁ or R₂ is other than —C(═O)OR₁₃ where R₁₃ is 1-naphthalene,
wherein when one of R₁ or R₂ is —C(═O)OH and R₃, R₆, R₇, R₈, R₁₁, and R₁₂ are each H, R₄ and R₉ are each OCH₃, and R₅ and R₁₀ are each —OH, then the other of R₁ or R₂ is other than —C(═O)OR₁₃ where R₁₃ is 1-naphthalene,
wherein when the compound has the stereochemistry of structure II 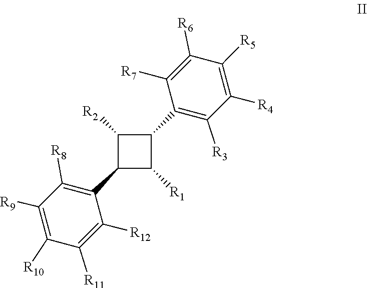
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O) OH and R₃, R₄, R₅, R₆, R₇, R₈, R₉, R₁₀, R₁₁ and R₁₂ are each H, then the other of R₁ or R₂ is other than —C(═O) OR₁₃ where R₁₃ is methyl, 2-propyl, pentyl, octyl, —CH₂C(O)CH₃, 1-naphthalene, 2-naphthalene or 2-methylphenyl, or —C(═O)O-alkyl-R₁₄ where the alkyl is a branched C₂ alkyl and the R₁₄ is phenyl,
wherein when the compound has the stereochemistry of structure III 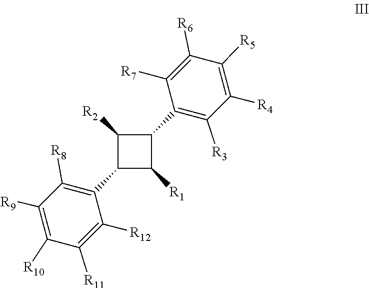
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

2. The compound of claim 1,
wherein
(a) one of R₁ or R₂ is —C(═O)OR₁₃,
wherein R₁₃ is cycloalkyl, aryl or heteroaryl; and
the other of R₁ or R₂ is —C(═O)OH;
(b) one of R₁ or R₂ is —C(═O)O-alkyl-R₁₄,
wherein R₁₄ is cycloalkyl, aryl or heteroaryl; and
the other of R₁ or R₂ is —C(═O)OH;
(c) one of R₁ or R₂ is —C(═O)O—(C₁₋₆ alkyl)-R₁₄,
wherein R₁₄ is cycloalkyl, aryl or heteroaryl; and
the other of R₁ or R₂ is —C(═O)OH; or
(d) one of R₁ or R₂ is —C(═O)O—CH₂—R₁₄,
wherein R₁₄ is cycloalkyl, aryl or heteroaryl; and
the other of R₁ or R₂ is —C(═O)OH.

3-5. (canceled)

6. The compound of claim 1, wherein
(a) R₁₃ or R₁₄ is a cycloalkyl that is substituted with a ring structure or fused to another ring structure; or
(b) R₁₃ or R₁₄ is an aryl or heteroaryl that is substituted with a ring structure or fused to another ring structure.

7. (canceled)

8. The compound of claim 1, wherein
(a) the aryl is substituted with a halogen, —OH, CN, —O(alkyl), amide, hydroxyaryl, aryl, a substituted aryl, heteroaryl or substituted heteroaryl;
(b) the heteroaryl is substituted with an aryl, amide, halogen, —OH, C₂-C₆ alkynyl, —O(alkyl), hydroxyaryl a substituted aryl, heteroaryl or substituted heteroaryl; and/or
(c) the cycloalkyl is i) substituted with a phenyl group, ii) fused with a phenyl group, or iii) fused with a benzo group.

9-10. (canceled)

11. The compound of claim 1, wherein the aryl is substituted with a F, Cl, Br, —OH, I, —NHC(O)CH₃, phenyl, o-hydroxyphenyl, triazolyl, C₂ alkynyl or —OCH₃, and/or wherein the heteroaryl is substituted with an F, Cl, Br, —OH, triazolyl, C₂ alkynyl, I, —NHC(O)CH₃ phenyl, o-hydroxyphenyl or —OCH₃.

12-19. (canceled)

20. The compound of claim 1, wherein the cycloalkyl is:

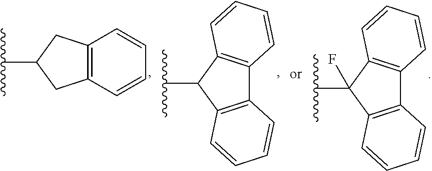

21. The compound of claim 1, wherein
(a) one of R₁ or R₂ is 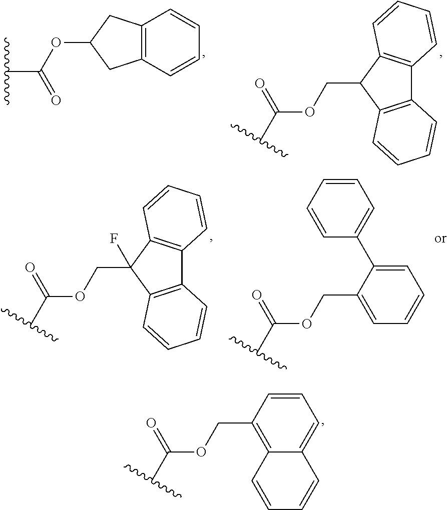
and
the other of R₁ or R₂ is —C(═O)OH;
(b) one of R₁ or R₂ is 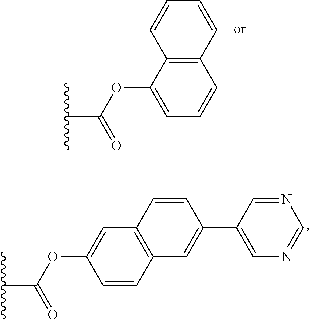 [SMILES: *C(=O)Oc1cccc2ccccc12 ; *C(=O)Oc1ccc2cc(-c3cncnc3)ccc2c1]
and
the other of R₁ or R₂ is —C(═O)OH;
(c) one of R₁ or R₂ is 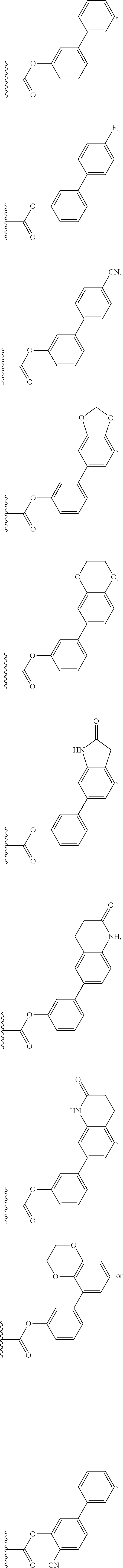
and
the other of R₁ or R₂ is —C(═O)OH; or
(d) one of R₁ or R₂ is 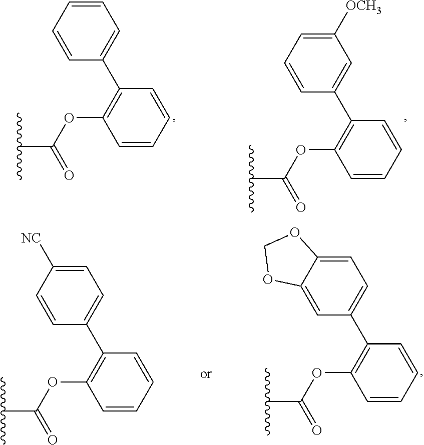
and
the other of R₁ or R₂ is —C(═O)OH.

22-24. (canceled)

25. The compound of claim 1, wherein
(a) R₃, R₄, R₅, R₆, R₇, R₈, R₉, R₁₀, R₁₁ and R₁₂ are each independently, —H, or —OR₁₅,
wherein R₁₅ is —H or C₁₋₁₀ alkyl;
(b) R₃, R₄, R₅, R₆, R₇, R₈, R₉, R₁₀, R₁₁ and R₁₂ are each independently, —H or —OCH₃,
(c) R₃, R₄, R₅, R₆, R₇, R₈, R₉, R₁₀, R₁₁ and R₁₂ are each —H;
(d) one of R₃, R₄, R₅, R₆, R₇, R₈, R₉, R₁₀, R₁₁ and R₁₂ is other than —H;
(e) two of R₃, R₄, R₅, R₆, R₇, R₈, R₉, R₁₀, R₁₁ and R₁₂ are other than —H;
(f) four of R₃, R₄, R₅, R₆, R₇, R₈, R₉, R₁₀, R₁₁ and R₁₂ are other than —H; or
(g) R₄, R₅, R₆, R₇, R₉, R₁₀, R₁₁ and R₁₂ are each —H and R₃ and R₈ are each —OCH₃.

26-31. (canceled)

32. The compound of claim 1,
wherein
(a) one of R₁ or R₂ is —C(═O)OH and the other of R₁ or R₂ is —C(═O)OR₁₃ or —C(═O)O-alkyl-R₁₄,
wherein
R₁₃ is cycloalkyl or aryl, and
R₁₄ is cycloalkyl or aryl; and
R₃, R₄, R₅, R₆, R₇, R₈, R₉, R₁₀, R₁₁ and R₁₂ are each independently, H or —OR₁₅,
wherein R₁₅ is H or C₁₋₁₀ alkyl;
(b) one of R₁ or R₂ is —C(═O)OH and the other of R₁ or R₂ is —C(═O)OR₁₃ or —C(═O)O-alkyl-R₁₄,
wherein
R₁₃ is cycloalkyl or aryl, and
R₁₄ is cycloalkyl or aryl; and
R₃, R₄, R₅, R₆, R₇, R₈, R₉, R₁₀, R₁₁ and R₁₂ are each H; or
(c) one of R₁ or R₂ is —C(═O)OH and the other of R₁ or R₂ is —C(═O)OR₁₃ or —C(═O)O-alkyl-R₁₄,
wherein
R₁₃ is cycloalkyl or aryl, and
R₁₄ is cycloalkyl or aryl; and
R₄, R₅, R₆, R₇, R₉, R₁₀, R₁₁ and R₁₂ are each —H and R₃ and R₈ are each —OCH₃.

33-34. (canceled)

35. The compound of claim 1 having the structure: 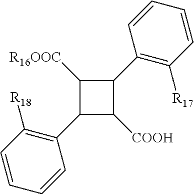
wherein
R₁₆ is cycloalkyl, alkylcycloalkyl, aryl or alkylaryl, and R₁₇ and R₁₈ are each independently, H or —OCH₃,
wherein when the compound has the stereochemistry of structure IV 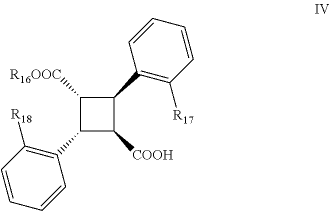
then
R₁₆ is cycloalkyl, alkylcycloalkyl, aryl or alkylaryl, and
R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, benzyl, methylbenzyl, 4-methoxybenzyl, 4-fluorobenzyl, 4-bromobenzyl, —CH₂-9-fluorene, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl,
wherein when R₁₇ and R₁₈ are each —OCH₃, then R₁₆ is other than 1-naphthalene, 2-naphthalene, 2-phenylcyclohexyl, or —CH₂-9-fluorene,
wherein when the compound has the stereochemistry of structure V 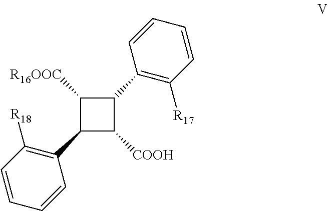
then
R₁₆ is cycloalkyl, alkylcycloalkyl, aryl or alkylaryl, and
R₁₇ and R₁₈ are each H or —OCH₃,
wherein when R₁₇ and R₁₈ are each H, then R₁₆ is other than methyl, 2-propyl, pentyl, octyl, —CH₂C(O)CH₃, methylbenzyl, 1-naphthalene, 2-naphthalene or 2-methylphenyl,
wherein when the compound has the stereochemistry of structure VI 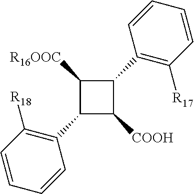
then
R₁₆ is cycloalkyl, alkylcycloalkyl, aryl or alkylaryl, and
R₁₇ and R₁₈ are each H or —OCH₃,
or an enantiomer or racemate thereof;
or a pharmaceutically acceptable salt thereof.

36. The compound of claim 35,
wherein
(a) R₁₆ is cycloalkyl, alkylcycloalkyl, aryl or alkylaryl;
(b) R₁₇ and R₁₈ are each H or —OCH₃;
(c) R₁₆ is 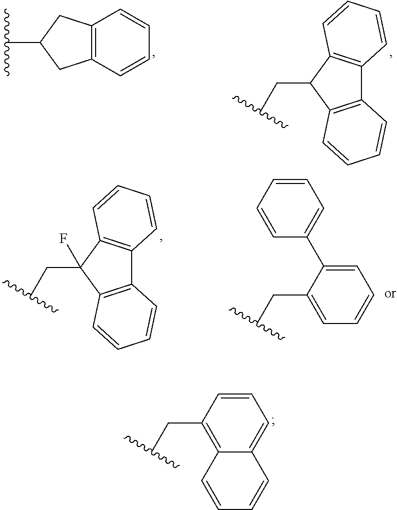
(d) R₁₆ is 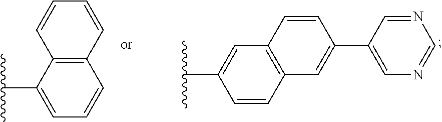 [SMILES: *c1cccc2ccccc12 ; *c1ccc2cc(-c3cncnc3)ccc2c1]
(e) R₁₆ is 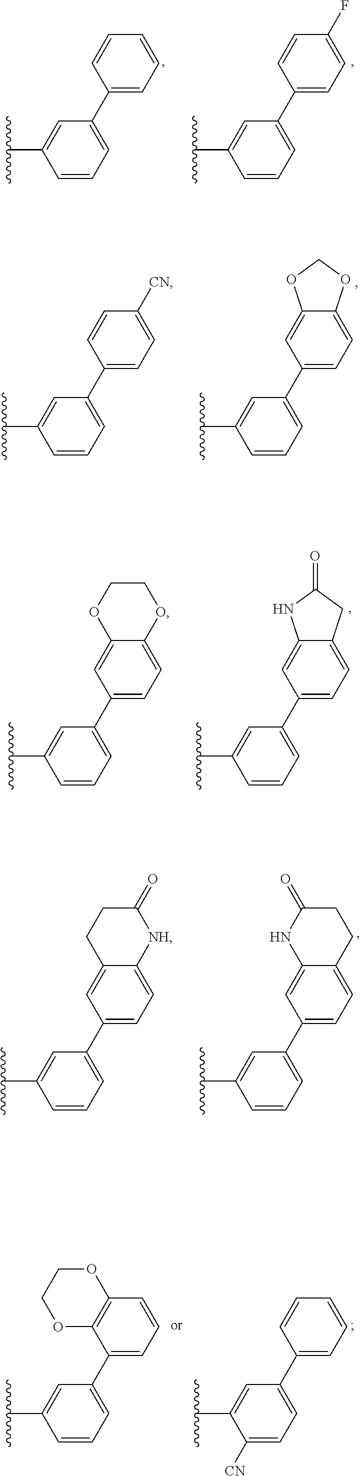
or
(f) R₁₆ is

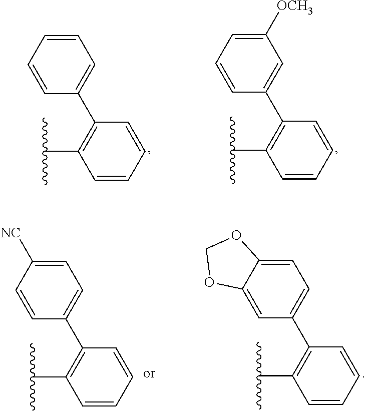 [SMILES: *c1ccccc1-c1ccccc1 ; *c1ccccc1-c1cccc(OC)c1 ; *c1ccccc1-c1ccc(C#N)cc1 ; *c1ccccc1-c1ccc2c(c1)OCO2]

37-42. (canceled)

43. The compound of claim 1 having the structure: 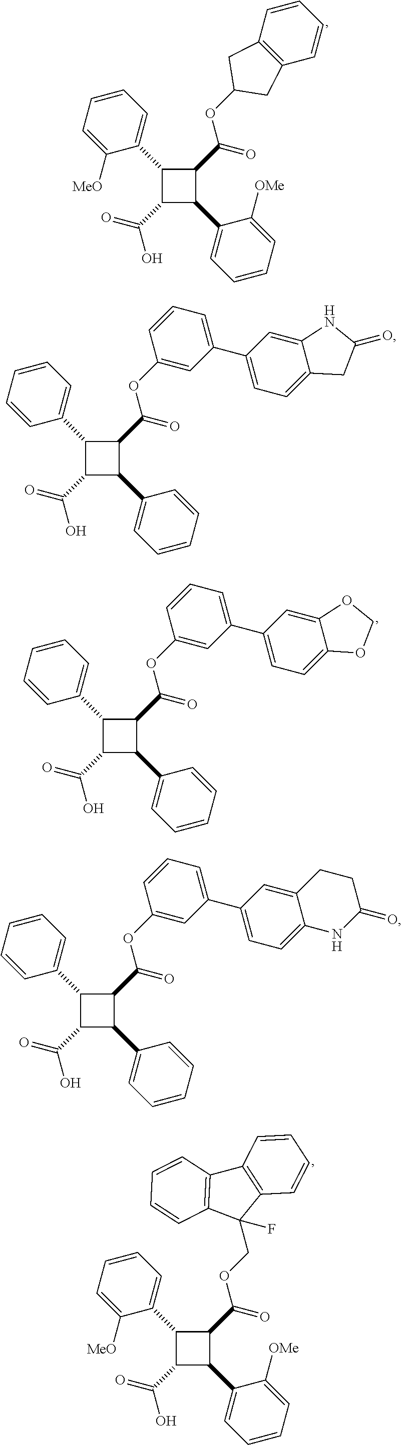 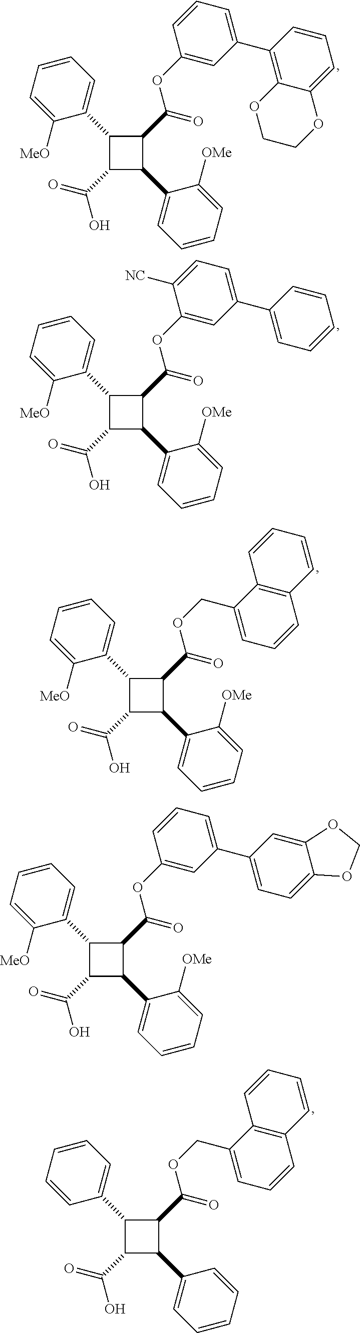 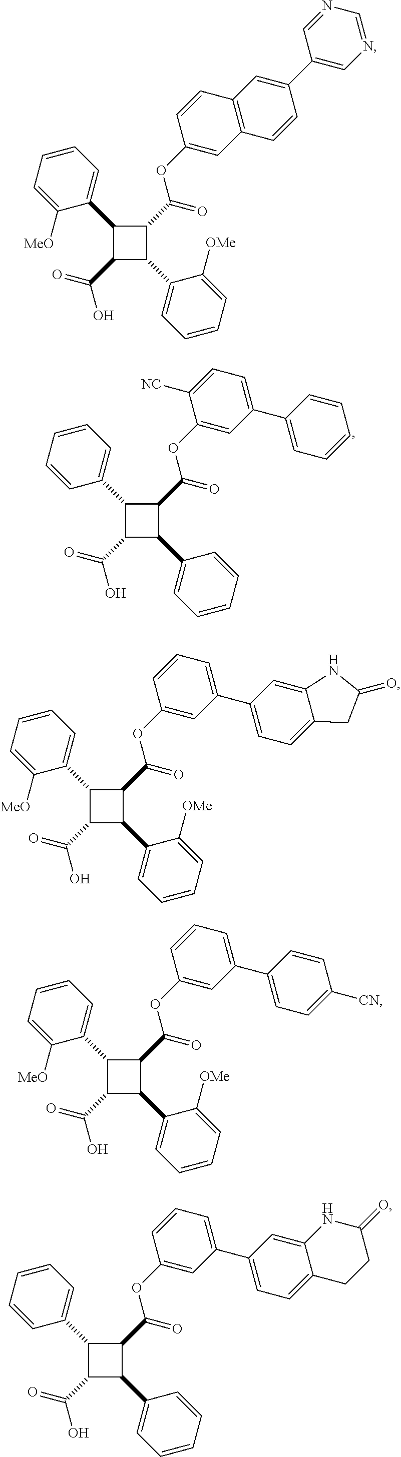 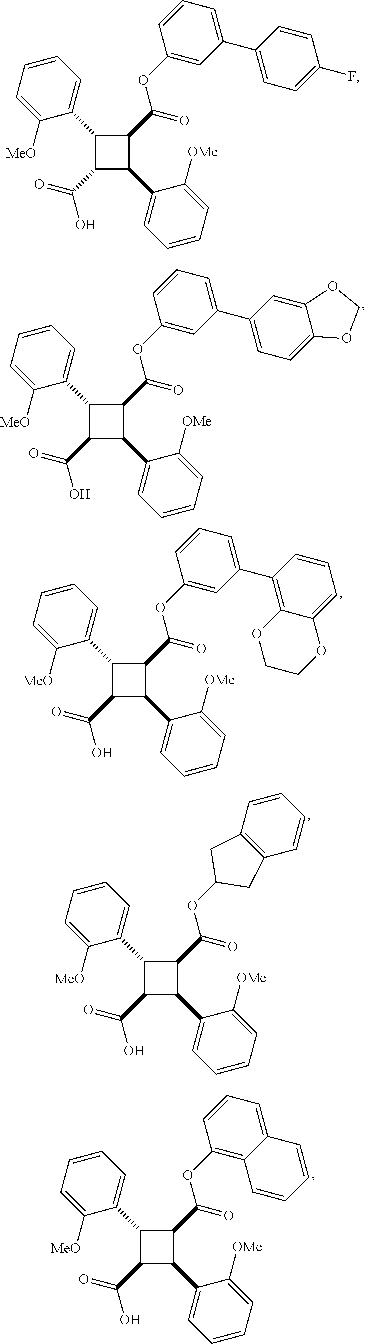 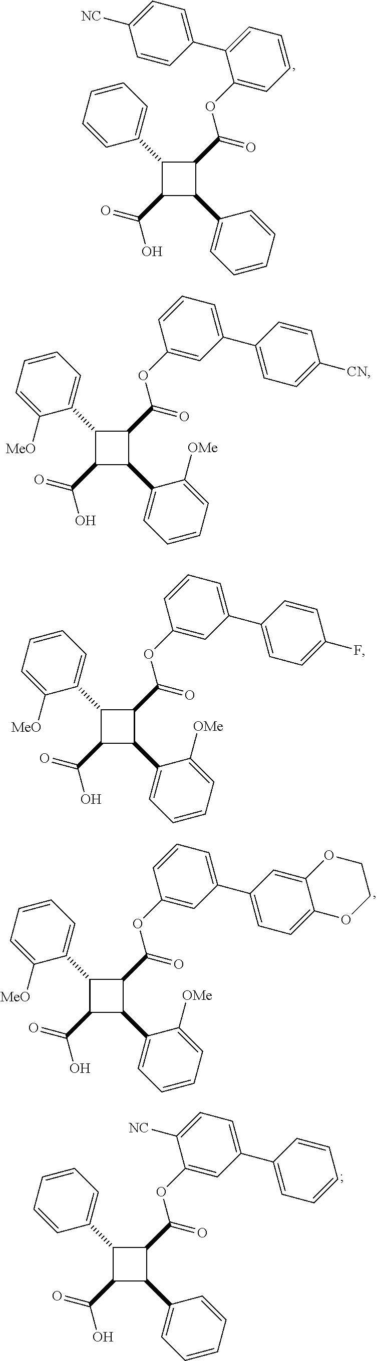 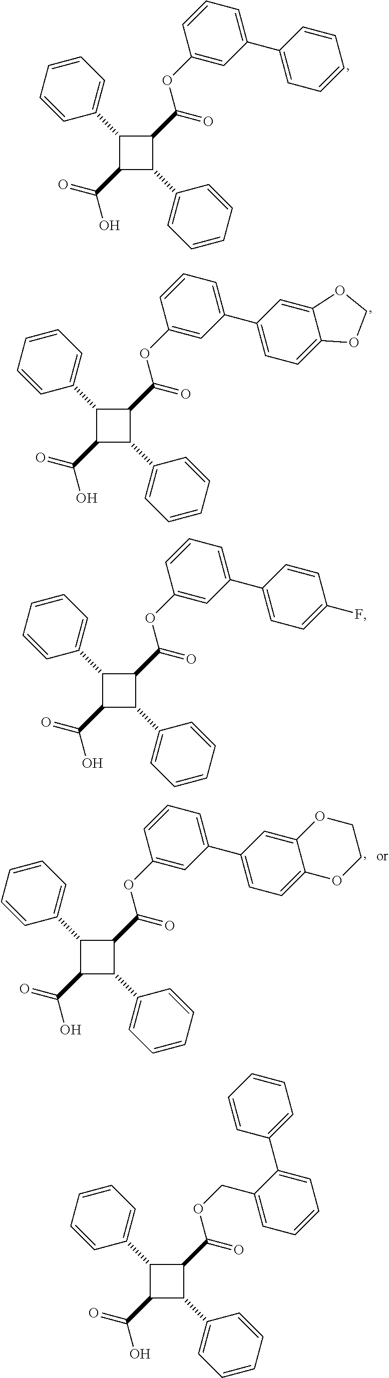
or an enantiomer or racemate thereof;
or a pharmaceutically acceptable salt thereof.

44-45. (canceled)

46. A pharmaceutical composition comprising the compound of claim 1 and a pharmaceutically acceptable carrier.

47. A method of inhibiting binding of a Fatty Acid Binding Protein (FABP) to a FABP ligand in a cell comprising contacting the FABP with the compound of claim 1; or a method of treating pain in a subject comprising administering to the subject the compound of claim 1.

48. The method of claim 47, wherein
(a) the FABP ligand is an endocannabinoid;
(b) the FABP ligand is anandamide (AEA) or 2-arachidonoylglycerol (2-AG);
(c) the FABP is FABP5 or FABP7;
(d) the pain is nociceptive pain, neurogenic pain, inflammatory pain, or chronic pain;
(e) the compound is administered in an effective amount to inhibit binding of FABP to a FABP ligand in the subject; or
(f) the compound is administered in an effective amount to inhibit binding of FABP to a FABP ligand in the subject.

49-54. (canceled)

55. A method of treating cancer in a subject comprising administering to the subject an effective amount of a compound having the structure: 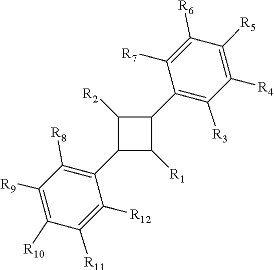
wherein
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I 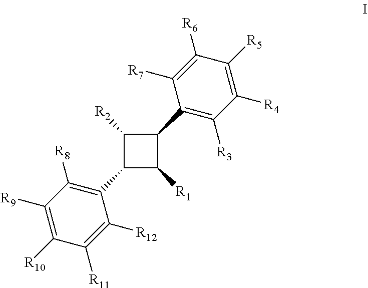
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl, or
wherein when the compound has the stereochemistry of structure I and when one of R₁ or R₂ is —C(═O)OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene or —C(═O)O-alkyl-R₁₄ where the alkyl is a C₁ alkyl and the R₁₄ is 9-fluorene;
wherein when the compound has the stereochemistry of structure II 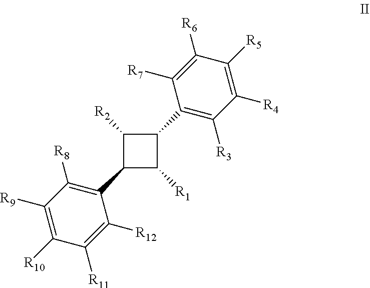
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III 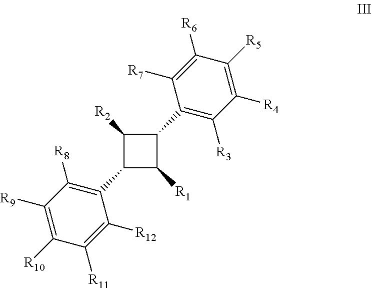
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

56. (canceled)

57. The method of claim 55, wherein
(a) the cancer is prostate cancer, skin cancer or breast cancer;
(b) the cancer is drug-resistant prostate cancer;
(c) the cancer is metastatic prostate cancer;
(d) the method further comprising administering a taxane in combination with the compound to the subject;
(e) the method further comprising administering a taxane in combination with the compound to the subject; wherein the taxane is docetaxel or cabazitaxel.

58-61. (canceled)

62. A method of treating pain in a subject without the side-effects of excessive inhibition of FABP3 comprising administering to the subject an effective amount of a compound having the structure: 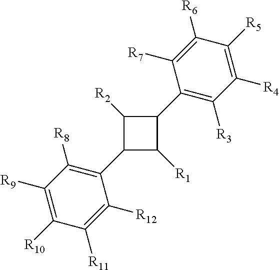
wherein
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I 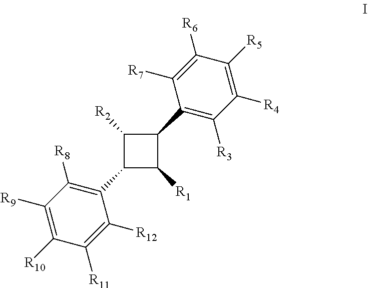
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure II 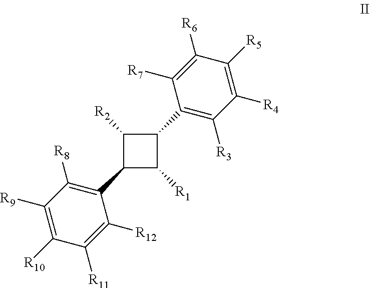
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure III 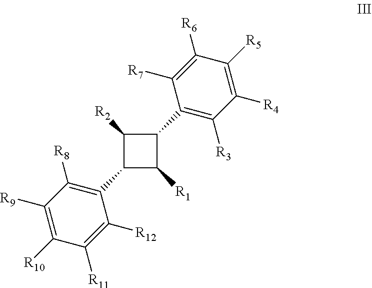
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
or a method comprising administering to the subject an effective amount of a compound having the structure: 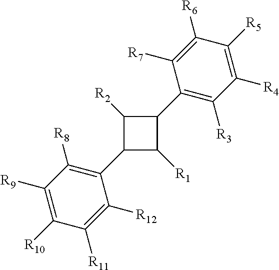
wherein
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when the compound has the stereochemistry of structure I 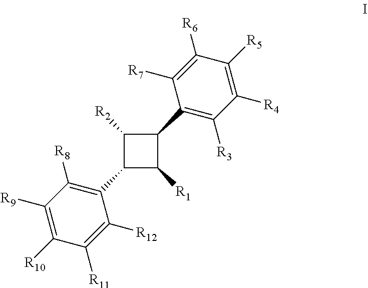
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene, 2-indane, 2-methylphenyl, 2-iodophenyl, 2-ethynylphenyl, 2-(1,1′-biphenyl), 3-(1,1′-biphenyl), 4-(1,1′-biphenyl), 2-(2′-hydroxy-1,1′-biphenyl), 2,4,5-trichlorophenyl, 2-phenylcyclohexyl, 1-naphthalene-6-acetamide, 1-naphthalene-5-ethyne, cyclohexyl, 3-[1-(3,6,9-trioxa-dodecanyl)-1,2,3-triazol-4-yl]phenyl, or —C(═O)O-alkyl-R₁₄ where the alkyl is a branched C₂ alkyl and the R₁₄ is phenyl or the alkyl is a C₁ alkyl and the R₁₄ is phenyl, 4-methoxyphenyl, 4-fluorophenyl, 4-bromophenyl, or 9-fluorene,
wherein when one of R₁ or R₂ is —C(═O)OH and R₄, R₅, R₆, R₇, R₉, R₁₀, R₁₁ and R₁₂ are each H and R₃ and R₈ are each —OCH₃, then the other of R₁ or R₂ is other than —C(═O)OR₁₃ where R₁₃ is 1-naphthalene, 2-naphthalene, 2-phenylcyclohexyl, or —C(═O)O-alkyl-R₁₄ where the alkyl is a C₁ alkyl and the R₁₄ is 9-fluorene,
wherein when one of R₁ or R₂ is —C(═O)OH and R₄, R₅, R₆, R₇, R₉, R₁₀, R₁₁ and R₁₂ are each H and R₃ and R₈ are each —Cl or —Br, then the other of R₁ or R₂ is other than —C(═O)OR₁₃ where R₁₃ is 2-phenylcyclohexyl,
wherein when one of R₁ or R₂ is —C(═O)OH and R₄, R₅, R₆, R₉, R₁₀, and R₁₁ are each H and R₃, R₇, Re and R₁₂ are each —Cl, then the other of R₁ or R₂ is other than —C(═O)OR₁₃ where R₁₃ is 2-phenylcyclohexyl,
wherein when one of R₁ or R₂ is —C(═O)OH and R₃, R₄, R₆, R₇, R₈, R₉, R₁₁, and R₁₂ are each H and R₅ and R₁₀ are each —OH, then the other of R₁ or R₂ is other than —C(═O)OR₁₃ where R₁₃ is 1-naphthalene,
wherein when one of R₁ or R₂ is —C(═O)OH and R₃, R₆, R₇, R₈, R₁₁, and R₁₂ are each H, R₄ and R₉ are each OCH₃, and R₅ and R₁₀ are each —OH, then the other of R₁ or R₂ is other than —C(═O)OR₁₃ where R₁₃ is 1-naphthalene,
wherein when the compound has the stereochemistry of structure II 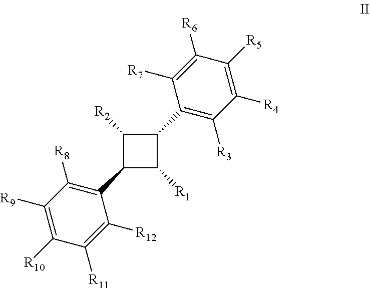
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
wherein when one of R₁ or R₂ is —C(═O)OH and R₃, R₄, R₅, R₆, R₇, R₈, R₉, R₁₀, R₁₁ and R₁₂ are each H, then the other of R₁ or R₂ is other than —C(═O)OR₁₃ where R₁₃ is methyl, 2-propyl, pentyl, octyl, —CH₂C(O)CH₃, 1-naphthalene, 2-naphthalene or 2-methylphenyl, or —C(═O)O-alkyl-R₁₄ where the alkyl is a branched C₂ alkyl and the R₁₄ is phenyl,
wherein when the compound has the stereochemistry of structure III 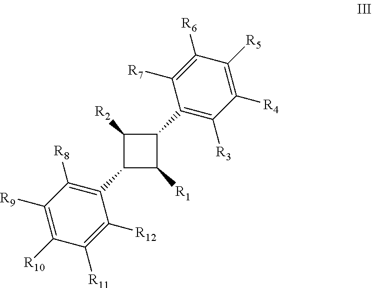
then
one of R₁ or R₂ is —C(═O)OH and the other of R₁ or R₂ is —C(═O)OR₁₃ or —C(═O)O-alkyl-R₁₄,
wherein
R₁₃ is cycloalkyl, aryl or heteroaryl, and
R₁₄ is cycloalkyl, aryl or heteroaryl; and
R₃, R₄, R₅, R₆, R₇, R₈, R₉, R₁₀, R₁₁ and R₁₂ are each independently, H, —OH, —OR₁₅, or halogen
wherein R₁₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

63. The method of claim 62, wherein the pain is nociceptive pain, neurogenic pain, inflammatory pain, or chronic pain.

64. The method of claim 62 comprising administering to the subject an effective amount of a compound having the structure: 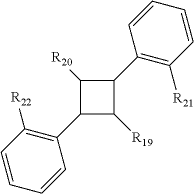
wherein
one of R₁₉ or R₂₀ is —C(═O)OH and the other of R₁₉ or R₂₀ is —C(═O)OR₂₃ or —C(═O)O-alkyl-R₂₄,
wherein
R₂₃ is cycloalkyl, aryl or heteroaryl, and
R₂₄ is cycloalkyl, aryl or heteroaryl; and
R₂₁ and R₂₂ are each independently, H, —OH, —OR₂₅, or halogen
wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure VII 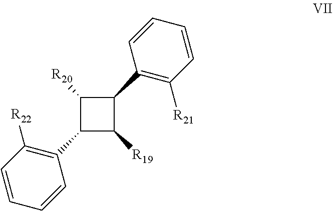
then
one of R₁₉ or R₂₀ is —C(═O)OH and the other of R₁₉ or R₂₀ is —C(═O)OR₂₃ or —C(═O)O-alkyl-R₂₄,
wherein
R₂₃ is cycloalkyl, aryl or heteroaryl, and
R₂₄ is cycloalkyl, aryl or heteroaryl; and
R₂₁ and R₂₂ are each independently, H, —OH, —OR₂₅, or halogen
wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure VIII 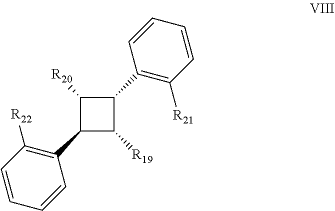
then
one of R₁₉ or R₂₀ is —C(═O)OH and the other of R₁₉ or R₂₀ is —C(═O)OR₂₃ or —C(═O)O-alkyl-R₂₄,
wherein
R₂₃ is cycloalkyl, aryl or heteroaryl, and
R₂₄ is cycloalkyl, aryl or heteroaryl; and
R₂₁ and R₂₂ are each independently, H, —OH, —OR₂₅, or halogen
wherein R₂₅ is H, C₁₋₁₀ alkyl, C₂₋₁₀ alkenyl, C₂₋₁₀ alkynyl, aryl, or heteroaryl,
wherein when the compound has the stereochemistry of structure IX 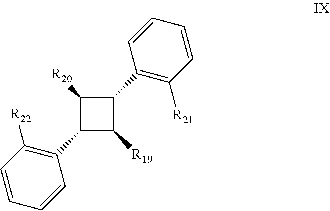
then
one of R₁₉ or R₂₀ is —C(═O)OH and the other of R₁₉ or R₂₀ is —C(═O)OR₂₃ or —C(═O)O-alkyl-R₂₄,
wherein
R₂₃ is cycloalkyl, aryl or heteroaryl, and
R₂₄ is cycloalkyl, aryl or heteroaryl; and
R₂₁ and R₂₂ are each independently, H, —OH, —OR₂₅, or halogen
wherein R₂₅ is H, C₁₋₁₀ alkyl, C₂₋₁₀ alkenyl, C₂₋₁₀ alkynyl, aryl, or heteroaryl,
or an enantiomer or racemate thereof;
or a pharmaceutically acceptable salt thereof.

65. (canceled)
